SEQUENCE LISTING (140 sequences)

SEQ ID NO: 1 - protein, 953 aa, Homo sapiens
IPVIEPSVPELVVKPGATVTLRCVGNGSVEWDGPPSPHWTLYSDGSSSILSTNNATFQNTGTYRCTEPGDPLGGSAAIHLYVKDPARPWNVLAQEVVVFEDQDALLPCLLTDPVLEAGVSLVRVRGRPLMRHTNYSFSPWHGFTIHRAKFIQSQDYQCSALMGGRKVMSISIRLKVQKVIPGPPALTLVPAELVRIRGEAAQIVCSASSVDVNFDVFLQHNNTKLAIPQQSDFHNNRYQKVLTLNLDQVDFQHAGNYSCVASNVQGKHSTSMFFRVVESAYLNLSSEQNLIQEVTVGEGLNLKVMVEAYPGLQGFNWTYLGPFSDHQPEPKLANATTKDTYRHTFTLSLPRLKPSEAGRYSFLARNPGGWRALTFELTLRYPPEVSVIWTFINGSGTLLCAASGYPQPNVTWLQCSGHTDRCDEAQVLQVWDDPYPEVLSQEPFHKVTVQSLLTVETLEHNQTYECRAHNSVGSGSWAFIPISAGAHTHPPDEFLFTPVVVACMSIMALLLLLLLLLLYKYKQKPKYQVRWKIIESYEGNSYTFIDPTQLPYNEKWEFPRNNLQFGKTLGAGAFGKVVEATAFGLGKEDAVLKVAVKMLKSTAHADEKEALMSELKIMSHLGQHENIVNLLGACTHGGPVLVITEYCCYGDLLNFLRRKAEAMLGPSLSPGQDPEGGVDYKNIHLEKKYVRRDSGFSSQGVDTYVEMRPVSTSSNDSFSEQDLDKEDGRPLELRDLLHFSSQVAQGMAFLASKNCIHRDVAARNVLLTNGHVAKIGDFGLARDIMNDSNYIVKGNARLPVKWMAPESIFDCVYTVQSDVWSYGILLWEIFSLGLNPYPGILVNSKFYKLVKDGYQMAQPAFAPKNIYSIMQACWALEPTHRPTFQQICSFLQEQAQEDRRERDYTNLPSSSRSGGSGSSSSELEEESSSEHLTCCEQGDIAQPLLQPNNYQFC

SEQ ID NO: 2 - protein, 972 aa, Homo sapiens
MGPGVLLLLLVATAWHGQGIPVIEPSVPELVVKPGATVTLRCVGNGSVEWDGPPSPHWTLYSDGSSSILSTNNATFQNTGTYRCTEPGDPLGGSAAIHLYVKDPARPWNVLAQEVVVFEDQDALLPCLLTDPVLEAGVSLVRVRGRPLMRHTNYSFSPWHGFTIHRAKFIQSQDYQCSALMGGRKVMSISIRLKVQKVIPGPPALTLVPAELVRIRGEAAQIVCSASSVDVNFDVFLQHNNTKLAIPQQSDFHNNRYQKVLTLNLDQVDFQHAGNYSCVASNVQGKHSTSMFFRVVESAYLNLSSEQNLIQEVTVGEGLNLKVMVEAYPGLQGFNWTYLGPFSDHQPEPKLANATTKDTYRHTFTLSLPRLKPSEAGRYSFLARNPGGWRALTFELTLRYPPEVSVIWTFINGSGTLLCAASGYPQPNVTWLQCSGHTDRCDEAQVLQVWDDPYPEVLSQEPFHKVTVQSLLTVETLEHNQTYECRAHNSVGSGSWAFIPISAGAHTHPPDEFLFTPVVVACMSIMALLLLLLLLLLYKYKQKPKYQVRWKIIESYEGNSYTFIDPTQLPYNEKWEFPRNNLQFGKTLGAGAFGKVVEATAFGLGKEDAVLKVAVKMLKSTAHADEKEALMSELKIMSHLGQHENIVNLLGACTHGGPVLVITEYCCYGDLLNFLRRKAEAMLGPSLSPGQDPEGGVDYKNIHLEKKYVRRDSGFSSQGVDTYVEMRPVSTSSNDSFSEQDLDKEDGRPLELRDLLHFSSQVAQGMAFLASKNCIHRDVAARNVLLTNGHVAKIGDFGLARDIMNDSNYIVKGNARLPVKWMAPESIFDCVYTVQSDVWSYGILLWEIFSLGLNPYPGILVNSKFYKLVKDGYQMAQPAFAPKNIYSIMQACWALEPTHRPTFQQICSFLQEQAQEDRRERDYTNLPSSSRSGGSGSSSSELEEESSSEHLTCCEQGDIAQPLLQPNNYQFC

SEQ ID NO: 3 - protein, 20 aa, Unknown
  source: /note=“Description of Unknown Light chain leader sequence”
METDTLLLWVLLLWVPGSTG

SEQ ID NO: 4 - protein, 19 aa, Unknown
  source: /note=“Description of Unknown Heavy chain leader sequence”
MAVLGLLLCLVTFPSCVLS

SEQ ID NO: 5 - protein, 487 aa, Homo sapiens
IPVIEPSVPELVVKPGATVTLRCVGNGSVEWDGPPSPHWTLYSDGSSSILSTNNATFQNTGTYRCTEPGDPLGGSAAIHLYVKDPARPWNVLAQEVVVFEDQDALLPCLLTDPVLEAGVSLVRVRGRPLMRHTNYSFSPWHGFTIHRAKFIQSQDYQCSALMGGRKVMSISIRLKVQKVIPGPPALTLVPAELVRIRGEAAQIVCSASSVDVNFDVFLQHNNTKLAIPQQSDFHNNRYQKVLTLNLDQVDFQHAGNYSCVASNVQGKHSTSMFFRVVESAYLNLSSEQNLIQEVTVGEGLNLKVMVEAYPGLQGFNWTYLGPFSDHQPEPKLANATTKDTYRHTFTLSLPRLKPSEAGRYSFLARNPGGWRALTFELTLRYPPEVSVIWTFINGSGTLLCAASGYPQPNVTWLQCSGHTDRCDEAQVLQVWDDPYPEVLSQEPFHKVTVQSLLTVETLEHNQTYECRAHNSVGSGSWAFIPISAGAH

SEQ ID NO: 6 - protein, 719 aa, Homo sapiens
IPVIEPSVPELVVKPGATVTLRCVGNGSVEWDGPPSPHWTLYSDGSSSILSTNNATFQNTGTYRCTEPGDPLGGSAAIHLYVKDPARPWNVLAQEVVVFEDQDALLPCLLTDPVLEAGVSLVRVRGRPLMRHTNYSFSPWHGFTIHRAKFIQSQDYQCSALMGGRKVMSISIRLKVQKVIPGPPALTLVPAELVRIRGEAAQIVCSASSVDVNFDVFLQHNNTKLAIPQQSDFHNNRYQKVLTLNLDQVDFQHAGNYSCVASNVQGKHSTSMFFRVVESAYLNLSSEQNLIQEVTVGEGLNLKVMVEAYPGLQGFNWTYLGPFSDHQPEPKLANATTKDTYRHTFTLSLPRLKPSEAGRYSFLARNPGGWRALTFELTLRYPPEVSVIWTFINGSGTLLCAASGYPQPNVTWLQCSGHTDRCDEAQVLQVWDDPYPEVLSQEPFHKVTVQSLLTVETLEHNQTYECRAHNSVGSGSWAFIPISAGAHEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 506 aa, Macaca fascicularis
MGPGVLLLLLVVTAWHGQGIPVIEPSGPELVVKPGETVTLRCVGNGSVEWDGPISPHWTLYSDGPSSVLTTTNATFQNTRTYRCTEPGDPLGGSAAIHLYVKDPARPWNVLAKEVVVFEDQDALLPCLLTDPVLEAGVSLVRLRGRPLLRHTNYSFSPWHGFTIHRAKFIQGQDYQCSALMGSRKVMSISIRLKVQKVIPGPPALTLVPAELVRIRGEAAQIVCSASNIDVDFDVFLQHNTTKLAIPQRSDFHDNRYQKVLTLSLGQVDFQHAGNYSCVASNVQGKHSTSMFFRVVESAYLDLSSEQNLIQEVTVGEGLNLKVMVEAYPGLQGFNWTYLGPFSDHQPEPKLANATTKDTYRHTFTLSLPRLKPSEAGRYSFLARNPGGWRALTFELTLRYPPEVSVIWTSINGSGTLLCAASGYPQPNVTWLQCAGHTDRCDEAQVLQVWVDPHPEVLSQEPFQKVTVQSLLTAETLEHNQTYECRAHNSVGSGSWAFIPISAGAR

SEQ ID NO: 8 - protein, 740 aa, Macaca fascicularis
MGPGVLLLLLVVTAWHGQGIPVIEPSGPELVVKPGETVTLRCVGNGSVEWDGPISPHWTLYSDGPSSVLTTTNATFQNTRTYRCTEPGDPLGGSAAIHLYVKDPARPWNVLAKEVVVFEDQDALLPCLLTDPVLEAGVSLVRLRGRPLLRHTNYSFSPWHGFTIHRAKFIQGQDYQCSALMGSRKVMSISIRLKVQKVIPGPPALTLVPAELVRIRGEAAQIVCSASNIDVDFDVFLQHNTTKLAIPQRSDFHDNRYQKVLTLSLGQVDFQHAGNYSCVASNVQGKHSTSMFFRVVESAYLDLSSEQNLIQEVTVGEGLNLKVMVEAYPGLQGFNWTYLGPFSDHQPEPKLANATTKDTYRHTFTLSLPRLKPSEAGRYSFLARNPGGWRALTFELTLRYPPEVSVIWTSINGSGTLLCAASGYPQPNVTWLQCAGHTDRCDEAQVLQVWVDPHPEVLSQEPFQKVTVQSLLTAETLEHNQTYECRAHNSVGSGSWAFIPISAGARGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 122 aa, Artificial Sequence
  source: /note=“Description of Artificial Sequence Synthetic polypeptide”
EVQLQQSGPELVRPGASVKMSCKASGYTFTDNYMIWVKQSHGKSLEWIGDINPYNGGTTFNQKFKGKATLTVEKSSSTAYMQLNSLTSEDSAVYYCARESPYFSNLYVMDYWGQGTSVTVSS

SEQ ID NO: 10 - protein, 111 aa, Artificial Sequence
  source: /note=“Description of Artificial Sequence Synthetic polypeptide”
NIVLTQSPASLAVSLGQRATISCKASQSVDYDGDNYMNWYQQKPGQPPKLLIYAASNLESGIPARFSGSGSGTDFTLNIHPVEEEDAATYYCHLSNEDLSTFGGGTKLEIK

SEQ ID NO: 11 - protein, 121 aa, Artificial Sequence
  source: /note=“Description of Artificial Sequence Synthetic polypeptide”
EIQLQQSGPELVKPGASVKMSCKASGYTFSDFNIHWVKQKPGQGLEWIGYINPYTDVTVYNEKFKGKATLTSDRSSSTAYMDLSSLTSEDSAVYYCASYFDGTFDYALDYWGQGTSITVSS

SEQ ID NO: 12 - protein, 111 aa, Artificial Sequence
  source: /note=“Description of Artificial Sequence Synthetic polypeptide”
DVVVTQTPASLAVSLGQRATISCRASESVDNYGLSFMNWFQQKPGQPPKLLIYTASNLESGIPARFSGGGSRTDFTLTIDPVEADDAATYFCQQSKELPWTFGGGTRLEIK

SEQ ID NO: 13 - protein, 122 aa, Artificial Sequence
  source: /note=“Description of Artificial Sequence Synthetic polypeptide”
EIQLQQSGPDLMKPGASVKMSCKASGYIFTDYNMHWVKQNQGKSLEWMGEINPNNGVVVYNQKFKGTTTLTVDKSSSTAYMDLHSLTSEDSAVYYCTRALYHSNFGWYFDSWGKGTTLTVSS

SEQ ID NO: 14 - protein, 111 aa, Artificial Sequence
  source: /note=“Description of Artificial Sequence Synthetic polypeptide”
DIVLTQSPASLAVSLGQRATISCKASQSVDYDGDSHMNWYQQKPGQPPKLLIYTASNLESGIPARFSGSGSGADFTLTIHPVEEEDAATYYCQQGNEDPWTFGGGTRLEIK

SEQ ID NO: 15 - protein, 10 aa, Artificial Sequence
  source: /note=“Description of Artificial Sequence Synthetic peptide”
GYTFTDNYMI

SEQ ID NO: 16 - protein, 17 aa, Artificial Sequence
  source: /note=“Description of Artificial Sequence Synthetic peptide”
DINPYNGGTTFNQKFKG

SEQ ID NO: 17 - protein, 13 aa, Artificial Sequence
  source: /note=“Description of Artificial Sequence Synthetic peptide”
ESPYFSNLYVMDY

SEQ ID NO: 18 - protein, 15 aa, Artificial Sequence
  source: /note=“Description of Artificial Sequence Synthetic peptide”
KASQSVDYDGDNYMN

SEQ ID NO: 19 - protein, 7 aa, Artificial Sequence
  source: /note=“Description of Artificial Sequence Synthetic peptide”
AASNLES

SEQ ID NO: 20 - protein, 9 aa, Artificial Sequence
  source: /note=“Description of Artificial Sequence Synthetic peptide”
HLSNEDLST

SEQ ID NO: 21 - protein, 10 aa, Artificial Sequence
  source: /note=“Description of Artificial Sequence Synthetic peptide”
GYTFSDFNIH

SEQ ID NO: 22 - protein, 17 aa, Artificial Sequence
  source: /note=“Description of Artificial Sequence Synthetic peptide”
YINPYTDVTVYNEKFKG

SEQ ID NO: 23 - protein, 12 aa, Artificial Sequence
  source: /note=“Description of Artificial Sequence Synthetic peptide”
YFDGTFDYALDY

SEQ ID NO: 24 - protein, 15 aa, Artificial Sequence
  source: /note=“Description of Artificial Sequence Synthetic peptide”
RASESVDNYGLSFMN

SEQ ID NO: 25 - protein, 7 aa, Artificial Sequence
  source: /note=“Description of Artificial Sequence Synthetic peptide”
TASNLES

SEQ ID NO: 26 - protein, 9 aa, Artificial Sequence
  source: /note=“Description of Artificial Sequence Synthetic peptide”
QQSKELPWT

SEQ ID NO: 27 - protein, 10 aa, Artificial Sequence
  source: /note=“Description of Artificial Sequence Synthetic peptide”
GYIFTDYNMH

SEQ ID NO: 28 - protein, 17 aa, Artificial Sequence
  source: /note=“Description of Artificial Sequence Synthetic peptide”
EINPNNGVVVYNQKFKG

SEQ ID NO: 29 - protein, 13 aa, Artificial Sequence
  source: /note=“Description of Artificial Sequence Synthetic peptide”
ALYHSNFGWYFDS

SEQ ID NO: 30 - protein, 15 aa, Artificial Sequence
  source: /note=“Description of Artificial Sequence Synthetic peptide”
KASQSVDYDGDSHMN

SEQ ID NO: 31 - protein, 7 aa, Artificial Sequence
  source: /note=“Description of Artificial Sequence Synthetic peptide”
TASNLES

SEQ ID NO: 32 - protein, 9 aa, Artificial Sequence
  source: /note=“Description of Artificial Sequence Synthetic peptide”
QQGNEDPWT

SEQ ID NO: 33 - protein, 449 aa, Artificial Sequence
  source: /note=“Description of Artificial Sequence Synthetic polypeptide”
EVQLQQSGPELVRPGASVKMSCKASGYTFTDNYMIWVKQSHGKSLEWIGDINPYNGGTTFNQKFKGKATLTVEKSSSTAYMQLNSLTSEDSAVYYCARESPYFSNLYVMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 34 - protein, 218 aa, Artificial Sequence
  source: /note=“Description of Artificial Sequence Synthetic polypeptide”
NIVLTQSPASLAVSLGQRATISCKASQSVDYDGDNYMNWYQQKPGQPPKLLIYAASNLESGIPARFSGSGSGTDFTLNIHPVEEEDAATYYCHLSNEDLSTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 448 aa, Artificial Sequence
  source: /note=“Description of Artificial Sequence Synthetic polypeptide”
EIQLQQSGPELVKPGASVKMSCKASGYTFSDFNIHWVKQKPGQGLEWIGYINPYTDVTVYNEKFKGKATLTSDRSSSTAYMDLSSLTSEDSAVYYCASYFDGTFDYALDYWGQGTSITVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 36 - protein, 218 aa, Artificial Sequence
  source: /note=“Description of Artificial Sequence Synthetic polypeptide”
DVVVTQTPASLAVSLGQRATISCRASESVDNYGLSFMNWFQQKPGQPPKLLIYTASNLESGIPARFSGGGSRTDFTLTIDPVEADDAATYFCQQSKELPWTFGGGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 449 aa, Artificial Sequence
  source: /note=“Description of Artificial Sequence Synthetic polypeptide”
EIQLQQSGPDLMKPGASVKMSCKASGYIFTDYNMHWVKQNQGKSLEWMGEINPNNGVVVYNQKFKGTTTLTVDKSSSTAYMDLHSLTSEDSAVYYCTRALYHSNFGWYFDSWGKGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 38 - protein, 218 aa, Artificial Sequence
  source: /note=“Description of Artificial Sequence Synthetic polypeptide”
DIVLTQSPASLAVSLGQRATISCKASQSVDYDGDSHMNWYQQKPGQPPKLLIYTASNLESGIPARFSGSGSGADFTLTIHPVEEEDAATYYCQQGNEDPWTFGGGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 122 aa, Artificial Sequence
  source: /note=“Description of Artificial Sequence Synthetic polypeptide”
QVQLVQSGAEVKKPGSSVKVSCKASGYTFTDNYMIWVRQAPGQGLEWMGDINPYNGGTTFNQKFKGRVTITADKSTSTAYMELSSLRSEDTAVYYCARESPYFSNLYVMDYWGQGTLVTVSS

SEQ ID NO: 40 - protein, 122 aa, Artificial Sequence
  source: /note=“Description of Artificial Sequence Synthetic polypeptide”
QVQLVQSGAEVKKPGSSVKVSCKASGYTFTDNYMIWVRQAPGQGLEWMGDINPYNGGTTFNQKFKGRVTITVDKSTSTAYMELSSLRSEDTAVYYCARESPYFSNLYVMDYWGQGTLVTVSS

SEQ ID NO: 41 - protein, 122 aa, Artificial Sequence
  source: /note=“Description of Artificial Sequence Synthetic polypeptide”
QVQLVQSGAEVKKPGSSVKVSCKASGYTFTDNYMIWVRQAPGQGLEWIGDINPYNGGTTFNQKFKGRATLTVDKSTSTAYMELSSLRSEDTAVYYCARESPYFSNLYVMDYWGQGTLVTVSS

SEQ ID NO: 42 - protein, 121 aa, Artificial Sequence
  source: /note=“Description of Artificial Sequence Synthetic polypeptide”
QVQLVQSGAEVKKPGSSVKVSCKASGYTFSDFNIHWVRQAPGQGLEWMGYINPYTDVTVYNEKFKGRVTITSDKSTSTAYMELSSLRSEDTAVYYCASYFDGTFDYALDYWGQGTLVTVSS

SEQ ID NO: 43 - protein, 121 aa, Artificial Sequence
  source: /note=“Description of Artificial Sequence Synthetic polypeptide”
QVQLVQSGAEVKKPGSSVKVSCKASGYTFSDFNIHWVRQAPGQGLEWIGYINPYTDVTVYNEKFKGRATLTSDKSTSTAYMELSSLRSEDTAVYYCASYFDGTFDYALDYWGQGTLVTVSS

SEQ ID NO: 44 - protein, 122 aa, Artificial Sequence
  source: /note=“Description of Artificial Sequence Synthetic polypeptide”
QVQLVQSGAEVKKPGSSVKVSCKASGYIFTDYNMHWVRQAPGQGLEWMGEINPNNGVVVYNQKFKGRVTITVDKSTSTAYMELSSLRSEDTAVYYCTRALYHSNFGWYFDSWGQGTLVTVSS

SEQ ID NO: 45 - protein, 122 aa, Artificial Sequence
  source: /note=“Description of Artificial Sequence Synthetic polypeptide”
QVQLVQSGAEVKKPGSSVKVSCKASGYIFTDYNMHWVRQAPGQGLEWMGEINPNNGVVVYNQKFKGTTTLTVDKSTSTAYMELSSLRSEDTAVYYCTRALYHSNFGWYFDSWGQGTLVTVSS

SEQ ID NO: 46 - protein, 111 aa, Artificial Sequence
  source: /note=“Description of Artificial Sequence Synthetic polypeptide”
EIVLTQSPATLSLSPGERATLSCKASQSVDYDGDNYMNWYQQKPGQAPRLLIYAASNLESGIPARFSGSGSGTDFTLTISSLEPEDFAVYYCHLSNEDLSTFGGGTKVEIK

SEQ ID NO: 47 - protein, 111 aa, Artificial Sequence
  source: /note=“Description of Artificial Sequence Synthetic polypeptide”
NIVLTQSPATLSLSPGERATLSCKASQSVDYDGDNYMNWYQQKPGQAPRLLIYAASNLESGIPARFSGSGSGTDFTLTISSLEPEDFAVYYCHLSNEDLSTFGGGTKVEIK

SEQ ID NO: 48 - protein, 111 aa, Artificial Sequence
  source: /note=“Description of Artificial Sequence Synthetic polypeptide”
EIVLTQSPATLSLSPGERATLSCRASESVDNYGLSFMNWYQQKPGQAPRLLIYTASNLESGIPARFSGSGSGTDFTLTISSLEPEDFAVYYCQQSKELPWTFGQGTKVEIK

SEQ ID NO: 49 - protein, 111 aa, Artificial Sequence
  source: /note=“Description of Artificial Sequence Synthetic polypeptide”
EIVLTQSPATLSLSPGERATLSCRASESVDNYGLSFMNWYQQKPGQAPRLLIYTASNLESGIPARFSGSGSRTDFTLTISSLEPEDFAVYYCQQSKELPWTFGQGTKVEIK

SEQ ID NO: 50 - protein, 111 aa, Artificial Sequence
  source: /note=“Description of Artificial Sequence Synthetic polypeptide”
EIVVTQSPATLSLSPGERATLSCRASESVDNYGLSFMNWFQQKPGQAPRLLIYTASNLESGIPARFSGSGSRTDFTLTISSLEPEDFAVYYCQQSKELPWTFGQGTKVEIK

SEQ ID NO: 51 - protein, 111 aa, Artificial Sequence
  source: /note=“Description of Artificial Sequence Synthetic polypeptide”
EIVLTQSPATLSLSPGERATLSCKASQSVDYDGDSHMNWYQQKPGQAPRLLIYTASNLESGIPARFSGSGSGTDFTLTISSLEPEDFAVYYCQQGNEDPWTFGQGTKVEIK

SEQ ID NO: 52 - protein, 111 aa, Artificial Sequence
  source: /note=“Description of Artificial Sequence Synthetic polypeptide”
DIVLTQSPATLSLSPGERATLSCKASQSVDYDGDSHMNWYQQKPGQAPRLLIYTASNLESGIPARFSGSGSGADFTLTISSLEPEDFAVYYCQQGNEDPWTFGQGTKVEIK

SEQ ID NO: 53 - protein, 449 aa, Artificial Sequence
  source: /note=“Description of Artificial Sequence Synthetic polypeptide”
QVQLVQSGAEVKKPGSSVKVSCKASGYTFTDNYMIWVRQAPGQGLEWMGDINPYNGGTTFNQKFKGRVTITADKSTSTAYMELSSLRSEDTAVYYCARESPYFSNLYVMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 54 - protein, 449 aa, Artificial Sequence
  source: /note=“Description of Artificial Sequence Synthetic polypeptide”
QVQLVQSGAEVKKPGSSVKVSCKASGYTFTDNYMIWVRQAPGQGLEWMGDINPYNGGTTFNQKFKGRVTITVDKSTSTAYMELSSLRSEDTAVYYCARESPYFSNLYVMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 55 - protein, 449 aa, Artificial Sequence
  source: /note=“Description of Artificial Sequence Synthetic polypeptide”
QVQLVQSGAEVKKPGSSVKVSCKASGYTFTDNYMIWVRQAPGQGLEWIGDINPYNGGTTFNQKFKGRATLTVDKSTSTAYMELSSLRSEDTAVYYCARESPYFSNLYVMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 56 - protein, 448 aa, Artificial Sequence
  source: /note=“Description of Artificial Sequence Synthetic polypeptide”
QVQLVQSGAEVKKPGSSVKVSCKASGYTFSDFNIHWVRQAPGQGLEWMGYINPYTDVTVYNEKFKGRVTITSDKSTSTAYMELSSLRSEDTAVYYCASYFDGTFDYALDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 57 - protein, 448 aa, Artificial Sequence
  source: /note=“Description of Artificial Sequence Synthetic polypeptide”
QVQLVQSGAEVKKPGSSVKVSCKASGYTFSDFNIHWVRQAPGQGLEWIGYINPYTDVTVYNEKFKGRATLTSDKSTSTAYMELSSLRSEDTAVYYCASYFDGTFDYALDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 58 - protein, 449 aa, Artificial Sequence
  source: /note=“Description of Artificial Sequence Synthetic polypeptide”
QVQLVQSGAEVKKPGSSVKVSCKASGYIFTDYNMHWVRQAPGQGLEWMGEINPNNGVVVYNQKFKGRVTITVDKSTSTAYMELSSLRSEDTAVYYCTRALYHSNFGWYFDSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 59 - protein, 449 aa, Artificial Sequence
  source: /note=“Description of Artificial Sequence Synthetic polypeptide”
QVQLVQSGAEVKKPGSSVKVSCKASGYIFTDYNMHWVRQAPGQGLEWMGEINPNNGVVVYNQKFKGTTTLTVDKSTSTAYMELSSLRSEDTAVYYCTRALYHSNFGWYFDSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 60 - protein, 218 aa, Artificial Sequence
  source: /note=“Description of Artificial Sequence Synthetic polypeptide”
EIVLTQSPATLSLSPGERATLSCKASQSVDYDGDNYMNWYQQKPGQAPRLLIYAASNLESGIPARFSGSGSGTDFTLTISSLEPEDFAVYYCHLSNEDLSTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 218 aa, Artificial Sequence
  source: /note=“Description of Artificial Sequence Synthetic polypeptide”
NIVLTQSPATLSLSPGERATLSCKASQSVDYDGDNYMNWYQQKPGQAPRLLIYAASNLESGIPARFSGSGSGTDFTLTISSLEPEDFAVYYCHLSNEDLSTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 218 aa, Artificial Sequence
  source: /note=“Description of Artificial Sequence Synthetic polypeptide”
EIVLTQSPATLSLSPGERATLSCRASESVDNYGLSFMNWYQQKPGQAPRLLIYTASNLESGIPARFSGSGSGTDFTLTISSLEPEDFAVYYCQQSKELPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 218 aa, Artificial Sequence
  source: /note=“Description of Artificial Sequence Synthetic polypeptide”
EIVLTQSPATLSLSPGERATLSCRASESVDNYGLSFMNWYQQKPGQAPRLLIYTASNLESGIPARFSGSGSRTDFTLTISSLEPEDFAVYYCQQSKELPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 218 aa, Artificial Sequence
  source: /note=“Description of Artificial Sequence Synthetic polypeptide”
EIVVTQSPATLSLSPGERATLSCRASESVDNYGLSFMNWFQQKPGQAPRLLIYTASNLESGIPARFSGSGSRTDFTLTISSLEPEDFAVYYCQQSKELPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - protein, 218 aa, Artificial Sequence
  source: /note=“Description of Artificial Sequence Synthetic polypeptide”
EIVLTQSPATLSLSPGERATLSCKASQSVDYDGDSHMNWYQQKPGQAPRLLIYTASNLESGIPARFSGSGSGTDFTLTISSLEPEDFAVYYCQQGNEDPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 218 aa, Artificial Sequence
  source: /note=“Description of Artificial Sequence Synthetic polypeptide”
DIVLTQSPATLSLSPGERATLSCKASQSVDYDGDSHMNWYQQKPGQAPRLLIYTASNLESGIPARFSGSGSGADFTLTISSLEPEDFAVYYCQQGNEDPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 158 aa, Homo sapiens
EEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQGHERQSEGS

SEQ ID NO: 68 - protein, 222 aa, Homo sapiens
NEPLEMWPLTQNEECTVTGFLRDKLQYRSRLQYMKHYFPINYKISVPYEGVFRIANVTRLQRAQVSERELRYLWVLVSLSATESVQDVLLEGHPSWKYLQEVQTLLLNVQQGLTDVEVSPKVESVLSLLNAPGPNLKLVRPKALLDNCFRVMELLYCSCCKQSSVLNWQDCEVPSPQSCSPEPSLQYAATQLYPPPPWSPSSPPHSTGSVRPVRAQGEGLLP

SEQ ID NO: 69 - protein, 25 aa, Homo sapiens [sequence omitted]

SEQ ID NO: 70 - protein, 14 aa, Homo sapiens
WVRQAPGQGLEWMG

SEQ ID NO: 71 - protein, 32 aa, Homo sapiens
RVTITADKSTSTAYMELSSLRSEDTAVYYCAR

SEQ ID NO: 72 - protein, 11 aa, Homo sapiens
WGQGTLVTVSS

SEQ ID NO: 73 - protein, 25 aa, Homo sapiens [sequence omitted]

SEQ ID NO: 74 - protein, 14 aa, Homo sapiens
WVRQAPGQGLEWMG

SEQ ID NO: 75 - protein, 32 aa, Homo sapiens; identical to SEQ ID NO: 71

SEQ ID NO: 76 - protein, 11 aa, Homo sapiens
WGQGTLVTVSS

SEQ ID NO: 77 - protein, 25 aa, Homo sapiens [sequence omitted]

SEQ ID NO: 78 - protein, 14 aa, Homo sapiens
WVRQAPGQGLEWMG

SEQ ID NO: 79 - protein, 32 aa, Homo sapiens; identical to SEQ ID NO: 71

SEQ ID NO: 80 - protein, 11 aa, Homo sapiens
WGQGTLVTVSS

SEQ ID NO: 81 - protein, 23 aa, Homo sapiens
EIVLTQSPATLSLSPGERATLSC

SEQ ID NO: 82 - protein, 15 aa, Homo sapiens
WYQQKPGQAPRLLIY

SEQ ID NO: 83 - protein, 32 aa, Homo sapiens
GIPARFSGSGSGTDFTLTISSLEPEDFAVYYC

SEQ ID NO: 84 - protein, 10 aa, Homo sapiens
FGGGTKVEIK

SEQ ID NO: 85 - protein, 23 aa, Homo sapiens
EIVLTQSPATLSLSPGERATLSC

SEQ ID NO: 86 - protein, 15 aa, Homo sapiens
WYQQKPGQAPRLLIY

SEQ ID NO: 87 - protein, 32 aa, Homo sapiens; identical to SEQ ID NO: 83

SEQ ID NO: 88 - protein, 10 aa, Homo sapiens
FGQGTKVEIK

SEQ ID NO: 89 - protein, 23 aa, Homo sapiens
EIVLTQSPATLSLSPGERATLSC

SEQ ID NO: 90 - protein, 15 aa, Homo sapiens
WYQQKPGQAPRLLIY

SEQ ID NO: 91 - protein, 32 aa, Homo sapiens; identical to SEQ ID NO: 83

SEQ ID NO: 92 - protein, 10 aa, Homo sapiens
FGQGTKVEIK

SEQ ID NO: 93 - protein, 719 aa, Mus sp.
APVIEPSGPELVVEPGETVTLRCVSNGSVEWDGPISPYWTLDPESPGSTLTTRNATFKNTGTYRCTELEDPMAGSTTIHLYVKDPAHSWNLLAQEVTVVEGQEAVLPCLITDPALKDSVSLMREGGRQVLRKTVYFFSPWRGFIIRKAKVLDSNTYVCKTMVNGRESTSTGIWLKVNRVHPEPPQIKLEPSKLVRIRGEAAQIVCSATNAEVGFNVILKRGDTKLEIPLNSDFQDNYYKKVRALSLNAVDFQDAGIYSCVASNDVGTRTATMNFQVVESAYLNLTSEQSLLQEVSVGDSLILTVHADAYPSIQHYNWTYLGPFFEDQRKLEFITQRAIYRYTFKLFLNRVKASEAGQYFLMAQNKAGWNNLTFELTLRYPPEVSVTWMPVNGSDVLFCDVSGYPQPSVTWMECRGHTDRCDEAQALQVWNDTHPEVLSQKPFDKVIIQSQLPIGTLKHNMTYFCKTHNSVGNSSQYFRAVSLGQSKQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 95 - protein, 107 aa, Homo sapiens [sequence omitted]

SEQ ID NO: 96 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 97 - protein, 268 aa, Homo sapiens
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 98 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 99 - protein, 272 aa, Homo sapiens
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 100 - protein, 113 aa, Artificial Sequence [sequence omitted]

SEQ ID NO: 101 - protein, 327 aa, Artificial Sequence; identical to SEQ ID NO: 94
  source: /note=“Description of Artificial Sequence Synthetic polypeptide”

SEQ ID NO: 102 - protein, 107 aa, Artificial Sequence [sequence omitted]

SEQ ID NO: 103 - protein, 107 aa, Artificial Sequence [sequence omitted]

SEQ ID NO: 104 - protein, 30 aa, Artificial Sequence
  source: /note=“Description of Artificial Sequence Synthetic polypeptide”
QVQLVESGGGVVQPGRSLRLDCKASGITFS

SEQ ID NO: 105 - protein, 5 aa, Artificial Sequence
  source: /note=“Description of Artificial Sequence Synthetic peptide”
NSGMH

SEQ ID NO: 106 - protein, 14 aa, Artificial Sequence
  source: /note=“Description of Artificial Sequence Synthetic peptide”
WVRQAPGKGLEWVA

SEQ ID NO: 107 - protein, 17 aa, Artificial Sequence
  source: /note=“Description of Artificial Sequence Synthetic peptide”
VIWYDGSKRYYADSVKG

SEQ ID NO: 108 - protein, 32 aa, Artificial Sequence
  source: /note=“Description of Artificial Sequence Synthetic polypeptide”
RFTISRDNSKNTLFLQMNSLRAEDTAVYYCAT

SEQ ID NO: 109 - protein, 4 aa, Artificial Sequence
  source: /note=“Description of Artificial Sequence Synthetic peptide”
NDDY

SEQ ID NO: 110 - protein, 11 aa, Artificial Sequence
  source: /note=“Description of Artificial Sequence Synthetic peptide”
WGQGTLVTVSS

SEQ ID NO: 111 - protein, 23 aa, Artificial Sequence
  source: /note=“Description of Artificial Sequence Synthetic peptide”
EIVLTQSPATLSLSPGERATLSC

SEQ ID NO: 112 - protein, 11 aa, Artificial Sequence
  source: /note=“Description of Artificial Sequence Synthetic peptide”
RASQSVSSYLA

SEQ ID NO: 113 - protein, 15 aa, Artificial Sequence
  source: /note=“Description of Artificial Sequence Synthetic peptide”
WYQQKPGQAPRLLIY

SEQ ID NO: 114 - protein, 7 aa, Artificial Sequence
  source: /note=“Description of Artificial Sequence Synthetic peptide”
DASNRAT

SEQ ID NO: 115 - protein, 32 aa, Artificial Sequence; identical to SEQ ID NO: 83
  source: /note=“Description of Artificial Sequence Synthetic polypeptide”

SEQ ID NO: 116 - protein, 9 aa, Artificial Sequence
  source: /note=“Description of Artificial Sequence Synthetic peptide”
QQSSNWPRT

SEQ ID NO: 117 - protein, 10 aa, Artificial Sequence
  source: /note=“Description of Artificial Sequence Synthetic peptide”
FGQGTKVEIK

SEQ ID NO: 118 - protein, 479 aa, Artificial Sequence
  source: /note=“Description of Artificial Sequence Synthetic polypeptide”
EVQLLESGGGEVQPGGSLRLSCAASGSVFSIDAMGWYRQAPGKQRELVAVLSGISSAKYAASAPGRFTISRDNAKNTVYLQMSSLRAEDTAVYYCYADVSTGWGRDAHGYWGQGTLVTVKPGGSGGSEVQLLESGGGEVQPGGSLRLSCAASGSVFSIDAMGWYRQAPGKQRELVAVLSGISSAKYAASAPGRFTISRDNAKNTVYLQMSSLRAEDTAVYYCYADVSTGWGRDAHGYWGQGTLVTVKPGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 119 - protein, 119 aa, Artificial Sequence
  source: /note=“Description of Artificial Sequence Synthetic polypeptide”
EVQLLESGGGEVQPGGSLRLSCAASGSVFSIDAMGWYRQAPGKQRELVAVLSGISSAKYAASAPGRFTISRDNAKNTVYLQMSSLRAEDTAVYYCYADVSTGWGRDAHGYWGQGTLVTV

SEQ ID NO: 120 - protein, 10 aa, Artificial Sequence
  source: /note=“Description of Artificial Sequence Synthetic peptide”
SGSVFSIDAM

SEQ ID NO: 121 - protein, 8 aa, Artificial Sequence
  source: /note=“Description of Artificial Sequence Synthetic peptide”
LSGISSAK

SEQ ID NO: 122 - protein, 16 aa, Artificial Sequence
  source: /note=“Description of Artificial Sequence Synthetic peptide”
YADVSTGWGRDAHGYW

SEQ ID NO: 123 - protein, 218 aa, Homo sapiens
PAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 124 - protein, 215 aa, Homo sapiens
PAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 125 - protein, 217 aa, Homo sapiens
PAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 126 - protein, 218 aa, Homo sapiens
PAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 127 - protein, 218 aa, Homo sapiens
PAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 128 - protein, 218 aa, Homo sapiens
PAPELLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 129 - protein, 14 aa, Homo sapiens
EPKSSDKTHTCPPC

SEQ ID NO: 130 - protein, 9 aa, Homo sapiens
DKTHTCPPC

SEQ ID NO: 131 - protein, 11 aa, Homo sapiens
ESKYGPPCPPC

SEQ ID NO: 132 - protein, 12 aa, Artificial Sequence
  source: /note=“Description of Artificial Sequence Synthetic peptide”
GQGTLVTVKPGG

SEQ ID NO: 133 - protein, 12 aa, Artificial Sequence
  source: /note=“Description of Artificial Sequence Synthetic peptide”
GQGTLVTVEPGG

SEQ ID NO: 134 - protein, 6 aa, Artificial Sequence
  source: /note=“Description of Artificial Sequence Synthetic peptide”
GGSGGS

SEQ ID NO: 135 - protein, 9 aa, Artificial Sequence
  source: /note=“Description of Artificial Sequence Synthetic peptide”
GGSGGSGGS

SEQ ID NO: 136 - protein, 12 aa, Artificial Sequence
  source: /note=“Description of Artificial Sequence Synthetic peptide”
GGSGGSGGSGGS

SEQ ID NO: 137 - protein, 15 aa, Artificial Sequence
  source: /note=“Description of Artificial Sequence Synthetic peptide”
GGSGGSGGSGGSGGS

SEQ ID NO: 138 - protein, 4 aa, Artificial Sequence
  source: /note=“Description of Artificial Sequence Synthetic peptide”
GGGG

SEQ ID NO: 139 - protein, 5 aa, Artificial Sequence
  source: /note=“Description of Artificial Sequence Synthetic peptide”
GGGGG

SEQ ID NO: 140 - protein, 6 aa, Artificial Sequence
  source: /note=“Description of Artificial Sequence Synthetic peptide”
GGGGGG

(19) United States
(12) Patent Application Publication
(10) Pub. No.: US 2020/0031944 A1
(43) Pub. Date: Jan. 30, 2020

(54) COMBINATION THERAPY FOR CANCER USING ANTI-GITR ANTIBODIES

(71) Applicant: Five Prime Therapeutics, Inc., South San Francisco, CA (US)

(72) Inventors: Susannah D. Barbee, San Francisco, CA (US); David Bellovin, San Jose, CA (US); Luis Borges, Houston, TX (US)

(73) Assignee: Five Prime Therapeutics, Inc., South San Francisco, CA (US)

(21) Appl. No.: 16/497,906

(22) PCT Filed: Mar. 29, 2018
(86) PCT No.: PCT/US2018/025034

§ 371 Date: Sep. 26, 2019

Related U.S. Application Data
(60) Provisional application No. 62/479,569, filed on Mar. 31, 2017.
(60) Provisional application No. 62/615,231, filed on Jan. 9, 2018.

(51) Int. Cl. C07K 16/28; A61P 35/00; A61K 39/395; A61K 31/7068; A61K 31/337; A61K 47/64; A61K 31/525; A61K 31/513; A61K 31/4745; A61K 31/282

(52) U.S. Cl. CPC C07K 16/2866; A61P 35/00; C07K 16/2878; A61K 39/3955; C07K 16/2818; A61K 31/7068; A61K 2039/505; A61K 47/643; A61K 31/525; A61K 31/513; A61K 31/4745; A61K 31/282; A61K 31/337

(57) ABSTRACT

Provided herein are methods of treating cancer with a combination of an anti glucocorticoid-induced tumor necrosis factor receptor (TNFR)-related protein (GITR)-antibody and an antibody that binds colony stimulating factor 1 receptor (CSF1R) or with a combination of an anti-GITR antibody and an antibody that binds programmed cell death protein 1 (PD-1).

### TECHNICAL FIELD

This application relates in some embodiments to methods of treating cancer with polypeptides that bind glucocorticoid-induced tumor necrosis factor receptor (TNFR)-related protein (GITR) in combination with antibodies that bind colony stimulating factor 1 receptor (CSF1R), or in combination with antibodies that bind programmed cell death protein 1 (PD-1).

### BACKGROUND

The glucocorticoid-induced tumor necrosis factor receptor (TNFR)-related protein (GITR) (also known as TNFRSF18, CD357, or AITR) is a member of the tumor necrosis factor receptor (TNFR) super family of proteins. Binding of GITR to GITR Ligand (GITRL, also known as TNFSF18) induces receptor trimerization and activation of downstream signaling pathways, including pathways characterized by NF-κB activation. GITR is highly expressed on the surface of certain regulatory T cells, but is expressed at low levels on conventional T cell subsets. Activation of T cells by certain stimuli leads to increased expression of GITR on regulatory T cells and on certain populations of effector T cells. GITR provides costimulatory signals to conventional T cells to enhance T cell responses to antigens. GITR is also believed to modulate suppression by regulatory T cells. For instance, GITR activation may reduce Treg lineage stability, may directly inhibit Treg suppressive activity, or may decrease the sensitivity of effector T cells to Treg-mediated suppression. See, e.g., S. Ronchetti et al., J. Immunol. Res., pp. 1-17 (2015); D. A. Knee et al., Eur. J. Cancer, 67: 1-10 (2016) for reviews related to GITR function.

Colony stimulating factor 1 receptor (referred to herein as CSF1R; also referred to in the art as FMS, FIM2, C-FMS, M-CSF receptor, and CD115) is a single-pass transmembrane receptor with an N-terminal extracellular domain (ECD) and a C-terminal intracellular domain with tyrosine kinase activity. Ligand binding of CSF1 or the interleukin 34 ligand (referred to herein as IL-34; Lin et al., Science 320: 807-11 (2008)) to CSF1R leads to receptor dimerization, upregulation of CSF1R protein tyrosine kinase activity, phosphorylation of CSF1R tyrosine residues, and downstream signaling events. CSF1R activation by CSF1 or IL-34 leads to the trafficking, survival, proliferation, and differentiation of monocytes and macrophages, as well as other monocytic cell lineages such as osteoclasts, dendritic cells, and microglia.

Many tumor cells or tumor stromal cells have been found to produce CSF1, which activates monocyte/macrophage cells through CSF1R. The level of CSF1 in tumors has been shown to correlate with the level of tumor-associated macrophages (TAMs) in the tumor. Higher levels of TAMs have been found to correlate with poorer patient prognoses in the majority of cancers. In addition, CSF1 has been found to promote tumor growth and progression to metastasis in, for example, human breast cancer xenografts in mice. See, e.g., Paulus et al., Cancer Res. 66: 4349-56 (2006). Further, CSF1R plays a role in osteolytic bone destruction in bone metastasis. See, e.g., Ohno et al., Mol. Cancer Ther. 5: 2634-43 (2006). TAMs promote tumor growth, in part, by suppressing anti-tumor T cell effector function through the release of immunosuppressive cytokines and the expression of T cell inhibitory surface proteins.

Genetic alterations in cancer provide a diverse set of antigens that can mediate anti-tumor immunity. Antigen recognition through T-cell receptors (TCRs) initiates T-cell-responses, which are regulated by a balance between activating and inhibitory signals. The inhibitory signals, or “immune checkpoints,” play an important role in normal tissues by preventing autoimmunity. Up-regulation of immune checkpoint proteins allows cancers to evade anti-tumor immunity. A particular immune checkpoint protein that has been the focus of clinical cancer immunotherapeutics is programmed cell death protein 1 (PD-1). Anti-PD-1 antibodies for use as monotherapies are currently being studied in clinical trials as a treatment for many different types of cancer and have been approved in a combination with an antibody against another immune checkpoint protein CTLA-4 for the treatment of metastatic melanoma, for example. The present invention relates to combinations of particular anti-GITR polypeptides with particular anti-PD-1 antibodies or with particular anti-CSF1R antibodies in cancer treatment.

### SUMMARY

The present disclosure includes, for example, methods of treating cancer in a subject comprising administering to the subject an anti-Colony Stimulating Factor 1 Receptor (CSF1R) antibody and an anti-Glucocorticoid-Induced TNFR-Related protein (GITR) antibody. For example, the disclosure includes methods of treating cancer in a subject comprising administering to the subject an anti-CSF1R antibody and an anti-GITR antibody, wherein the anti-GITR antibody is selected from: a) an antibody comprising a GITR binding domain (GITR-BD) comprising a CDR1 comprising the sequence of SEQ ID NO: 120, a CDR2 comprising the sequence of SEQ ID NO: 121, and a CDR3 comprising the sequence of SEQ ID NO: 122; b) an antibody comprising a GITR-BD comprising the sequence of SEQ ID NO: 119; c) a tetravalent molecule comprising two copies of a polypeptide having the structure (GITR-BD)-Linker-(GITR-BD)-Linker-Hinge-Fc, wherein (i) the GITR-BD comprises a CDR1 comprising the sequence of SEQ ID NO: 120, a CDR2 comprising the sequence of SEQ ID NO: 121, and a CDR3 comprising the sequence of SEQ ID NO: 122, (ii) the Linker is a polypeptide, (iii) the Hinge is a polypeptide derived from an immunoglobulin hinge region, and (iv) the Fc is an immunoglobulin Fc polypeptide; d) a tetravalent molecule comprising two copies of a polypeptide having the structure (GITR-BD)-Linker-(GITR-BD)-Linker-Hinge-Fc, wherein (i) the GITR-BD comprises the amino acid sequence of SEQ ID NO:119, (ii) the Linker is a polypeptide, (iii) the Hinge is a polypeptide derived from an immunoglobulin hinge region, and (iv) the Fc is an immunoglobulin Fc polypeptide; and e) a tetravalent molecule comprising two copies of a polypeptide comprising the sequence of SEQ ID NO: 118. In some embodiments, the anti-GITR antibody is a tetravalent molecule having the structure (GITR-BD)-Linker-(GITR-BD)-Linker-Hinge-Fc, wherein (i) the GITR-BD comprises (a) a CDR1 comprising the sequence of SEQ ID NO: 120, a CDR2 comprising the sequence of SEQ ID NO: 121, and a CDR3 comprising the sequence of SEQ ID NO: 122 or (b) the sequence of SEQ ID NO: 119, (ii) the Linker is a polypeptide comprising a sequence selected from SEQ ID NOs: 134-140, (iii) the Hinge is a polypeptide comprising a sequence selected from SEQ ID NOs: 129-133, and (iv) the Fc is an immunoglobulin Fc polypeptide comprising a sequence selected from SEQ ID NOs: 123-128.

In some embodiments, the anti-CSF1R antibody is selected from: a) an antibody comprising a heavy chain comprising the sequence of SEQ ID NO: 39 and a light chain comprising the sequence of SEQ ID NO: 46; b) an antibody comprising a heavy chain comprising a heavy chain (HC) complementarity determining region 1 (CDR1) comprising the sequence of SEQ ID NO: 15, an HC CDR2 comprising the sequence of SEQ ID NO: 16, and an HC CDR3 comprising the sequence of SEQ ID NO: 17, and a light chain comprising a light chain (LC) CDR1 comprising the sequence of SEQ ID NO: 18, a LC CDR2 comprising the sequence of SEQ ID NO: 19, and a LC CDR3 comprising the sequence of SEQ ID NO: 20; and c) an antibody comprising a heavy chain comprising the sequence of SEQ ID NO: 53 and a light chain comprising the sequence of SEQ ID NO: 60. In some embodiments, the anti-CSF1R antibody is a humanized antibody or is selected from: a Fab, an Fv, an scFv, a Fab′, and a (Fab′)₂. In some embodiments, the anti-CSF1R antibody blocks binding of both CSF1 and IL-34 to CSF1R. In some embodiments, the anti-CSF1R antibody inhibits ligand-induced CSF1R phosphorylation in vitro.

In some embodiments, the anti-CSF1R antibody and the anti-GITR antibody are administered concurrently or sequentially. In some embodiments, the anti-CSF1R antibody and the anti-GITR antibody are administered once every week, once every 2 weeks, once every 3 weeks, once every 4 weeks, or once every 5 weeks. In some embodiments, the anti-CSF1R antibody is administered at a dose of 0.1, 0.3, 0.5, 1, 2, 3, 4, 5, or 10 mg/kg. In some such embodiments, the anti-CSF1R antibody is administered at a dose of 1, 2, 3, or 4 mg/kg every 2 weeks or every 3 weeks.

In some embodiments, the cancer is selected from non-small cell lung cancer, melanoma, squamous cell carcinoma of the head and neck, ovarian cancer, pancreatic cancer, renal cell carcinoma, hepatocellular carcinoma, bladder cancer, malignant glioma, colorectal cancer, and endometrial cancer. In some embodiments, the cancer is recurrent or progressive after a therapy selected from one or more of surgery, chemotherapy, and radiation therapy. In some embodiments, administration of the anti-CSF1R antibody and the anti-GITR antibody results in a synergistic effect. In some embodiments, administration of the anti-CSF1R antibody and the anti-GITR antibody results in a synergistic inhibition of tumor growth in a mouse xenograft or syngeneic cancer model. In some embodiments, the method further comprises administering at least one chemotherapeutic agent.

The present disclosure also includes methods of treating cancer in a subject comprising administering to the subject an anti-Programmed cell Death 1 (PD-1) antibody and an anti-Glucocorticoid-Induced TNFR-Related protein (GITR) antibody, wherein the anti-GITR antibody is selected from: a) an antibody comprising a GITR binding domain (GITR-BD) comprising a CDR1 comprising the sequence of SEQ ID NO: 120, a CDR2 comprising the sequence of SEQ ID NO: 121, and a CDR3 comprising the sequence of SEQ ID NO: 122; b) an antibody comprising a GITR-BD comprising the sequence of SEQ ID NO: 119; c) a tetravalent molecule comprising two copies of a polypeptide having the structure (GITR-BD)-Linker-(GITR-BD)-Linker-Hinge-Fc, wherein (i) the GITR-BD comprises a CDR1 comprising the sequence of SEQ ID NO: 120, a CDR2 comprising the sequence of SEQ ID NO: 121, and a CDR3 comprising the sequence of SEQ ID NO: 122, (ii) the Linker is a polypeptide, (iii) the Hinge is a polypeptide derived from an immunoglobulin hinge region, and (iv) the Fc is an immunoglobulin Fc polypeptide; d) a tetravalent molecule comprising two copies of a polypeptide having the structure (GITR-BD)-Linker-(GITR-BD)-Linker-Hinge-Fc, wherein (i) the GITR-BD comprises the amino acid sequence of SEQ ID NO:119, (ii) the Linker is a polypeptide, (iii) the Hinge is a polypeptide derived from an immunoglobulin hinge region, and (iv) the Fc is an immunoglobulin Fc polypeptide; and e) a tetravalent molecule comprising two copies of a polypeptide comprising the sequence of SEQ ID NO: 118. In some embodiments, the anti-GITR antibody is a tetravalent molecule having the structure (GITR-BD)-Linker-(GITR-BD)-Linker-Hinge-Fc, wherein (i) the GITR-BD comprises (a) a CDR1 comprising the sequence of SEQ ID NO: 120, a CDR2 comprising the sequence of SEQ ID NO: 121, and a CDR3 comprising the sequence of SEQ ID NO: 122 or (b) the sequence of SEQ ID NO: 119, (ii) the Linker is a polypeptide comprising a sequence selected from SEQ ID NOs: 134-140, (iii) the Hinge is a polypeptide comprising a sequence selected from SEQ ID NOs: 129-133, and (iv) the Fc is an immunoglobulin Fc polypeptide comprising a sequence selected from SEQ ID NOs: 123-128.

In some embodiments, the anti-PD-1 antibody is selected from: a) an antibody comprising a heavy chain comprising the sequence of SEQ ID NO: 100 and a light chain comprising the sequence of SEQ ID NO: 102; b) an antibody comprising a heavy chain comprising a heavy chain (HC) complementarity determining region 1 (CDR1) having the sequence of SEQ ID NO: 105, an HC CDR2 having the sequence of SEQ ID NO: 107, and an HC CDR3 having the sequence of SEQ ID NO: 109, and a light chain comprising a light chain (LC) CDR1 having the sequence of SEQ ID NO: 112, a LC CDR2 having the sequence of SEQ ID NO: 114, and a LC CDR3 having the sequence of SEQ ID NO: 116; and c) an antibody comprising a heavy chain comprising the sequences of SEQ ID NOs: 100 and 101 and a light chain comprising the sequences of SEQ ID NOs: 102 and 103. In some embodiments, the anti-PD-1 antibody is a humanized antibody or is selected from a Fab, an Fv, an scFv, a Fab′, and a (Fab′)₂. In some embodiments, the anti-PD-1 antibody is nivolumab.

In some embodiments, the anti-PD-1 antibody and the anti-GITR antibody are administered concurrently or sequentially. In some embodiments, wherein the anti-PD-1 antibody and the anti-GITR antibody are administered once every week, once every 2 weeks, once every 3 weeks, once every 4 weeks, or once every 5 weeks. In some embodiments, the anti-PD-1 antibody is administered at a dose of 0.5, 1, 2, 3, 4, 5, or 10 mg/kg. In some such embodiments, the anti-PD-1 antibody is nivolumab and wherein the nivolumab is administered at a dose of 3 mg/kg every 2 weeks or at a flat dose of 240 mg every 2 weeks.

In some embodiments, the cancer is selected from non-small cell lung cancer, melanoma, squamous cell carcinoma of the head and neck, ovarian cancer, pancreatic cancer, renal cell carcinoma, hepatocellular carcinoma, bladder cancer, malignant glioma, colorectal cancer, and endometrial cancer. In some embodiments, the cancer is recurrent or progressive after a therapy selected from one or more of surgery, chemotherapy, and radiation therapy. In some embodiments, administration of the anti-PD-1 antibody and the anti-GITR antibody results in a synergistic effect. In some embodiments, administration of the anti-PD-1 antibody and the anti-GITR antibody results in a synergistic inhibition of tumor growth in a mouse xenograft or syngeneic cancer model.

In some embodiments, the subject has previously received PD-1/PD-L1 inhibitor therapy. In some embodiments, the subject is a PD-1/PD-L1 inhibitor inadequate responder or is refractory to a PD-1/PD-L1 inhibitor after at least 2 doses. In some embodiments, the method further comprises administering at least one chemotherapeutic agent.

The disclosure further encompasses compositions comprising an anti-GITR antibody for use in method of treating cancer, such as those described above, wherein the anti-GITR antibody is selected from: a) an antibody comprising a GITR binding domain (GITR-BD) comprising a CDR1 comprising the sequence of SEQ ID NO: 120, a CDR2 comprising the sequence of SEQ ID NO: 121, and a CDR3 comprising the sequence of SEQ ID NO: 122; b) an antibody comprising a GITR-BD comprising the sequence of SEQ ID NO: 119; c) a tetravalent molecule comprising two copies of a polypeptide having the structure (GITR-BD)-Linker-(GITR-BD)-Linker-Hinge-Fc, wherein (i) the GITR-BD comprises a CDR1 comprising the sequence of SEQ ID NO: 120, a CDR2 comprising the sequence of SEQ ID NO: 121, and a CDR3 comprising the sequence of SEQ ID NO: 122, (ii) the Linker is a polypeptide, (iii) the Hinge is a polypeptide derived from an immunoglobulin hinge region, and (iv) the Fc is an immunoglobulin Fc polypeptide; d) a tetravalent molecule comprising two copies of a polypeptide having the structure (GITR-BD)-Linker-(GITR-BD)-Linker-Hinge-Fc, wherein (i) the GITR-BD comprises the amino acid sequence of SEQ ID NO:119, (ii) the Linker is a polypeptide, (iii) the Hinge is a polypeptide derived from an immunoglobulin hinge region, and (iv) the Fc is an immunoglobulin Fc polypeptide; and e) a tetravalent molecule comprising two copies of a polypeptide comprising the sequence of SEQ ID NO: 118. In some embodiments of the compositions, the anti-CSF1R antibody is selected from: a) an antibody comprising a heavy chain comprising the sequence of SEQ ID NO: 39 and a light chain comprising the sequence of SEQ ID NO: 46; b) an antibody comprising a heavy chain comprising a heavy chain (HC) complementarity determining region 1 (CDR1) comprising the sequence of SEQ ID NO: 15, an HC CDR2 comprising the sequence of SEQ ID NO: 16, and an HC CDR3 comprising the sequence of SEQ ID NO: 17, and a light chain comprising a light chain (LC) CDR1 comprising the sequence of SEQ ID NO: 18, a LC CDR2 comprising the sequence of SEQ ID NO: 19, and a LC CDR3 comprising the sequence of SEQ ID NO: 20; and c) an antibody comprising a heavy chain comprising the sequence of SEQ ID NO: 53 and a light chain comprising the sequence of SEQ ID NO: 60. In some of the above compositions, the composition further comprises at least one chemotherapeutic agent.

The disclosure also comprises compositions comprising an anti-GITR antibody and an anti-PD-1 antibody for use in a method of treating cancer according to any one of claims 16-31; wherein the anti-PD-1 antibody is selected from: a) an antibody comprising a heavy chain comprising the sequence of SEQ ID NO: 100 and a light chain comprising the sequence of SEQ ID NO: 102; b) an antibody comprising a heavy chain comprising a heavy chain (HC) complementarity determining region 1 (CDR1) having the sequence of SEQ ID NO: 105, an HC CDR2 having the sequence of SEQ ID NO: 107, and an HC CDR3 having the sequence of SEQ ID NO: 109, and a light chain comprising a light chain (LC) CDR1 having the sequence of SEQ ID NO: 112, a LC CDR2 having the sequence of SEQ ID NO: 114, and a LC CDR3 having the sequence of SEQ ID NO: 116; and c) an antibody comprising a heavy chain comprising the sequences of SEQ ID NOs: 100 and 101 and a light chain comprising the sequences of SEQ ID NOs: 102 and 103; and wherein the anti-GITR antibody is selected from: a) an antibody comprising a GITR binding domain (GITR-BD) comprising a CDR1 comprising the sequence of SEQ ID NO: 120, a CDR2 comprising the sequence of SEQ ID NO: 121, and a CDR3 comprising the sequence of SEQ ID NO: 122; b) an antibody comprising a GITR-BD comprising the sequence of SEQ ID NO: 119; c) a tetravalent molecule comprising two copies of a polypeptide having the structure (GITR-BD)-Linker-(GITR-BD)-Linker-Hinge-Fc, wherein (i) the GITR-BD comprises a CDR1 comprising the sequence of SEQ ID NO: 120, a CDR2 comprising the sequence of SEQ ID NO: 121, and a CDR3 comprising the sequence of SEQ ID NO: 122, (ii) the Linker is a polypeptide, (iii) the Hinge is a polypeptide derived from an immunoglobulin hinge region, and (iv) the Fc is an immunoglobulin Fc polypeptide; d) a tetravalent molecule comprising two copies of a polypeptide having the structure (GITR-BD)-Linker-(GITR-BD)-Linker-Hinge-Fc, wherein (i) the GITR-BD comprises the amino acid sequence of SEQ ID NO:119, (ii) the Linker is a polypeptide, (iii) the Hinge is a polypeptide derived from an immunoglobulin hinge region, and (iv) the Fc is an immunoglobulin Fc polypeptide; and e) a tetravalent molecule comprising two copies of a polypeptide comprising the sequence of SEQ ID NO: 118. In some of the above compositions, the composition further comprises at least one chemotherapeutic agent.

The disclosure further contemplates uses of an anti-GITR antibody for preparation of a medicament for treating cancer in a subject, for example according to the steps and/or conditions of any one of the methods of treatment described above, wherein the anti-GITR antibody is selected from: a) an antibody comprising a GITR binding domain (GITR-BD) comprising a CDR1 comprising the sequence of SEQ ID NO: 120, a CDR2 comprising the sequence of SEQ ID NO: 121, and a CDR3 comprising the sequence of SEQ ID NO: 122; b) an antibody comprising a GITR-BD comprising the sequence of SEQ ID NO: 119; c) a tetravalent molecule comprising two copies of a polypeptide having the structure (GITR-BD)-Linker-(GITR-BD)-Linker-Hinge-Fc, wherein (i) the GITR-BD comprises a CDR1 comprising the sequence of SEQ ID NO: 120, a CDR2 comprising the sequence of SEQ ID NO: 121, and a CDR3 comprising the sequence of SEQ ID NO: 122, (ii) the Linker is a polypeptide, (iii) the Hinge is a polypeptide derived from an immunoglobulin hinge region, and (iv) the Fc is an immunoglobulin Fc polypeptide; d) a tetravalent molecule comprising two copies of a polypeptide having the structure (GITR-BD)-Linker-(GITR-BD)-Linker-Hinge-Fc, wherein (i) the GITR-BD comprises the amino acid sequence of SEQ ID NO:119, (ii) the Linker is a polypeptide, (iii) the Hinge is a polypeptide derived from an immunoglobulin hinge region, and (iv) the Fc is an immunoglobulin Fc polypeptide; and e) a tetravalent molecule comprising two copies of a polypeptide comprising the sequence of SEQ ID NO: 118. In some such uses, the anti-CSF1R antibody is selected from: a) an antibody comprising a heavy chain comprising the sequence of SEQ ID NO: 39 and a light chain comprising the sequence of SEQ ID NO: 46; b) an antibody comprising a heavy chain comprising a heavy chain (HC) complementarity determining region 1 (CDR1) comprising the sequence of SEQ ID NO: 15, an HC CDR2 comprising the sequence of SEQ ID NO: 16, and an HC CDR3 comprising the sequence of SEQ ID NO: 17, and a light chain comprising a light chain (LC) CDR1 comprising the sequence of SEQ ID NO: 18, a LC CDR2 comprising the sequence of SEQ ID NO: 19, and a LC CDR3 comprising the sequence of SEQ ID NO: 20; and c) an antibody comprising a heavy chain comprising the sequence of SEQ ID NO: 53 and a light chain comprising the sequence of SEQ ID NO: 60. In some embodiments, the treatment further comprises administering at least one chemotherapeutic agent.

The disclosure also encompasses uses of the compositions comprising an anti-GITR antibody and an anti-PD-1 antibody for preparation of a medicament for treating cancer in a subject, for example according to the steps and/or conditions of the methods described above, wherein the anti-PD-1 antibody is selected from: a) an antibody comprising a heavy chain comprising the sequence of SEQ ID NO: 100 and a light chain comprising the sequence of SEQ ID NO: 102; b) an antibody comprising a heavy chain comprising a heavy chain (HC) complementarity determining region 1 (CDR1) having the sequence of SEQ ID NO: 105, an HC CDR2 having the sequence of SEQ ID NO: 107, and an HC CDR3 having the sequence of SEQ ID NO: 109, and a light chain comprising a light chain (LC) CDR1 having the sequence of SEQ ID NO: 112, a LC CDR2 having the sequence of SEQ ID NO: 114, and a LC CDR3 having the sequence of SEQ ID NO: 116; and c) an antibody comprising a heavy chain comprising the sequences of SEQ ID NOs: 100 and 101 and a light chain comprising the sequences of SEQ ID NOs: 102 and 103; and wherein the anti-GITR antibody is selected from: a) an antibody comprising a GITR binding domain (GITR-BD) comprising a CDR1 comprising the sequence of SEQ ID NO: 120, a CDR2 comprising the sequence of SEQ ID NO: 121, and a CDR3 comprising the sequence of SEQ ID NO: 122; b) an antibody comprising a GITR-BD comprising the sequence of SEQ ID NO: 119; c) a tetravalent molecule comprising two copies of a polypeptide having the structure (GITR-BD)-Linker-(GITR-BD)-Linker-Hinge-Fc, wherein (i) the GITR-BD comprises a CDR1 comprising the sequence of SEQ ID NO: 120, a CDR2 comprising the sequence of SEQ ID NO: 121, and a CDR3 comprising the sequence of SEQ ID NO: 122, (ii) the Linker is a polypeptide, (iii) the Hinge is a polypeptide derived from an immunoglobulin hinge region, and (iv) the Fc is an immunoglobulin Fc polypeptide; d) a tetravalent molecule comprising two copies of a polypeptide having the structure (GITR-BD)-Linker-(GITR-BD)-Linker-Hinge-Fc, wherein (i) the GITR-BD comprises the amino acid sequence of SEQ ID NO:119, (ii) the Linker is a polypeptide, (iii) the Hinge is a polypeptide derived from an immunoglobulin hinge region, and (iv) the Fc is an immunoglobulin Fc polypeptide; and e) a tetravalent molecule comprising two copies of a polypeptide comprising the sequence of SEQ ID NO: 118. In some embodiments, the treatment further comprises administering at least one chemotherapeutic agent.

The present disclosure also includes methods of treating pancreatic cancer in a subject comprising administering to the subject an anti-Colony Stimulating Factor 1 Receptor (CSF1R) antibody and an anti-Glucocorticoid-Induced TNFR-Related protein (GITR) antibody, wherein the anti-CSF1R antibody is selected from: a) an antibody comprising a heavy chain comprising the sequence of SEQ ID NO: 39 and a light chain comprising the sequence of SEQ ID NO: 46; b) an antibody comprising a heavy chain comprising a heavy chain (HC) complementarity determining region 1 (CDR1) comprising the sequence of SEQ ID NO: 15, an HC CDR2 comprising the sequence of SEQ ID NO: 16, and an HC CDR3 comprising the sequence of SEQ ID NO: 17, and a light chain comprising a light chain (LC) CDR1 comprising the sequence of SEQ ID NO: 18, a LC CDR2 comprising the sequence of SEQ ID NO: 19, and a LC CDR3 comprising the sequence of SEQ ID NO: 20; and c) an antibody comprising a heavy chain comprising the sequence of SEQ ID NO: 53 and a light chain comprising the sequence of SEQ ID NO: 60. In some embodiments, the anti-GITR antibody is selected from: a) an antibody comprising a GITR binding domain (GITR-BD) comprising a CDR1 comprising the sequence of SEQ ID NO: 120, a CDR2 comprising the sequence of SEQ ID NO: 121, and a CDR3 comprising the sequence of SEQ ID NO: 122; b) an antibody comprising a GITR-BD comprising the sequence of SEQ ID NO: 119; c) a tetravalent molecule comprising two copies of a polypeptide having the structure (GITR-BD)-Linker-(GITR-BD)-Linker-Hinge-Fc, wherein (i) the GITR-BD comprises a CDR1 comprising the sequence of SEQ ID NO: 120, a CDR2 comprising the sequence of SEQ ID NO: 121, and a CDR3 comprising the sequence of SEQ ID NO: 122, (ii) the Linker is a polypeptide, (iii) the Hinge is a polypeptide derived from an immunoglobulin hinge region, and (iv) the Fc is an immunoglobulin Fc polypeptide; d) a tetravalent molecule comprising two copies of a polypeptide having the structure (GITR-BD)-Linker-(GITR-BD)-Linker-Hinge-Fc, wherein (i) the GITR-BD comprises the amino acid sequence of SEQ ID NO:119, (ii) the Linker is a polypeptide, (iii) the Hinge is a polypeptide derived from an immunoglobulin hinge region, and (iv) the Fc is an immunoglobulin Fc polypeptide; and e) a tetravalent molecule comprising two copies of a polypeptide comprising the sequence of SEQ ID NO: 118.

The disclosure also encompasses methods of treating pancreatic cancer in a subject comprising administering to the subject an anti-Colony Stimulating Factor 1 Receptor (CSF1R) antibody and an anti-Glucocorticoid-Induced TNFR-Related protein (GITR) antibody, wherein the anti-GITR antibody is a tetravalent molecule having the structure (GITR-BD)-Linker-(GITR-BD)-Linker-Hinge-Fc, wherein (i) the GITR-BD comprises (a) a CDR1 comprising the sequence of SEQ ID NO: 120, a CDR2 comprising the sequence of SEQ ID NO: 121, and a CDR3 comprising the sequence of SEQ ID NO: 122 or (b) the sequence of SEQ ID NO:119, (ii) the Linker is a polypeptide comprising a sequence selected from SEQ ID NOs: 134-140, (iii) the Hinge is a polypeptide comprising a sequence selected from SEQ ID NOs: 129-133, and (iv) the Fc is an immunoglobulin Fc polypeptide comprising a sequence selected from SEQ ID NOs: 123-128. In some such methods, the anti-CSF1R antibody is selected from: a) an antibody comprising a heavy chain comprising the sequence of SEQ ID NO: 39 and a light chain comprising the sequence of SEQ ID NO: 46; b) an antibody comprising a heavy chain comprising a heavy chain (HC) complementarity determining region 1 (CDR1) comprising the sequence of SEQ ID NO: 15, an HC CDR2 comprising the sequence of SEQ ID NO: 16, and an HC CDR3 comprising the sequence of SEQ ID NO: 17, and a light chain comprising a light chain (LC) CDR1 comprising the sequence of SEQ ID NO: 18, a LC CDR2 comprising the sequence of SEQ ID NO: 19, and a LC CDR3 comprising the sequence of SEQ ID NO: 20; and c) an antibody comprising a heavy chain comprising the sequence of SEQ ID NO: 53 and a light chain comprising the sequence of SEQ ID NO: 60.

In any of the pancreatic cancer treatment methods above, the anti-CSF1R antibody may be a humanized antibody or is selected from a Fab, an Fv, an scFv, a Fab′, and a (Fab′)₂. In some embodiments, the anti-CSF1R antibody and the anti-GITR antibody are administered concurrently or sequentially. In some embodiments, the anti-CSF1R antibody and the anti-GITR antibody are administered once every week, once every 2 weeks, once every 3 weeks, once every 4 weeks, or once every 5 weeks. In some embodiments, the anti-CSF1R antibody is administered at a dose of 0.1, 0.3, 0.5, 1, 2, 3, 4, 5, or 10 mg/kg. In some such embodiments, the anti-CSF1R antibody is administered at a dose of 1, 2, 3, or 4 mg/kg every 2 weeks or every 3 weeks. In some embodiments, the anti-CSF1R antibody blocks binding of both CSF1 and IL-34 to CSF1R. In some embodiments, the anti-CSF1R antibody inhibits ligand-induced CSF1R phosphorylation in vitro. In some embodiments, administration of the anti-CSF1R antibody and the anti-GITR antibody results in a synergistic effect. In some embodiments, administration of the anti-CSF1R antibody and the anti-GITR antibody results in a synergistic inhibition of tumor growth in a mouse xenograft or syngeneic pancreatic cancer model. In some embodiments, the method further comprises administering at least one chemotherapeutic agent. In some such embodiments, the at least one chemotherapeutic agent is selected from gemcitabine, nab-pactlitaxel, leukovorin (folinic acid), 5-fluorouracil, irinotecan, and oxaliplatin. In some such embodiments, the at least one chemotherapeutic agent is selected from (a) gemcitabine (b) gemcitabine and nab-paclitaxel, and (c) FOLFIRINOX. In some such embodiments, the at least one chemotherapeutic agent is gemcitabine.

In some embodiments of the pancreatic cancer treatment methods, the methods further comprise administering an anti-PD-1 antibody. In some cases, the anti-PD-1 antibody is selected from: a) an antibody comprising a heavy chain comprising the sequence of SEQ ID NO: 100 and a light chain comprising the sequence of SEQ ID NO: 102; b) an antibody comprising a heavy chain comprising a heavy chain (HC) complementarity determining region 1 (CDR1) having the sequence of SEQ ID NO: 105, an HC CDR2 having the sequence of SEQ ID NO: 107, and an HC CDR3 having the sequence of SEQ ID NO: 109, and a light chain comprising a light chain (LC) CDR1 having the sequence of SEQ ID NO: 112, a LC CDR2 having the sequence of SEQ ID NO: 114, and a LC CDR3 having the sequence of SEQ ID NO: 116; and c) an antibody comprising a heavy chain comprising the sequences of SEQ ID NOs: 100 and 101 and a light chain comprising the sequences of SEQ ID NOs: 102 and 103.

The present disclosure also encompasses methods of treating pancreatic cancer in a subject comprising administering to the subject an anti-Colony Stimulating Factor 1 Receptor (CSF1R) antibody, an anti-Glucocorticoid-Induced TNFR-Related protein (GITR) antibody, and at least one chemotherapeutic agent selected from gemcitabine, nab-pactlitaxel, leukovorin (folinic acid), 5-fluorouracil, irinotecan, and oxaliplatin. In some embodiments, the at least one chemotherapeutic agent is selected from (a) gemcitabine, (b) gemcitabine and nab-paclitaxel, and (c) FOLFIRINOX. In some embodiments, the at least one chemotherapeutic agent is gemcitabine. In some embodiments, the anti-GITR antibody is selected from: a) an antibody comprising a GITR binding domain (GITR-BD) comprising a CDR1 comprising the sequence of SEQ ID NO: 120, a CDR2 comprising the sequence of SEQ ID NO: 121, and a CDR3 comprising the sequence of SEQ ID NO: 122; b) an antibody comprising a GITR-BD comprising the sequence of SEQ ID NO: 119; c) a tetravalent molecule comprising two copies of a polypeptide having the structure (GITR-BD)-Linker-(GITR-BD)-Linker-Hinge-Fc, wherein (i) the GITR-BD comprises a CDR1 comprising the sequence of SEQ ID NO: 120, a CDR2 comprising the sequence of SEQ ID NO: 121, and a CDR3 comprising the sequence of SEQ ID NO: 122, (ii) the Linker is a polypeptide, (iii) the Hinge is a polypeptide derived from an immunoglobulin hinge region, and (iv) the Fc is an immunoglobulin Fc polypeptide; d) a tetravalent molecule comprising two copies of a polypeptide having the structure (GITR-BD)-Linker-(GITR-BD)-Linker-Hinge-Fc, wherein (i) the GITR-BD comprises the amino acid sequence of SEQ ID NO:119, (ii) the Linker is a polypeptide, (iii) the Hinge is a polypeptide derived from an immunoglobulin hinge region, and (iv) the Fc is an immunoglobulin Fc polypeptide; and e) a tetravalent molecule comprising two copies of a polypeptide comprising the sequence of SEQ ID NO: 118. In some embodiments, the anti-GITR antibody is a tetravalent molecule having the structure (GITR-BD)-Linker-(GITR-BD)-Linker-Hinge-Fc, wherein (i) the GITR-BD comprises (a) a CDR1 comprising the sequence of SEQ ID NO: 120, a CDR2 comprising the sequence of SEQ ID NO: 121, and a CDR3 comprising the sequence of SEQ ID NO: 122 or (b) the sequence of SEQ ID NO:119, (ii) the Linker is a polypeptide comprising a sequence selected from SEQ ID NOs: 134-140, (iii) the Hinge is a polypeptide comprising a sequence selected from SEQ ID NOs: 129-133, and (iv) the Fc is an immunoglobulin Fc polypeptide comprising a sequence selected from SEQ ID NOs: 123-128. In some such methods, the anti-CSF1R antibody is selected from: a) an antibody comprising a heavy chain comprising the sequence of SEQ ID NO: 39 and a light chain comprising the sequence of SEQ ID NO: 46; b) an antibody comprising a heavy chain comprising a heavy chain (HC) complementarity determining region 1 (CDR1) comprising the sequence of SEQ ID NO: 15, an HC CDR2 comprising the sequence of SEQ ID NO: 16, and an HC CDR3 comprising the sequence of SEQ ID NO: 17, and a light chain comprising a light chain (LC) CDR1 comprising the sequence of SEQ ID NO: 18, a LC CDR2 comprising the sequence of SEQ ID NO: 19, and a LC CDR3 comprising the sequence of SEQ ID NO: 20; and c) an antibody comprising a heavy chain comprising the sequence of SEQ ID NO: 53 and a light chain comprising the sequence of SEQ ID NO: 60. In some cases, the method further comprises administering an anti-PD-1 antibody. And in some such embodiments, the anti-PD-1 antibody is selected from: a) an antibody comprising a heavy chain comprising the sequence of SEQ ID NO: 100 and a light chain comprising the sequence of SEQ ID NO: 102; b) an antibody comprising a heavy chain comprising a heavy chain (HC) complementarity determining region 1 (CDR1) having the sequence of SEQ ID NO: 105, an HC CDR2 having the sequence of SEQ ID NO: 107, and an HC CDR3 having the sequence of SEQ ID NO: 109, and a light chain comprising a light chain (LC) CDR1 having the sequence of SEQ ID NO: 112, a LC CDR2 having the sequence of SEQ ID NO: 114, and a LC CDR3 having the sequence of SEQ ID NO: 116; and c) an antibody comprising a heavy chain comprising the sequences of SEQ ID NOs: 100 and 101 and a light chain comprising the sequences of SEQ ID NOs: 102 and 103.

The present disclosure further contemplates compositions comprising an anti-GITR antibody for use in a method of treating pancreatic cancer according to any one of the pancreatic cancer treatment methods described above. In some composition embodiments, the anti-GITR antibody is selected from: a) an antibody comprising a GITR binding domain (GITR-BD) comprising a CDR1 comprising the sequence of SEQ ID NO: 120, a CDR2 comprising the sequence of SEQ ID NO: 121, and a CDR3 comprising the sequence of SEQ ID NO: 122; b) an antibody comprising a GITR-BD comprising the sequence of SEQ ID NO: 119; c) a tetravalent molecule comprising two copies of a polypeptide having the structure (GITR-BD)-Linker-(GITR-BD)-Linker-Hinge-Fe, wherein (i) the GITR-BD comprises a CDR1 comprising the sequence of SEQ ID NO: 120, a CDR2 comprising the sequence of SEQ ID NO: 121, and a CDR3 comprising the sequence of SEQ ID NO: 122, (ii) the Linker is a polypeptide, (iii) the Hinge is a polypeptide derived from an immunoglobulin hinge region, and (iv) the Fc is an immunoglobulin Fc polypeptide; d) a tetravalent molecule comprising two copies of a polypeptide having the structure (GITR-BD)-Linker-(GITR-BD)-Linker-Hinge-Fc, wherein (i) the GITR-BD comprises the amino acid sequence of SEQ ID NO:119, (ii) the Linker is a polypeptide, (iii) the Hinge is a polypeptide derived from an immunoglobulin hinge region, and (iv) the Fc is an immunoglobulin Fc polypeptide; and e) a tetravalent molecule comprising two copies of a polypeptide comprising the sequence of SEQ ID NO: 118. In some embodiments, the anti-GITR antibody is a tetravalent molecule having the structure (GITR-BD)-Linker-(GITR-BD)-Linker-Hinge-Fc, wherein (i) the GITR-BD comprises (a) a CDR1 comprising the sequence of SEQ ID NO: 120, a CDR2 comprising the sequence of SEQ ID NO: 121, and a CDR3 comprising the sequence of SEQ ID NO: 122 or (b) the sequence of SEQ ID NO:119, (ii) the Linker is a polypeptide comprising a sequence selected from SEQ ID NOs: 134-140, (iii) the Hinge is a polypeptide comprising a sequence selected from SEQ ID NOs: 129-133, and (iv) the Fc is an immunoglobulin Fc polypeptide comprising a sequence selected from SEQ ID NOs: 123-128.

The disclosure further contemplates uses of compositions comprising an anti-GITR antibody and an anti-CSF1R antibody for preparation of a medicament for treating pancreatic cancer in a subject according to the steps and/or conditions of any one of the pancreatic cancer treatment methods above. In some such use embodiments, the anti-GITR antibody is selected from: a) an antibody comprising a GITR binding domain (GITR-BD) comprising a CDR1 comprising the sequence of SEQ ID NO: 120, a CDR2 comprising the sequence of SEQ ID NO: 121, and a CDR3 comprising the sequence of SEQ ID NO: 122; b) an antibody comprising a GITR-BD comprising the sequence of SEQ ID NO: 119; c) a tetravalent molecule comprising two copies of a polypeptide having the structure (GITR-BD)-Linker-(GITR-BD)-Linker-Hinge-Fc, wherein (i) the GITR-BD comprises a CDR1 comprising the sequence of SEQ ID NO: 120, a CDR2 comprising the sequence of SEQ ID NO: 121, and a CDR3 comprising the sequence of SEQ ID NO: 122, (ii) the Linker is a polypeptide, (iii) the Hinge is a polypeptide derived from an immunoglobulin hinge region, and (iv) the Fc is an immunoglobulin Fc polypeptide; d) a tetravalent molecule comprising two copies of a polypeptide having the structure (GITR-BD)-Linker-(GITR-BD)-Linker-Hinge-Fc, wherein (i) the GITR-BD comprises the amino acid sequence of SEQ ID NO:119, (ii) the Linker is a polypeptide, (iii) the Hinge is a polypeptide derived from an immunoglobulin hinge region, and (iv) the Fc is an immunoglobulin Fc polypeptide; and e) a tetravalent molecule comprising two copies of a polypeptide comprising the sequence of SEQ ID NO: 118. In some embodiments, the anti-GITR antibody is a tetravalent molecule having the structure (GITR-BD)-Linker-(GITR-BD)-Linker-Hinge-Fc, wherein (i) the GITR-BD comprises (a) a CDR1 comprising the sequence of SEQ ID NO: 120, a CDR2 comprising the sequence of SEQ ID NO: 121, and a CDR3 comprising the sequence of SEQ ID NO: 122 or (b) the sequence of SEQ ID NO:119, (ii) the Linker is a polypeptide comprising a sequence selected from SEQ ID NOs: 134-140, (iii) the Hinge is a polypeptide comprising a sequence selected from SEQ ID NOs: 129-133, and (iv) the Fc is an immunoglobulin Fc polypeptide comprising a sequence selected from SEQ ID NOs: 123-128. In some such uses, the anti-CSF1R antibody is selected from: a) an antibody comprising a heavy chain comprising the sequence of SEQ ID NO: 39 and a light chain comprising the sequence of SEQ ID NO: 46; b) an antibody comprising a heavy chain comprising a heavy chain (HC) complementarity determining region 1 (CDR1) comprising the sequence of SEQ ID NO: 15, an HC CDR2 comprising the sequence of SEQ ID NO: 16, and an HC CDR3 comprising the sequence of SEQ ID NO: 17, and a light chain comprising a light chain (LC) CDR1 comprising the sequence of SEQ ID NO: 18, a LC CDR2 comprising the sequence of SEQ ID NO: 19, and a LC CDR3 comprising the sequence of SEQ ID NO: 20; and c) an antibody comprising a heavy chain comprising the sequence of SEQ ID NO: 53 and a light chain comprising the sequence of SEQ ID NO: 60.

### BRIEF DESCRIPTION OF THE FIGURES

FIG. 1A-C show an alignment of the humanized heavy chain variable regions for each of anti-CSF1R humanized antibodies huAb1 to huAb16. Boxed residues are amino acids in the human acceptor sequence that were changed back to the corresponding mouse residue.

FIG. 2A-C show an alignment of the humanized light chain variable regions for each of humanized anti-CSF1R antibodies huAb1 to huAb16. Boxed amino acids are residues in the human acceptor sequence that were changed back to the corresponding mouse residue.

FIGS. 3A-3G provide schematic representations of several different exemplary anti-GITR antibody architectures.

FIG. 4A shows changes in tumor volume in an MC38 murine tumor model in the presence of a murine IgG2a control antibody, an anti-CSF1R antibody, an anti-GITR antibody, and a combination of anti-CSF1R and anti-GITR. FIG. 4B shows tumor volume in individual mice in the IgG2a control, anti-CSF1R, anti-GITR, and anti-CSF1R/anti-GITR groups at day 24 post-inoculation in the MC38 tumor model. Tumor volume at day 24 was significantly lower in the anti-CSF1R/anti-GITR group compared to either the anti-CSF1R group (P=0.0029) or the anti-GITR group (P=0.0376).

FIG. 5A-5D show changes in tumor volume in days post-inoculation in a murine MC38 tumor model for individual mice given a murine IgG2a control (FIG. 5A), a tetravalent anti-GITR antibody with a wild type murine Fc IgG2a sequence (tetravalent llama C06-mIgG2a; FIG. 5B), a tetravalent anti-GITR antibody with a mutant murine Fc IgG2a sequence intended to reduce Fc functions (tetravalent llama C06-mIgG2a Fc silent; FIG. 5C), and an anti-PD-1 antibody (FIG. 5D). FIG. 5E shows changes tumor volume in days post-inoculation for individual mice given a combination of anti-PD-1 antibody and anti-GITR antibody with wild type murine Fc (tetravalent llama C06-mIgG2a+anti-PD-1). FIG. 5F shows changes in tumor volume in days post-inoculation for individual mice given a combination of anti-PD-1 antibody and anti-GITR antibody with mutant murine Fc (tetravalent llama C06-mIgG2a Fc silent+anti-PD-1).

FIG. 6 shows the percent survival of C57BL/6 mice inoculated surgically with KRas^{G12D}/p53^{-/-} murine pancreatic ductal adenocarincoma (PDAC) cells after treatment beginning on day 13 post-inoculation (downward arrows show administrations of each drug) with an IgG control, a combination of an anti-GITR antibody and gemcitabine (GEM), or a combination of the anti-GITR antibody, an anti-CSF IR antibody, and GEM. As discussed further in Example 3 below, treatment with anti-GITR antibody and GEM significantly increased the survival of PDAC tumor-bearing mice compared to the IgG control to a median of 34 days compared to 26 days (p<0.001). However, survival of animals treated with the combination of anti-GITR and anti-CSF1R antibodies plus GEM was significantly higher, a median of 40 days with p<0.05 compared to the anti-GITR plus GEM group and p<0.0001 compared to the IgG control group. P-values were calculated using the Log-rank (Mantel-Cox) test comparing individual treatment groups.

### DETAILED DESCRIPTION

In some embodiments, this disclosure provides methods of treating tumors that may be sensitive to combination therapy with an anti-GITR antibody and an anti-PD-1 antibody. In some embodiments, this disclosure provides methods of treating tumors that may be sensitive to combination therapy with an anti-GITR antibody and an anti-CSF1R antibody. In some embodiments, this disclosure provides methods of treating tumors that may be sensitive to combination therapy with all three of an anti-GITR antibody, an anti-PD-1 antibody, and an anti-CSF1R antibody. In some instances, tumors that have both CSF1R-expressing tumor-associated macrophages (TAMs) and PD-1-expressing CD8+ T cells may be resistant to PD-1/PD-L1 monotherapy, but may be sensitive to one of the above combination therapies.

The section headings used herein are for organizational purposes only and are not to be construed as limiting the subject matter described. All references cited herein, including patent applications and publications, are incorporated herein by reference in their entireties for any purpose.

### Definitions

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular.

Exemplary techniques used in connection with recombinant DNA, oligonucleotide synthesis, tissue culture and transformation (e.g., electroporation, lipofection), enzymatic reactions, and purification techniques are known in the art. Many such techniques and procedures are described, e.g., in Sambrook et al. Molecular Cloning: A Laboratory Manual (2nd ed., Cold Spring Harbor Laboratory Press, Cold Spring Harbor, N.Y. (1989)), among other places. In addition, exemplary techniques for chemical syntheses, chemical analyses, pharmaceutical preparation, formulation, and delivery, and treatment of patients are also known in the art.

In this application, the use of “or” means “and/or” unless stated otherwise. In the context of a multiple dependent claim, the use of “or” refers back to more than one preceding independent or dependent claim in the alternative only. Also, terms such as “element” or “component” encompass both elements and components comprising one unit and elements and components that comprise more than one subunit unless specifically stated otherwise.

As described herein, any concentration range, percentage range, ratio range or integer range is to be understood to include the value of any integer within the recited range and, when appropriate, fractions thereof (such as one tenth and one hundredth of an integer), unless otherwise indicated.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

As utilized in accordance with the present disclosure, the following terms, unless otherwise indicated, shall be understood to have the following meanings:

“Administering” refers to the physical introduction of a composition comprising a therapeutic agent to a subject, using any of the various methods and delivery systems known to those skilled in the art. Routes of administration for antibodies disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intratumoral, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. Non-parenteral routes include a topical, epidermal or mucosal route of administration, for example, orally, intranasally, vaginally, rectally, sublingually or topically. Administering can also be performed, for example, once, a plurality of times, and/or over one or more extended periods.

The terms “nucleic acid molecule” and “polynucleotide” may be used interchangeably, and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides that comprise the nucleic acid molecule or polynucleotide.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present invention, a “polypeptide” refers to a protein that includes modifications, such as deletions, additions, and substitutions (generally conservative in nature), to a native sequence, as long as the protein maintains the desired activity. These modifications may be deliberate, as through site-directed mutagenesis, or may be accidental, such as through mutations of hosts that produce the proteins or errors due to PCR amplification.

As used herein, whether a particular amino acid sequence is, for example, at least 95% identical to a specific reference sequence can be determined using, e.g., a computer program. When determining whether a particular sequence is, for example, 95% identical to a reference sequence, the percentage of identity is calculated over the full length of the reference amino acid sequence.

The term “GITR” refers herein to the full-length, mature, human GITR protein, except where specifically noted otherwise (i.e. “murine GITR” or a GITR fragment or domain, etc.).

The term “anti-GITR antibody” refers herein to an antibody molecule that binds GITR and thereby acts as an agonist to activate GITR signaling. For example, in some embodiments, the anti-GITR antibody may block binding between GITR and its ligand GITRL.

In some embodiments herein, an anti-GITR antibody comprises a “fusion” polypeptide. A “fusion” polypeptide indicates a chimeric polypeptide molecule that may be formed by joining together amino acid sequences from two different polypeptide molecules whose amino acid sequences would not be joined together in nature, such as a single domain antibody or an antibody heavy chain variable region from one species and an Fc polypeptide or other antibody constant region of a different species.

The term “CSF1R” refers herein to the full-length human CSF1R, which includes the N-terminal ECD, the transmembrane domain, and the intracellular tyrosine kinase domain, with or without an N-terminal leader sequence, unless specifically indicated otherwise (i.e. “murine CSF1R”).

The term “anti-CSF1R antibody” refers to an antibody molecule that binds CSF1R and thereby blocks binding of CSF1R to one or both of its ligands CSF1 and IL-34.

The terms “programmed cell death protein 1” and abbreviations “PD-1” and “PD1” refer to the full-length, mature human PD-1 protein, which is an immunoinhibitory receptor belonging to the CD28 family.

The terms “programmed cell death 1 ligand 1” and “PD-L1” (PD-L1; B7 homolog-1; B7-H1; or CD274) and “Programmed Death Ligand-2” (PD-L2; B7-DC; or CD273) are two cell surface glycoprotein ligands for PD-1 that downregulate T-cell activation and cytokine secretion upon binding to PD-1. The term “PD-L1” as used herein refers to full-length, mature, human PD-L1 unless specifically noted otherwise.

“Cytotoxic T-Lymphocyte Antigen-4” (CTLA-4) refers to an immunoinhibitory receptor belonging to the CD28 family. CTLA-4 is expressed exclusively on T cells in vivo, and binds to two ligands, CD80 and CD86 (also called B7-1 and B7-2, respectively). The term “CTLA-4” as used herein refers to full-length, mature, human CTLA-4 unless specifically noted otherwise.

The term “anti-PD-1 antibody” or “anti-PD1 antibody” refers to an antibody that binds to PD-1 and thereby inhibits PD-1 and/or PD-L1 signaling. In some embodiments, the antibody binds to PD-1 and blocks binding of PD-L1 and/or PD-L2 to PD-1.

The term “PD-1/PD-L1 inhibitor” refers to a moiety that disrupts the PD-1/PD-L1 signaling pathway. In some embodiments, the inhibitor inhibits the PD-1/PD-L1 signaling pathway by binding to PD-1 and/or PD-L1. In some embodiments, the inhibitor also binds to PD-L2. In some embodiments, a PD-1/PD-L1 inhibitor blocks binding of PD-1 to PD-L1 and/or PD-L2.

As used herein, antibodies may “block binding of” their target GITR, CSF1R, or PD-1 to one or more of its ligands, meaning that they have the ability to inhibit interaction between a target and ligand (e.g., between CSF1R and CSF1 and/or IL-34 in the case of anti-CSF1R antibodies or between PD-1 and PD-L1 and/or PD-L2 in the case of anti-PD-1 antibodies). Such inhibition may occur through any mechanism, including direct interference with ligand binding, e.g., because of overlapping binding sites on the target protein for the antibody and ligand, and/or due to conformational changes induced by antibody binding that alter ligand affinity, etc. Antibodies and antibody fragments referred to as “functional” are characterized by having such properties.

The term “antibody” as used herein refers to a molecule comprising at least complementarity-determining region (CDR) 1, CDR2, and CDR3 of a single domain antibody (sdAb), wherein the molecule is capable of binding to antigen. The term antibody also refers to molecules comprising at least CDR1, CDR2, and CDR3 of a heavy chain and CDR1, CDR2, and CDR3 of a light chain, wherein the molecule is capable of binding to antigen. The term antibody also includes fragments that are capable of binding antigen, such as Fv, single-chain Fv (scFv), Fab, Fab′, and (Fab′)₂. The term antibody also includes chimeric antibodies, humanized antibodies, and antibodies of various species such as mouse, human, cynomolgus monkey, llama, camel, etc. The term also includes multivalent antibodies such as bivalent or tetravalent antibodies. A multivalent antibody includes, e.g., a single polypeptide chain comprising multiple antigen binding (CDR-containing) domains, as well as two or more polypeptide chains, each containing one or more antigen binding domains, such two or more polypeptide chains being associated with one another, e.g., through a hinge region capable of forming disulfide bond(s) or any other covalent or noncovalent interaction.

The term “single domain antibody” or “sdAb” as used herein, refers to an antibody molecule or antigen binding fragment thereof comprising a single antigen binding domain sequence comprising a CDR1, CDR2, and CDR3, wherein the sdAb is capable of binding to antigen. Single domain antibodies may be derived from dromedary species, such as llama, camel, and alpaca, or from fish species. Alternatively, single domain antibodies may be obtained by laboratory techniques such as selection methods. In some embodiments, a sdAb may be humanized. In some embodiments, a sdAb may comprise part of a chimeric antibody or multivalent antibody.

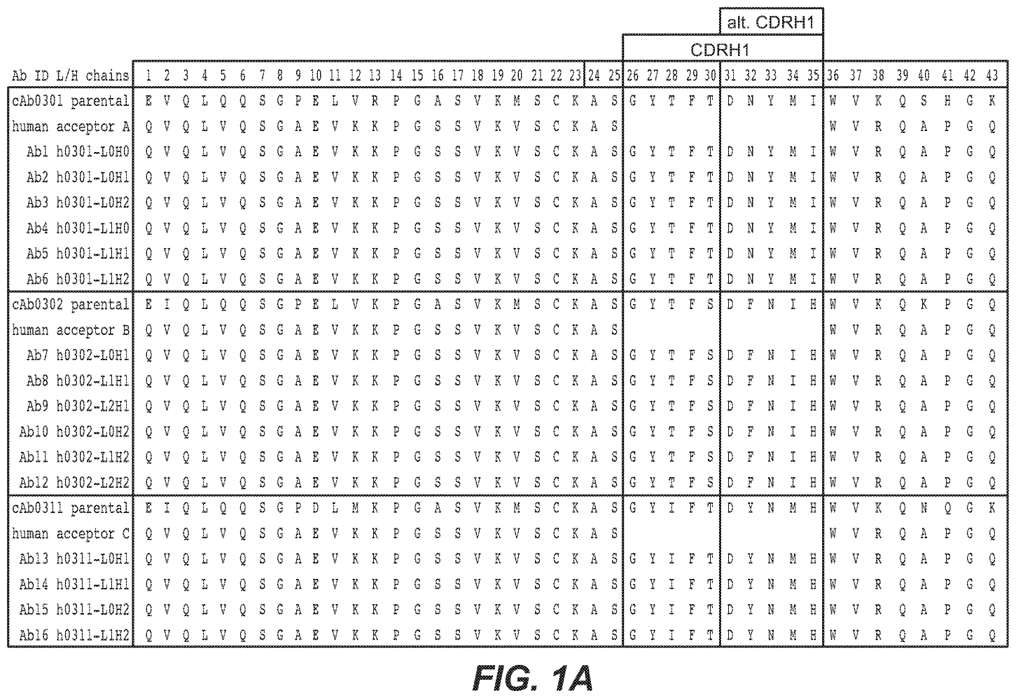

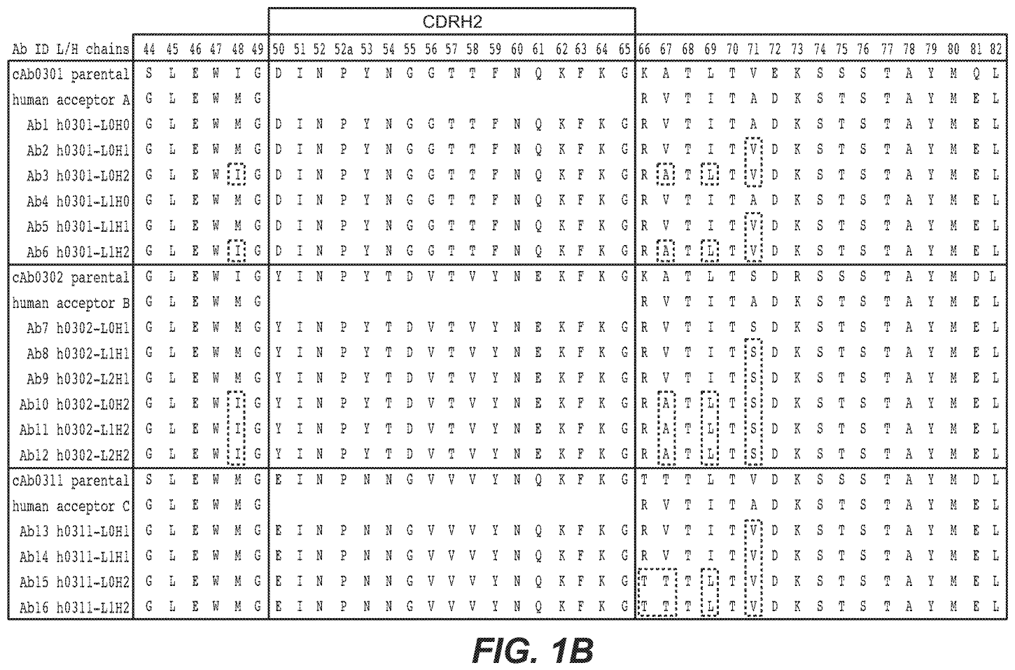

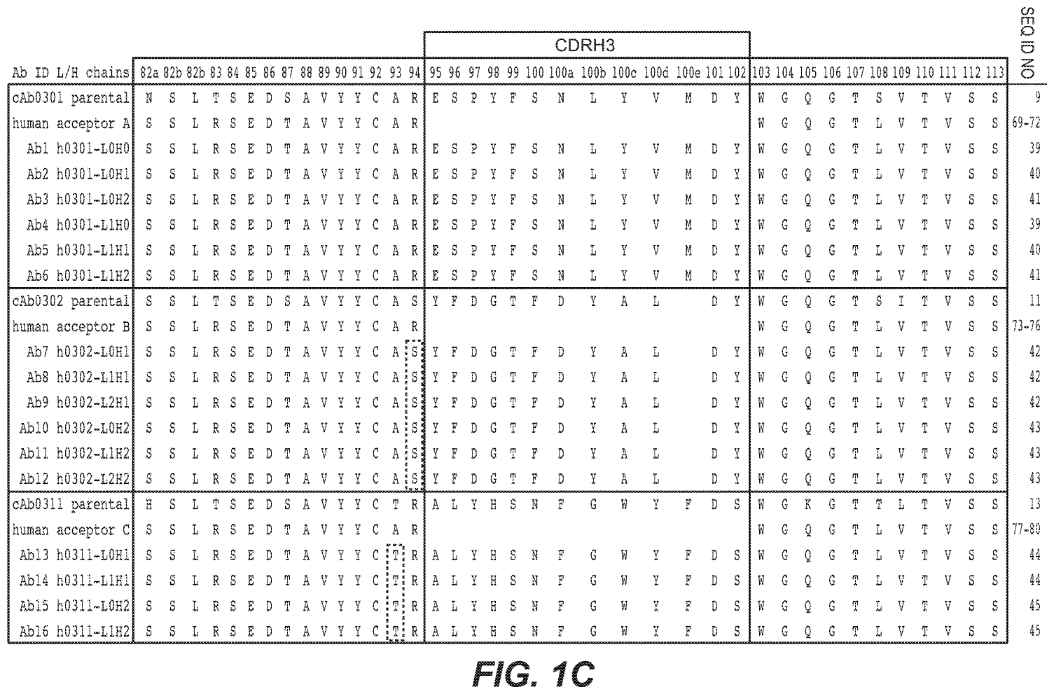

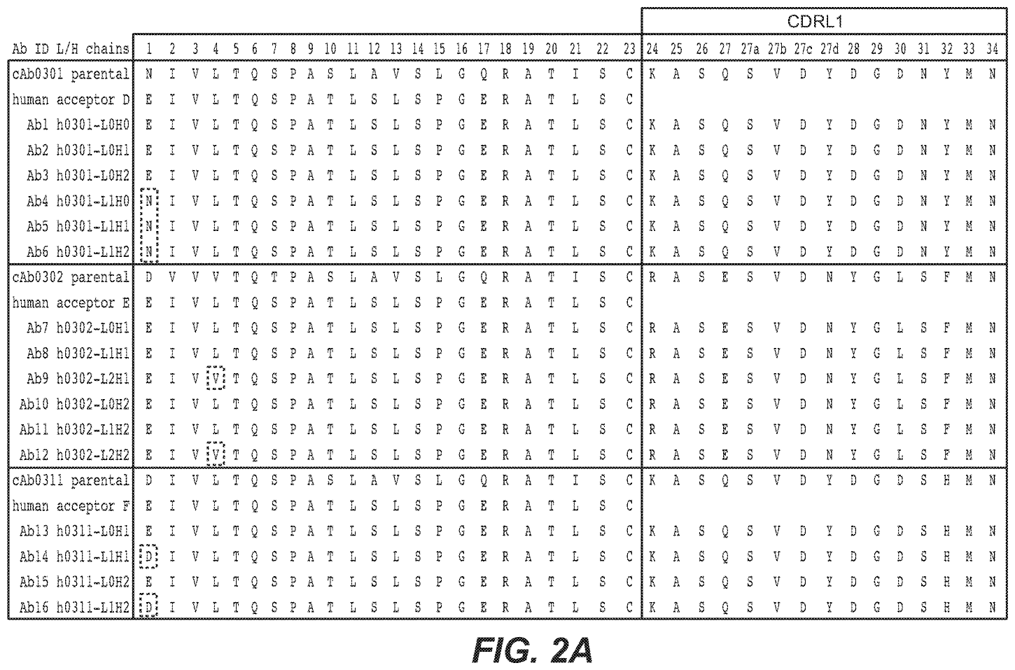

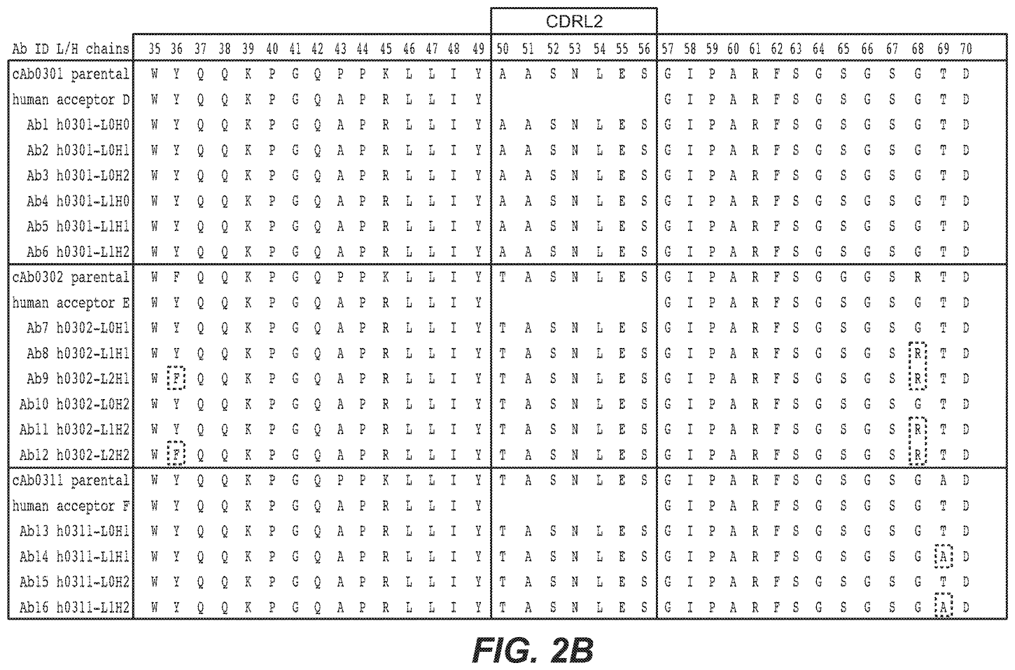

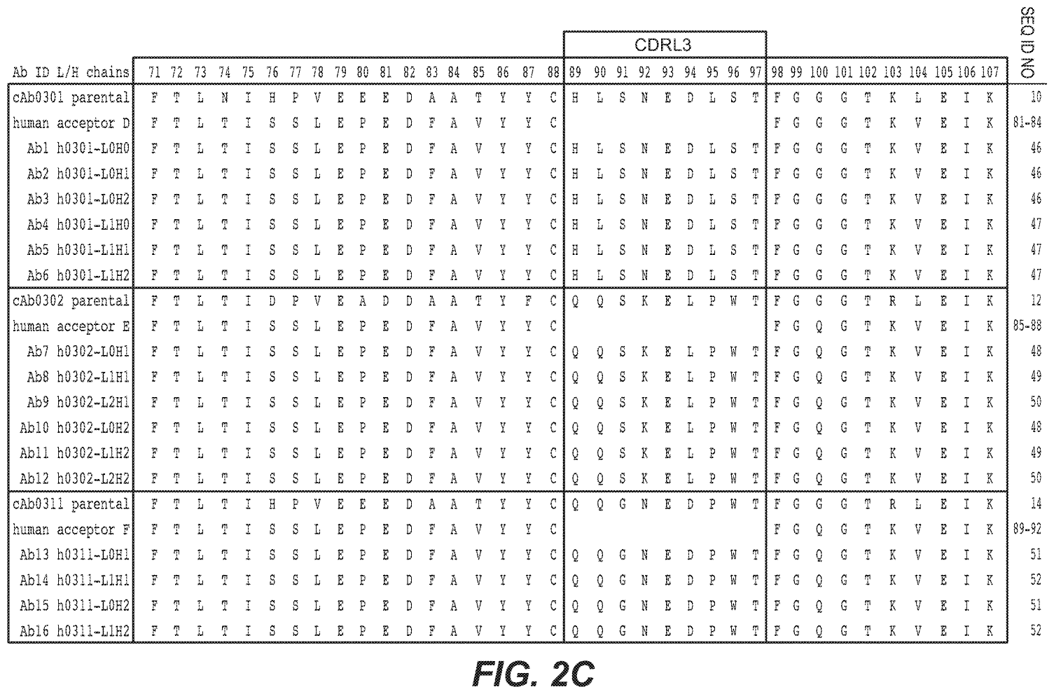

The term “heavy chain variable region” as used herein refers to a region comprising heavy chain CDR1, framework (FR) 2, CDR2, FR3, and CDR3. In some embodiments, a heavy chain variable region also comprises at least a portion of an FR1 and/or at least a portion of an FR4. In some embodiments, a heavy chain CDR1 corresponds to Kabat residues 26 to 35; a heavy chain CDR2 corresponds to Kabat residues 50 to 65; and a heavy chain CDR3 corresponds to Kabat residues 95 to 102. See, e.g., Kabat Sequences of Proteins of Immunological Interest (1987 and 1991, NIH, Bethesda, Md.); and FIG. 1. In some embodiments, a heavy chain CDR1 corresponds to Kabat residues 31 to 35; a heavy chain CDR2 corresponds to Kabat residues 50 to 65; and a heavy chain CDR3 corresponds to Kabat residues 95 to 102. See id.

The term “heavy chain constant region” as used herein refers to a region comprising at least three heavy chain constant domains, C_{H}1, CH₂, and C_{H}3. Nonlimiting exemplary heavy chain constant regions include γ, δ, and α. Nonlimiting exemplary heavy chain constant regions also include ε and μ. Each heavy constant region corresponds to an antibody isotype. For example, an antibody comprising a γ constant region is an IgG antibody, an antibody comprising a δ constant region is an IgD antibody, and an antibody comprising an a constant region is an IgA antibody. Further, an antibody comprising a μ constant region is an IgM antibody, and an antibody comprising an constant region is an IgE antibody. Certain isotypes can be further subdivided into subclasses. For example, IgG antibodies include, but are not limited to, IgG1 (comprising a γ₁ constant region), IgG2 (comprising a γ₂ constant region), IgG3 (comprising a γ₃ constant region), and IgG4 (comprising a γ₄ constant region) antibodies; IgA antibodies include, but are not limited to, IgA1 (comprising an al constant region) and IgA2 (comprising an α₂ constant region) antibodies; and IgM antibodies include, but are not limited to, IgM1 and IgM2.

The term “heavy chain” (abbreviated HC) as used herein refers to a polypeptide comprising at least a heavy chain variable region, with or without a leader sequence. In some embodiments, a heavy chain comprises at least a portion of a heavy chain constant region. The term “full-length heavy chain” as used herein refers to a polypeptide comprising a heavy chain variable region and a heavy chain constant region, with or without a leader sequence.

The term “light chain variable region” as used herein refers to a region comprising light chain CDR1, framework (FR)2, CDR2, FR3, and CDR3. In some embodiments, a light chain variable region also comprises an FR1 and/or an FR4. In some embodiments, a light chain CDR1 corresponds to Kabat residues 24 to 34; a light chain CDR2 corresponds to Kabat residues 50 to 56; and a light chain CDR3 corresponds to Kabat residues 89 to 97. See, e.g., Kabat Sequences of Proteins of Immunological Interest (1987 and 1991, NIH, Bethesda, Md.); and FIG. 1.

The term “light chain constant region” as used herein refers to a region comprising a light chain constant domain, C_{L}. Nonlimiting exemplary light chain constant regions include λ and κ.

The term “light chain” (abbreviate LC) as used herein refers to a polypeptide comprising at least a light chain variable region, with or without a leader sequence. In some embodiments, a light chain comprises at least a portion of a light chain constant region. The term “full-length light chain” as used herein refers to a polypeptide comprising a light chain variable region and a light chain constant region, with or without a leader sequence.

A “chimeric antibody” as used herein refers to an antibody comprising at least one variable region from a first species (such as mouse, rat, cynomolgus monkey, etc.) and at least one constant region from a second species (such as human, cynomolgus monkey, etc.). In some embodiments, a chimeric antibody comprises at least one mouse variable region and at least one human constant region. In some embodiments, a chimeric antibody comprises at least one cynomolgus variable region and at least one human constant region. In some embodiments, a chimeric antibody comprises at least one rat variable region and at least one mouse constant region. In some embodiments, all of the variable regions of a chimeric antibody are from a first species and all of the constant regions of the chimeric antibody are from a second species.

A “humanized antibody” as used herein refers to an antibody in which at least one amino acid in a framework region of a non-human variable region has been replaced with the corresponding amino acid from a human variable region. In some embodiments, a humanized antibody comprises at least one human constant region or fragment thereof. In some embodiments, a humanized antibody is a sdAb, a Fab, an scFv, a (Fab′)₂, etc.

A “CDR-grafted antibody” as used herein refers to a humanized antibody in which the complementarity determining regions (CDRs) of a first (non-human) species have been grafted onto the framework regions (FRs) of a second (human) species.

A “human antibody” as used herein refers to antibodies produced in humans, antibodies produced in non-human animals that comprise human immunoglobulin genes, such as XenoMouse®, and antibodies selected using in vitro methods, such as phage display, wherein the antibody repertoire is based on a human immunoglobulin sequences.

The terms “multivalent” or “polyvalent” antibody, as used herein, refer interchangeably to antibodies comprising more than one antigen binding domain, such as two (“bivalent”) or four (“tetravalent”) antigen binding domains. In some embodiments, the two or more antigen binding domains may be identical in amino acid sequence. In other embodiments, the antigen binding domains may differ in amino acid sequence. In some embodiments, a multivalent antibody comprises two or more sdAb variable regions, while in some embodiments, a multivalent antibody comprises two or more sets of heavy and light chain variable regions.

The term “leader sequence” refers to a sequence of amino acid residues located at the N terminus of a polypeptide that facilitates secretion of a polypeptide from a mammalian cell. A leader sequence may be cleaved upon export of the polypeptide from the mammalian cell, forming a mature protein. Leader sequences may be natural or synthetic, and they may be heterologous or homologous to the protein to which they are attached. Exemplary leader sequences include, but are not limited to, antibody leader sequences, such as, for example, the amino acid sequences of SEQ ID NOs: 3 and 4, which correspond to human light and heavy chain leader sequences, respectively. Nonlimiting exemplary leader sequences also include leader sequences from heterologous proteins. In some embodiments, an antibody lacks a leader sequence. In some embodiments, an antibody comprises at least one leader sequence, which may be selected from native antibody leader sequences and heterologous leader sequences.

The term “isolated” as used herein refers to a molecule that has been separated from at least some of the components with which it is typically found in nature. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, e.g., in the case of an RNA polynucleotide. Thus, a DNA polynucleotide that is contained in a vector inside a host cell may be referred to as “isolated” so long as that polynucleotide is not found in that vector in nature.

The term “elevated level” means a higher level of a protein in a particular tissue of a subject relative to the same tissue in a control, such as an individual or individuals who are not suffering from cancer or other condition described herein. The elevated level may be the result of any mechanism, such as increased expression, increased stability, decreased degradation, increased secretion, decreased clearance, etc., of the protein.

The term “reduce” or “reduces,” in the context of the level of a protein in a particular tissue, means to lower the level of a protein in a particular tissue of a subject by at least 10%.

The term “resistant,” when used in the context of resistance to a therapeutic agent, means a decreased response or lack of response to a standard dose of the therapeutic agent, relative to the subject's response to the standard dose of the therapeutic agent in the past, or relative to the expected response of a similar subject with a similar disorder to the standard dose of the therapeutic agent. Thus, in some embodiments, a subject may be resistant to a therapeutic agent although the subject has not previously been given the therapeutic agent, or the subject may develop resistance to the therapeutic agent after having responded to the agent on one or more previous occasions.

The terms “subject” and “patient” are used interchangeably herein to refer to a human. In some embodiments, methods of treating other mammals, including, but not limited to, rodents, simians, felines, canines, equines, bovines, porcines, ovines, caprines, mammalian laboratory animals, mammalian farm animals, mammalian sport animals, and mammalian pets, are also provided.

The term “sample,” as used herein, refers to a composition that is obtained or derived from a subject that contains a cellular and/or other molecular entity that is to be characterized, quantitated, and/or identified, for example based on physical, biochemical, chemical and/or physiological characteristics. An exemplary sample is a tissue sample.

The term “tissue sample” refers to a collection of similar cells obtained from a tissue of a subject. The source of the tissue sample may be solid tissue as from a fresh, frozen and/or preserved organ or tissue sample or biopsy or aspirate; blood or any blood constituents; bodily fluids such as cerebral spinal fluid, amniotic fluid, peritoneal fluid, synovial fluid, or interstitial fluid; cells from any time in gestation or development of the subject. The tissue sample may also be primary or cultured cells or cell lines. Optionally, the tissue sample is obtained from a disease tissue/organ, e.g. a tumor biopsy or synovial biopsy tissue sample. The tissue sample may contain compounds that are not naturally intermixed with the tissue in nature such as preservatives, anticoagulants, buffers, fixatives, nutrients, antibiotics, or the like. A “control sample” or “control tissue”, as used herein, refers to a sample, cell, or tissue obtained from a source known, or believed, not to be afflicted with the disease for which the subject is being treated.

For the purposes herein a “section” of a tissue sample means a part or piece of a tissue sample, such as a thin slice of tissue or cells cut from a solid tissue sample.

The term “cancer” is used herein to refer to a group of cells that exhibit abnormally high levels of proliferation and growth. A cancer may be benign (also referred to as a benign tumor), pre-malignant, or malignant. Cancer cells may be solid cancer cells or leukemic cancer cells. The term “cancer growth” is used herein to refer to proliferation or growth by a cell or cells that comprise a cancer that leads to a corresponding increase in the size or extent of the cancer.

Examples of cancer include but are not limited to, carcinoma, lymphoma, blastoma, sarcoma, and leukemia. More particular nonlimiting examples of such cancers include squamous cell cancer, small-cell lung cancer, pituitary cancer, esophageal cancer, astrocytoma, soft tissue sarcoma, non-small cell lung cancer (including squamous cell non-small cell lung cancer), adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, renal cell carcinoma, liver cancer, prostate cancer, vulval cancer, thyroid cancer, hepatic carcinoma, brain cancer, endometrial cancer, testis cancer, cholangiocarcinoma, gallbladder carcinoma, gastric cancer, melanoma, and various types of head and neck cancer (including squamous cell carcinoma of the head and neck).

The term “recurrent cancer” refers to a cancer that has returned after a previous treatment regimen, following which there was a period of time during which the cancer could not be detected, or during which tumors had shrunk, or during which disease was stable, or during which the cancer was considered to be in remission.

The term “progressive cancer” is a cancer that has increased in size or tumor spread since the beginning of a treatment regimen. In certain embodiments, a progressive cancer is a cancer that has increased in size or tumor spread by at least 10%, at least 20%, at least 30%, at least 40%, or at least 50% since the beginning of a treatment regimen.

The terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug. “Promoting cancer regression” means that administering an effective amount of the drug, alone or in combination with another anti-cancer agent, results in a reduction in tumor growth or size, necrosis of the tumor, a decrease in severity of at least one disease symptom, an increase in frequency and duration of disease symptom-free periods, or a prevention of impairment or disability due to the disease affliction.

By way of example for the treatment of tumors, a therapeutically effective amount of an anti-cancer agent may inhibit cell growth, inhibit tumor growth, or reduce tumor size by at least about 5%, at least about 10%, by at least about 15%, at least about 20%, by at least about 25%, by at least about 30%, by at least about 40%, by at least about 50%, by at least about 60%, by at least about 70%, or by at least about 80%, by at least about 90%, by at least about 95%, or by at least about 100% relative to untreated subjects, relative to baseline, or, in certain embodiments, relative to patients treated with a standard-of-care therapy.

“Treatment,” as used herein, refers to therapeutic treatment, for example, wherein the object is to slow down (lessen) the targeted pathologic condition or disorder as well as, for example, wherein the object is to inhibit recurrence of the condition or disorder. In certain embodiments, the term “treatment” covers any administration or application of a therapeutic for disease in a patient, and includes inhibiting or slowing the disease or progression of the disease; partially or fully relieving the disease, for example, by causing regression, or restoring or repairing a lost, missing, or defective function; stimulating an inefficient process; or causing the disease plateau to have reduced severity. The term “treatment” also includes reducing the severity of any phenotypic characteristic and/or reducing the incidence, degree, or likelihood of that characteristic. Those in need of treatment include those already with the disorder as well as those at risk of recurrence of the disorder or those in whom a recurrence of the disorder is to be prevented or slowed down.

Administration of a therapeutic agent “in combination with” one or more further therapeutic agents includes simultaneous (concurrent) and consecutive (sequential) administration in any order. For example, “concurrent” administration herein comprises administration of two or more agents on the same day, for example, during a single clinic, outpatient, or hospital visit. “Consecutive” or “sequential” administration herein means administration of two or more agents on different days.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed. For example, if the therapeutic agent is to be administered orally, the carrier may be a gel capsule. If the therapeutic agent is to be administered subcutaneously, the carrier ideally is not irritable to the skin and does not cause injection site reaction.

### Anti-GITR Antibodies

Anti-GITR antibodies herein bind to GITR and thereby activate GITR signaling function. Anti-GITR antibodies may bind to GITR and thereby activate GITR signaling function, for example, by activation of NF-κB response. This can be assayed using a system that monitors NF-κB-driven production of a reporter, secreted alkaline phosptatase (SEAP), as described in Example 5 of WO 2017/015623. As described therein, HEK293 cell lines containing an NF-κB-driven SEAP reporter gene (obtained from Invivogen, San Diego, Calif., USA) were stably transfected with GITR and the cell lines were then incubated with titrating doses of anti-GITR antibodies overnight at 37° C. SEAP reporter gene expression at each dose was then quantified in the cell culture supernatant by hydrolysis of a chromogenic substrate by monitoring changes of optical density at 650 nanometers. An increase in SEAP production in this assay over background due to addition of the antibody indicates that the antibody activates GITR signaling function. It is believed that the NF-⁻03 activation occurs due to trimerization of GITR by the bound antibody.

In some embodiments, an anti-GITR antibody herein may have one or more of the following properties: (a) comprises a GITR binding domain with a K_{D} for GITR of less than 10 nM; (b) binds to both human and cynomolgus monkey GITR; (c) blocks binding between GITR and its ligand GITRL; and (d) costimulate an anti-tumor response while also inhibiting the suppressive effect of T regulatory (Treg) cells.

An anti-GITR antibody, in some embodiments, may comprise at least one polypeptide that specifically binds GITR. In some embodiments, the polypeptide comprises at least one GITR-binding domain comprising three complementarity determining regions (CDRs) derived, for example, from a single domain antibody. In some embodiments, the at least one GITR-binding domain comprises a complementarity determining region 1 (CDR1) comprising the amino acid sequence of SEQ ID NO: 120, a complementarity determining region 2 (CDR2) comprising the amino acid sequence of SEQ ID NO: 121, and a complementarity determining region 3 (CDR3) comprising the amino acid sequence of SEQ ID NO: 122. In some embodiments, the antibodies are polyvalent (or multivalent), and comprise more than one such GITR-binding domain with the above set of CDRs.

In some embodiments, the anti-GITR antibody may comprise at least one polypeptide that comprises at least one GITR-binding domain, wherein the GITR-binding domain in turn comprises the amino acid sequence of SEQ ID NO: 119, or a sequence that is at least 90%, at least 91%, at least 92%, at least 93%, at least 94%, at least 95%, at least 96%, at least 97%, at least 98%, or at least 99% identical to SEQ ID NO: 119. In some embodiments, an anti-GITR antibody comprises two, three, or four GITR-binding domains comprising the amino acid sequence of SEQ ID NO: 119, or a sequence that is at least 90%, at least 91%, at least 92%, at least 93%, at least 94%, at least 95%, at least 96%, at least 97%, at least 98%, or at least 99% identical to SEQ ID NO: 119.

In some embodiments, an anti-GITR antibody comprises a multivalent fusion protein comprising two or more GITR-binding domains fused to a human constant region, such as a human IgG Fc. In some such embodiments, the two or more GITR-binding domains are in tandem. In some embodiments, the GITR-binding domains are derived from single domain antibodies and comprise three complementarity determining regions (CDRs). In some embodiments, at least one or all of the GITR-binding domains comprise a complementarity determining region 1 (CDR1) comprising the amino acid sequence of SEQ ID NO: 120, a complementarity determining region 2 (CDR2) comprising the amino acid sequence of SEQ ID NO: 121, and a complementarity determining region 3 (CDR3) comprising the amino acid sequence of SEQ ID NO: 122. In some such embodiments, the human IgG Fc is a human IgG1, IgG2, IgG3, or IgG4. In some embodiments, the multivalent fusion protein comprises two, three, or four GITR-binding domains in tandem, each with the above set of CDRs, fused to a human IgG Fc selected from a human IgG1, IgG2, IgG3, and IgG4 Fc.

In some embodiments, an anti-GITR antibody comprises a multivalent fusion protein comprising two or more GITR-binding domains fused to a human constant region, such as a human IgG Fc, e.g., a human IgG1, IgG2, IgG3, or IgG4 Fc. In some such embodiments, the two or more GITR-binding domains are in tandem. In some such embodiments, at least one or all of the GITR-binding domains comprise the amino acid sequence of SEQ ID NO: 119, or a sequence that is at least 90%, at least 91%, at least 92%, at least 93%, at least 94%, at least 95%, at least 96%, at least 97%, at least 98%, or at least 99% identical to SEQ ID NO: 119. In some such embodiments, the human constant region is a human IgG Fc, such as a human IgG1, IgG2, IgG3, or IgG4 Fc. In some embodiments, the multivalent fusion protein comprises two, three, or four GITR-binding domains in tandem, each comprising the amino acid sequence of SEQ ID NO:19, fused to a human IgG Fc selected from a human IgG1, IgG2, IgG3, and IgG4.

Tetravalent Molecules

In some embodiments, an anti-GITR antibody comprises a tetravalent molecule comprising two copies of a fusion protein having the structure: (GITR-BD)-Linker-(GITR-BD)-Linker-Hinge-Fc, wherein (a) GITR-BD is a GITR binding domain comprising (i) a complementarity determining region 1 (CDR1) comprising the amino acid sequence of SEQ ID NO: 120; a complementarity determining region 2 (CDR2) comprising the amino acid sequence of SEQ ID NO: 121; and a complementarity determining region 3 (CDR3) comprising the amino acid sequence of SEQ ID NO: 122; or comprising (ii) the amino acid sequence of SEQ ID NO: 119, or a sequence that is at least 90%, at least 91%, at least 92%, at least 93%, at least 94%, at least 95%, at least 96%, at least 97%, at least 98%, or at least 99% identical to SEQ ID NO: 119; (b) Linker is a linker polypeptide; (c) Hinge is a polypeptide derived from an immunoglobulin hinge region; and (d) Fc is an immunoglobulin Fc region polypeptide.

In some embodiments in which the fusion protein of a tetravalent molecule comprises a Hinge, the Hinge comprises the amino acid sequence of SEQ ID NO:7, 8, or 9. For example, the Hinge may comprise a modified IgG1 hinge comprising the amino acid sequence of EPKSSDKTHTCPPC (SEQ ID NO: 129), wherein the Cys220 that forms a disulfide bond with the C-terminal cysteine of the light chain is mutated to serine, e.g., Cys220Ser (C220S). In other embodiments, the fusion protein may comprise a Hinge comprising the amino acid sequence DKTHTCPPC (SEQ ID NO: 130). In some embodiments, the Hinge comprises a hinge from IgG4 that is modified, for example to prevent or reduce strand exchange, e.g., comprising the amino acid sequence ESKYGPPCPPC (SEQ ID NO: 131), in which Ser228 is mutated to Pro (S228P).

In some embodiments in which the fusion protein of a tetravalent molecule comprises a Linker, the Linker comprises an amino acid sequence selected from GG, GGG, and any one of SEQ ID NOs: 134 to 140. In some embodiments, the Linker comprises an amino acid sequence selected from SEQ ID NOs: 134 and 138. In some embodiments, a fusion protein of a tetravalent molecule has a Hinge comprising SEQ ID NO:130 and a Linker comprising SEQ ID NO:134 or 138.

In some embodiments in which the fusion protein of a tetravalent molecule comprises an Fc, the Fc is a human Fc, such as a human IgG1, IgG2, IgG3, or IgG4 Fc. In some embodiments, the Fc comprises an amino acid sequence selected from SEQ ID NOs: 123-128, or a sequence that is at least 90%, at least 91%, at least 92%, at least 93%, at least 94%, at least 95%, at least 96%, at least 97%, at least 98%, or at least 99% identical to one of SEQ ID NOs: 123-128. In some embodiments, the Fc comprises a human IgG1 amino acid sequence such as SEQ ID NO: 123.

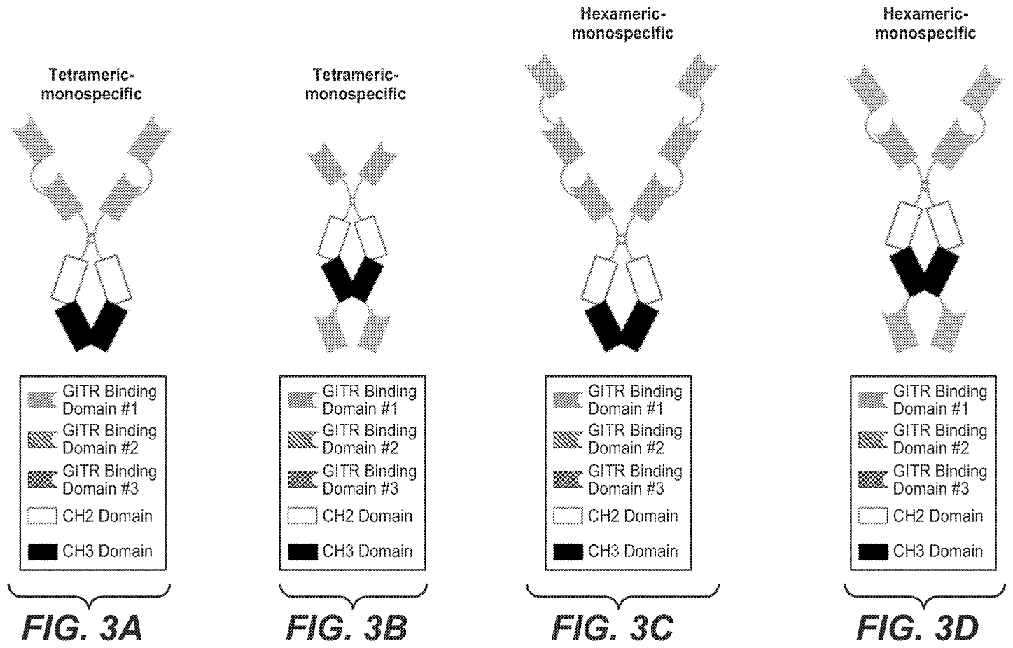

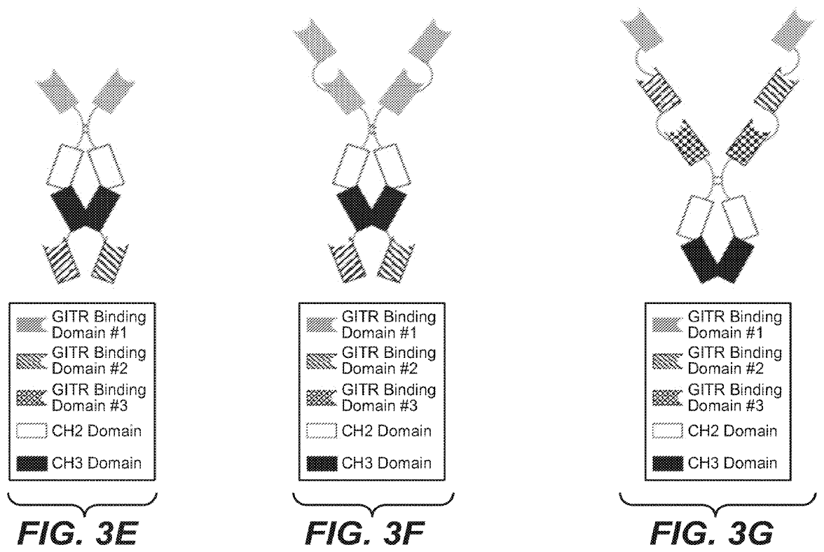

Exemplary tetravalent molecules are shown in FIGS. 3A and 3B. FIG. 3A, for instance, illustrates an exemplary tetravalent molecule with a (GITR-BD)-Linker-(GITR-BD)-Linker-Hinge-Fc architecture, wherein the Fc molecule comprises CH2 and CH3 domains. FIG. 3B, for instance, illustrates an alternative (GITR-BD)-Hinge-Fc-Linker-(GITR-BD) architecture. FIGS. 3C and 3D show hexavalent molecules having structures related to these two tetravalent molecules, i.e., (GITR-BD)-Linker-(GITR-BD)-Linker-(GITR-BD)-Linker-Hinge-Fc in FIG. 3C and (GITR-BD)-Linker-(GITR-BD)-Linker-Hinge-Fc-Linker-(GITR-BD) in FIG. 3D. FIGS. 3E-3G show exemplary tetravalent and hexavalent molecules having different GITR binding domains.

In some embodiments, an anti-GITR antibody is a tetravalent molecule comprising two copies of a polypeptide comprising the amino acid sequence of SEQ ID NO:118 or a sequence that is at least 90%, at least 91%, at least 92%, at least 93%, at least 94%, at least 95%, at least 96%, at least 97%, at least 98%, or at least 99% identical to SEQ ID NO:118.

Fc Regions

In any of the foregoing embodiments, an Fc may comprise an amino acid sequence selected from SEQ ID NOs: 123-128, or a sequence that is at least 90%, at least 91%, at least 92%, at least 93%, at least 94%, at least 95%, at least 96%, at least 97%, at least 98%, or at least 99% identical to one of SEQ ID NOs: 123-128. In some embodiments, the Fc comprises a human IgG1 amino acid sequence such as SEQ ID NO: 123. In some embodiments, the Fc comprises the amino acid sequence of SEQ ID NO: 123, but where position Asn297 (boxed in the sequence shown in the sequence table) is modified to inhibit fucosylation. In some embodiments, the Fc is a human IgG1 Fc, but where one or more of positions Leu235, Leu236, and Gly237 have been modified to other amino acids (boxed in the sequence shown in the sequence table). In some embodiments, the Fc comprises a human IgG1 Fc lacking Lys447. In some embodiments, the Fc is a human IgG1 Fc that lacks an amino acid at one or more of Glu233, Leu234, or Leu235, as provided, for example, in SEQ ID NO: 124. In some embodiments, the Fc comprises a human IgG2 Fc, e.g. SEQ ID NO: 125. In some embodiments, the Fc comprises a human IgG2 Fc that is modified, for example mutated at Asn297 (boxed in the sequence table) or that lacks Lys447. In some embodiments, the Fc comprises a human IgG3 Fc, e.g. SEQ ID NO: 126. In some embodiments, the Fc comprises a human IgG3 Fc that is modified, for example, mutated at Asn297, contains an Arg to His substitution at position 435 (both boxed in the sequence table), or that lacks Lys447. In some embodiments, the Fc comprises a human IgG4 Fc, e.g. SEQ ID NO: 127 or 128. In some embodiments, the Fc comprises a human IgG4 Fc that is modified, for example mutated at position Leu23 5 or Asn297 (both boxed in the sequence table), or that lacks Lys447.

In some embodiments, the human IgG Fc region is modified to enhance FcRn binding. Examples of Fc mutations that may enhance binding to FcRn are Met252Tyr, Ser254Thr, Thr256Glu (M252Y, S254T, T256E, respectively) (Kabat numbering, Dall'Acqua et al 2006, J. Biol Chem Vol. 281(33) 23514-23524), Met428Leu and Asn434Ser (M428L, N434S) (Zalevsky et al 2010 Nature Biotech, Vol. 28(2) 157-159), or Met252Ile, Thr256Asp, Met428Leu (M252I, T256D, M428L, respectively), (EU index of Kabat et al 1991 Sequences of Proteins of Immunological Interest).

In some embodiments, a mutated Fc may also include the following substitutions: Met252Tyr and Met428Leu (M252Y, M428L) using the Kabat numbering system.

In some embodiments, the human IgG Fc region may be modified to alter antibody-dependent cellular cytotoxicity (ADCC) and/or complement-dependent cytotoxicity (CDC), e.g., the amino acid modifications described in Natsume et al., 2008 Cancer Res, 68(10): 3863-72; Idusogie et al., 2001 J Immunol, 166(4): 2571-5; Moore et al., 2010 mAbs, 2(2): 181-189; Lazar et al., 2006 PNAS, 103(11): 4005-4010, Shields et al., 2001 JBC, 276(9): 6591-6604; Stavenhagen et al., 2007 Cancer Res, 67(18): 8882-8890; Stavenhagen et al., 2008 Advan. Enzyme Regul., 48: 152-164; Alegre et al, 1992 J Immunol, 148: 3461-3468; Reviewed in Kaneko and Niwa, 2011 Biodrugs, 25(1):1-11. Examples of mutations that may enhance ADCC include modification at Ser239 and Ile332, for example Ser239Asp and Ile332Glu (S239D, 1332E). Examples of mutations that may enhance CDC include modifications at Lys326 and Glu333. In some embodiments, the Fc region is modified at one or both of these positions, for example Lys326Ala and/or Glu333Ala (K326A and E333A) using the Kabat numbering system.

In some embodiments, the human IgG Fc region may be modified to induce heterodimerization. For example, having an amino acid modification within the CH3 domain at Thr366, which when replaced with a more bulky amino acid, e.g., Typ (T366W), is able to preferentially pair with a second CH3 domain having amino acid modifications to less bulky amino acids at positions Thr366, Leu368, and Tyr407, e.g., Ser, Ala and Val, respectively (T366S/L368A/Y407V). Further CH3 domain modifications, for example, can include changing Ser354 to Cys (S354C) and Y349 to Cys (Y349C) on opposite CH3 domains (Reviewed in Carter, 2001 Journal of Immunological Methods, 248: 7-15).

In some embodiments, the human IgG Fc region is modified to prevent or reduce dimerization of Fc domains. For example, residue Thr366 can be substituted with a charged residue, e.g. Thr366Lys, Thr366Arg, Thr366Asp, or Thr366Glu (T366K, T366R, T366D, or T366E, respectively), which may in some cases prevent CH3-CH3 dimerization.

In some embodiments, the Fc region may be altered at one or more of the following positions to reduce Fc receptor binding: Leu 234 (L234), Leu235 (L235), Asp265 (D265), Asp270 (D270), Ser298 (S298), Asn297 (N297), Asn325 (N325) or Ala327 (A327). For example, Leu 234Ala (L234A), Leu235Ala (L235A), Asp265Asn (D265N), Asp270Asn (D270N), Ser298Asn (S298N), Asn297Ala (N297A), Asn325Glu (N325E) or Ala327Ser (A327S). In some embodiments, modifications within the Fc region may reduce binding to Fc-receptor-gamma receptors while having minimal impact on binding to the neonatal Fc receptor (FcRn).

### Anti-CSF1R Antibodies

Anti-CSF1R antibodies include, but are not limited to, humanized antibodies, chimeric antibodies, mouse antibodies, human antibodies, and antibodies comprising the heavy chain and/or light chain CDRs discussed herein.

In some embodiments, exemplary anti-CSF1R antibodies include, for example, antibody species disclosed in WO2013/132044, WO2009/026303, WO2011/140249, and WO2009/112245. Exemplary anti-CSF1R antibodies include, for example, RG7155 (see WO2013/132044) and AMG-820 (see WO2009/026303). Thus, for example, in some embodiments, the anti-CSF1R antibody comprises the heavy chain and light chain CDRs of RG7155. In some embodiments, the anti-CSF1R antibody comprises the heavy chain and light chain variable regions of RG7155. In some embodiments, the anti-CSF1R antibody comprises the heavy and light chains of RG7155. In some embodiments, the anti-CSF1R antibody is RG7155. For example, in some embodiments, the anti-CSF1R antibody comprises the heavy chain and light chain CDRs of AMG-820. In some embodiments, the anti-CSF1R antibody comprises the heavy chain and light chain variable regions of AMG-820. In some embodiments, the anti-CSF1R antibody comprises the heavy and light chains of AMG-820. In some embodiments, the anti-CSF1R antibody is AMG-820.

Exemplary Humanized Antibodies

In some embodiments, humanized antibodies that bind CSF1R are provided. Humanized antibodies may be useful as therapeutic molecules because humanized antibodies may reduce or eliminate the human immune response to non-human antibodies (such as the human anti-mouse antibody (HAMA) response), which can result in an immune response to an antibody therapeutic, and decreased effectiveness of the therapeutic.

Nonlimiting exemplary humanized antibodies include huAb 1 through huAb16, described herein. Nonlimiting exemplary humanized antibodies also include antibodies comprising a heavy chain variable region of an antibody selected from huAb1 to huAb16 and/or a light chain variable region of an antibody selected from huAb1 to huAb16. Nonlimiting exemplary humanized antibodies include antibodies comprising a heavy chain variable region selected from SEQ ID NOs: 39 to 45 and/or a light chain variable region selected from SEQ ID NOs: 46 to 52. Exemplary humanized antibodies also include, but are not limited to, humanized antibodies comprising heavy chain CDR1, CDR2, and CDR3, and/or light chain CDR1, CDR2, and CDR3 of an antibody selected from 0301, 0302, and 0311.

In some embodiments, a humanized anti-CSF1R antibody comprises heavy chain CDR1, CDR2, and CDR3 and/or a light chain CDR1, CDR2, and CDR3 of an antibody selected from 0301, 0302, and 0311. Nonlimiting exemplary humanized anti-CSF1R antibodies include antibodies comprising sets of heavy chain CDR1, CDR2, and CDR3 selected from: SEQ ID NOs: 15, 16, and 17; SEQ ID NOs: 21, 22, and 23; and SEQ ID NOs: 27, 28, and 29. Nonlimiting exemplary humanized anti-CSF1R antibodies also include antibodies comprising sets of light chain CDR1, CDR2, and CDR3 selected from: SEQ ID NOs: 18, 19, and 20; SEQ ID NOs: 24, 25, and 26; and SEQ ID NOs: 30, 31, and 32.

Nonlimiting exemplary humanized anti-CSF1R antibodies include antibodies comprising the sets of heavy chain CDR1, CDR2, and CDR3, and light chain CDR1, CDR2, and CDR3 in Table 1 (SEQ ID NOs shown; see Table 8 for sequences). Each row of Table 1 shows the heavy chain CDR1, CDR2, and CDR3, and light chain CDR1, CDR2, and CDR3 of an exemplary antibody.

TABLE 1
Heavy chain and light chain CDRs

| | Heavy chain | | | Light chain | | |
|---|---|---|---|---|---|---|
| | CDR1 | CDR2 | CDR3 | CDR1 | CDR2 | CDR3 |
| Ab | SEQ ID | SEQ ID | SEQ ID | SEQ ID | SEQ ID | SEQ ID |
| 0301 | 15 | 16 | 17 | 18 | 19 | 20 |
| 0302 | 21 | 22 | 23 | 24 | 25 | 26 |
| 0311 | 27 | 28 | 29 | 30 | 31 | 32 |

Further Exemplary Humanized Antibodies

In some embodiments, a humanized anti-CSF1R antibody comprises a heavy chain comprising a variable region sequence that is at least 90%, at least 91%, at least 92%, at least 93%, at least 94%, at least 95%, at least 96%, at least 97%, at least 98%, or at least 99% identical to a sequence selected from SEQ ID NOs: 9, 11, 13, and 39 to 45, and wherein the antibody binds CSF1R. In some embodiments, a humanized anti-CSF1R antibody comprises a light chain comprising a variable region sequence that is at least 90%, at least 91%, at least 92%, at least 93%, at least 94%, at least 95%, at least 96%, at least 97%, at least 98%, or at least 99% identical to a sequence selected from SEQ ID NOs: 10, 12, 14, and 46 to 52, wherein the antibody binds CSF1R. In some embodiments, a humanized anti-CSF1R antibody comprises a heavy chain comprising a variable region sequence that is at least 90%, at least 91%, at least 92%, at least 93%, at least 94%, at least 95%, at least 96%, at least 97%, at least 98%, or at least 99% identical to a sequence selected from SEQ ID NOs: 9, 11, 13, and 39 to 45; and a light chain comprising a variable region sequence that is at least 90%, at least 91%, at least 92%, at least 93%, at least 94%, at least 95%, at least 96%, at least 97%, at least 98%, or at least 99% identical to a sequence selected from SEQ ID NOs: 10, 12, 14, and 46 to 52; wherein the antibody binds CSF1R.

In some embodiments, a humanized anti-CSF1R antibody comprises at least one of the CDRs discussed herein. That is, in some embodiments, a humanized anti-CSF1R antibody comprises at least one CDR selected from a heavy chain CDR1 discussed herein, a heavy chain CDR2 discussed herein, a heavy chain CDR3 discussed herein, a light chain CDR1 discussed herein, a light chain CDR2 discussed herein, and a light chain CDR3 discussed herein. Further, in some embodiments, a humanized anti-CSF1R antibody comprises at least one mutated CDR based on a CDR discussed herein, wherein the mutated CDR comprises 1, 2, 3, or 4 amino acid substitutions relative to the CDR discussed herein. In some embodiments, one or more of the amino acid substitutions are conservative amino acid substitutions. One skilled in the art can select one or more suitable conservative amino acid substitutions for a particular CDR sequence, wherein the suitable conservative amino acid substitutions are not predicted to significantly alter the binding properties of the antibody comprising the mutated CDR.

Exemplary humanized anti-CSF1R antibodies also include antibodies that compete for binding to CSF1R with an antibody described herein. Thus, in some embodiments, a humanized anti-CSF1R antibody is provided that competes for binding to CSF1R with an antibody selected from Fabs 0301, 0302, and 0311; and bivalent (i.e., having two heavy chains and two light chains) antibody versions of those Fabs.

Exemplary Humanized Antibody Constant Regions

In some embodiments, a humanized antibody described herein comprises one or more human constant regions. In some embodiments, the human heavy chain constant region is of an isotype selected from IgA, IgG, and IgD. In some embodiments, the human light chain constant region is of an isotype selected from κ and λ. In some embodiments, a humanized antibody described herein comprises a human IgG constant region. In some embodiments, a humanized antibody described herein comprises a human IgG4 heavy chain constant region. In some such embodiments, a humanized antibody described herein comprises an S241P mutation (Kabat numbering) in the human IgG4 constant region. In some embodiments, a humanized antibody described herein comprises a human IgG4 constant region and a human κ light chain.

The choice of heavy chain constant region can determine whether or not an antibody will have effector function in vivo. Such effector function, in some embodiments, includes antibody-dependent cell-mediated cytotoxicity (ADCC) and/or complement-dependent cytotoxicity (CDC), and can result in killing of the cell to which the antibody is bound. In some methods of treatment, including methods of treating some cancers, cell killing may be desirable, for example, when the antibody binds to a cell that supports the maintenance or growth of the tumor. Exemplary cells that may support the maintenance or growth of a tumor include, but are not limited to, tumor cells themselves, cells that aid in the recruitment of vasculature to the tumor, and cells that provide ligands, growth factors, or counter-receptors that support or promote tumor growth or tumor survival. In some embodiments, when effector function is desirable, an anti-CSF1R antibody comprising a human IgG1 heavy chain or a human IgG3 heavy chain is selected.

An anti-CSF1R antibody may be humanized by any method. Nonlimiting exemplary methods of humanization include methods described, e.g., in U.S. Pat. Nos. 5,530,101; 5,585,089; 5,693,761; 5,693,762; 6,180,370; Jones et al., Nature 321: 522-525 (1986); Riechmann et al., Nature 332: 323-27 (1988); Verhoeyen et al., Science 239: 1534-36 (1988); and U.S. Publication No. US 2009/0136500.

As noted above, a humanized antibody is an antibody in which at least one amino acid in a framework region of a non-human variable region has been replaced with the amino acid from the corresponding location in a human framework region. In some embodiments, at least two, at least three, at least four, at least five, at least six, at least seven, at least eight, at least nine, at least 10, at least 11, at least 12, at least 15, or at least 20 amino acids in the framework regions of a non-human variable region are replaced with an amino acid from one or more corresponding locations in one or more human framework regions.

In some embodiments, some of the corresponding human amino acids used for substitution are from the framework regions of different human immunoglobulin genes. That is, in some such embodiments, one or more of the non-human amino acids may be replaced with corresponding amino acids from a human framework region of a first human antibody or encoded by a first human immunoglobulin gene, one or more of the non-human amino acids may be replaced with corresponding amino acids from a human framework region of a second human antibody or encoded by a second human immunoglobulin gene, one or more of the non-human amino acids may be replaced with corresponding amino acids from a human framework region of a third human antibody or encoded by a third human immunoglobulin gene, etc. Further, in some embodiments, all of the corresponding human amino acids being used for substitution in a single framework region, for example, FR2, need not be from the same human framework. In some embodiments, however, all of the corresponding human amino acids being used for substitution are from the same human antibody or encoded by the same human immunoglobulin gene.

In some embodiments, an anti-CSF1R antibody is humanized by replacing one or more entire framework regions with corresponding human framework regions. In some embodiments, a human framework region is selected that has the highest level of homology to the non-human framework region being replaced. In some embodiments, such a humanized antibody is a CDR-grafted antibody.

In some embodiments, following CDR-grafting, one or more framework amino acids are changed back to the corresponding amino acid in a mouse framework region. Such “back mutations” are made, in some embodiments, to retain one or more mouse framework amino acids that appear to contribute to the structure of one or more of the CDRs and/or that may be involved in antigen contacts and/or appear to be involved in the overall structural integrity of the antibody. In some embodiments, ten or fewer, nine or fewer, eight or fewer, seven or fewer, six or fewer, five or fewer, four or fewer, three or fewer, two or fewer, one, or zero back mutations are made to the framework regions of an antibody following CDR grafting.

In some embodiments, a humanized anti-CSF1R antibody also comprises a human heavy chain constant region and/or a human light chain constant region.

Exemplary Chimeric Anti-CSF1R Antibodies

In some embodiments, an anti-CSF1R antibody is a chimeric antibody. In some embodiments, an anti-CSF1R antibody comprises at least one non-human variable region and at least one human constant region. In some such embodiments, all of the variable regions of an anti-CSF1R antibody are non-human variable regions, and all of the constant regions of an anti-CSF1R antibody are human constant regions. In some embodiments, one or more variable regions of a chimeric antibody are mouse variable regions. The human constant region of a chimeric antibody need not be of the same isotype as the non-human constant region, if any, it replaces. Chimeric antibodies are discussed, e.g., in U.S. Pat. No. 4,816,567; and Morrison et al. Proc. Natl. Acad. Sci. USA 81: 6851-55 (1984).

Nonlimiting exemplary chimeric antibodies include chimeric antibodies comprising the heavy and/or light chain variable regions of an antibody selected from 0301, 0302, and 0311. Additional nonlimiting exemplary chimeric antibodies include chimeric antibodies comprising heavy chain CDR1, CDR2, and CDR3, and/or light chain CDR1, CDR2, and CDR3 of an antibody selected from 0301, 0302, and 0311.

Nonlimiting exemplary chimeric anti-CSF1R antibodies include antibodies comprising the following pairs of heavy and light chain variable regions: SEQ ID NOs: 9 and 10; SEQ ID NOs: 11 and 12; and SEQ ID NOs: 13 and 14.

Nonlimiting exemplary anti-CSF1R antibodies include antibodies comprising a set of heavy chain CDR1, CDR2, and CDR3, and light chain CDR1, CDR2, and CDR3 shown above in Table 1.

Further Exemplary Chimeric Anti-CSF1R Antibodies

In some embodiments, a chimeric anti-CSF1R antibody comprises a heavy chain comprising a variable region sequence that is at least 90%, at least 91%, at least 92%, at least 93%, at least 94%, at least 95%, at least 96%, at least 97%, at least 98%, or at least 99% identical to a sequence selected from SEQ ID NOs: 9, 11, 13, and 39 to 45, wherein the antibody binds CSF1R. In some embodiments, a chimeric anti-CSF1R antibody comprises a light chain comprising a variable region sequence that is at least 90%, at least 91%, at least 92%, at least 93%, at least 94%, at least 95%, at least 96%, at least 97%, at least 98%, or at least 99% identical to a sequence selected from SEQ ID NOs: 10, 12, 14, and 46 to 52, wherein the antibody binds CSF1R. In some embodiments, a chimeric anti-CSF1R antibody comprises a heavy chain comprising a variable region sequence that is at least 90%, at least 91%, at least 92%, at least 93%, at least 94%, at least 95%, at least 96%, at least 97%, at least 98%, or at least 99% identical to a sequence selected from SEQ ID NOs: 9, 11, 13, and 39 to 45; and a light chain comprising a variable region sequence that is at least 90%, at least 91%, at least 92%, at least 93%, at least 94%, at least 95%, at least 96%, at least 97%, at least 98%, or at least 99% identical to a sequence selected from SEQ ID NOs: 10, 12, 14, and 46 to 52; wherein the antibody binds CSF1R.

In some embodiments, a chimeric anti-CSF1R antibody comprises at least one of the CDRs discussed herein. That is, in some embodiments, a chimeric anti-CSF1R antibody comprises at least one CDR selected from a heavy chain CDR1 discussed herein, a heavy chain CDR2 discussed herein, a heavy chain CDR3 discussed herein, a light chain CDR1 discussed herein, a light chain CDR2 discussed herein, and a light chain CDR3 discussed herein. Further, in some embodiments, a chimeric anti-CSF1R antibody comprises at least one mutated CDR based on a CDR discussed herein, wherein the mutated CDR comprises 1, 2, 3, or 4 amino acid substitutions relative to the CDR discussed herein. In some embodiments, one or more of the amino acid substitutions are conservative amino acid substitutions. One skilled in the art can select one or more suitable conservative amino acid substitutions for a particular CDR sequence, wherein the suitable conservative amino acid substitutions are not predicted to significantly alter the binding properties of the antibody comprising the mutated CDR.

Exemplary chimeric anti-CSF1R antibodies also include chimeric antibodies that compete for binding to CSF1R with an antibody described herein. Thus, in some embodiments, a chimeric anti-CSF1R antibody is provided that competes for binding to CSF1R with an antibody selected from Fabs 0301, 0302, and 0311; and bivalent (i.e., having two heavy chains and two light chains) antibody versions of those Fabs.

Exemplary Anti-CSF1R Chimeric Antibody Constant Regions

In some embodiments, a chimeric antibody described herein comprises one or more human constant regions. In some embodiments, the human heavy chain constant region is of an isotype selected from IgA, IgG, and IgD. In some embodiments, the human light chain constant region is of an isotype selected from κ and λ. In some embodiments, a chimeric antibody described herein comprises a human IgG constant region, such as an IgG1, IgG2, IgG3, or IgG4 constant region. In some embodiments, a chimeric antibody described herein comprises a human IgG4 heavy chain constant region. In some such embodiments, a chimeric antibody described herein comprises a human IgG4 constant region with an S241P mutation. In some embodiments, a chimeric antibody described herein comprises a human IgG4 constant region and a human κ light chain.

As noted above, whether or not effector function is desirable may depend on the particular method of treatment intended for an antibody. Thus, in some embodiments, when effector function is desirable, a chimeric anti-CSF1R antibody comprising a human IgG1 heavy chain constant region or a human IgG3 heavy chain constant region is selected. In some embodiments, when effector function is not desirable, a chimeric anti-CSF1R antibody comprising a human IgG4 or IgG2 heavy chain constant region is selected.

Exemplary Anti-CSF1R Human Antibodies

Human antibodies can be made by any suitable method. Nonlimiting exemplary methods include making human antibodies in transgenic mice that comprise human immunoglobulin loci. See, e.g., Jakobovits et al., Proc. Natl. Acad. Sci. USA 90: 2551-55 (1993); Jakobovits et al., Nature 362: 255-8 (1993); Lonberg et al., Nature 368: 856-9 (1994); and U.S. Pat. Nos. 5,545,807; 6,713,610; 6,673,986; 6,162,963; 5,545,807; 6,300,129; 6,255,458; 5,877,397; 5,874,299; and 5,545,806.

Nonlimiting exemplary methods also include making human antibodies using phage display libraries. See, e.g., Hoogenboom et al., J. Mol. Biol. 227: 381-8 (1992); Marks et al., J. Mol. Biol. 222: 581-97 (1991); and PCT Publication No. WO 99/10494.

In some embodiments, a human anti-CSF1R antibody binds to a polypeptide having the sequence of SEQ ID NO: 1. Exemplary human anti-CSF1R antibodies also include antibodies that compete for binding to CSF1R with an antibody described herein. Thus, in some embodiments, a human anti-CSF1R antibody is provided that competes for binding to CSF1R with an antibody selected from Fabs 0301, 0302, and 0311, and bivalent (i.e., having two heavy chains and two light chains) antibody versions of those Fabs.

In some embodiments, a human anti-CSF1R antibody comprises one or more human constant regions. In some embodiments, the human heavy chain constant region is of an isotype selected from IgA, IgG, and IgD. In some embodiments, the human light chain constant region is of an isotype selected from κ and λ. In some embodiments, a human antibody described herein comprises a human IgG constant region, such as an IgG1, IgG2, IgG3, or IgG4 constant region. In some embodiments, a human antibody described herein comprises a human IgG4 heavy chain constant region. In some such embodiments, a human antibody described herein comprises a human IgG4 heavy chain constant region with an S241P mutation. In some embodiments, a human antibody described herein comprises a human IgG4 constant region and a human κ light chain.

In some embodiments, when effector function is desirable, a human anti-CSF1R antibody comprising a human IgG1 heavy chain constant region or a human IgG3 heavy chain constant region is selected. In some embodiments, when effector function is not desirable, a human anti-CSF1R antibody comprising a human IgG4 or IgG2 heavy chain constant region is selected.

Additional Exemplary Anti-CSF1R Antibodies

Exemplary anti-CSF1R antibodies also include, but are not limited to, mouse, humanized, human, chimeric, and engineered antibodies that comprise, for example, one or more of the CDR sequences described herein. In some embodiments, an anti-CSF1R antibody comprises a heavy chain variable region described herein. In some embodiments, an anti-CSF1R antibody comprises a light chain variable region described herein. In some embodiments, an anti-CSF1R antibody comprises a heavy chain variable region described herein and a light chain variable region described herein. In some embodiments, an anti-CSF1R antibody comprises heavy chain CDR1, CDR2, and CDR3 described herein. In some embodiments, an anti-CSF1R antibody comprises light chain CDR1, CDR2, and CDR3 described herein. In some embodiments, an anti-CSF1R antibody comprises heavy chain CDR1, CDR2, and CDR3 described herein and light chain CDR1, CDR2, and CDR3 described herein.

In some embodiments, an anti-CSF1R antibody comprises a heavy chain variable region of an antibody selected from Fabs 0301, 0302, and 0311. Nonlimiting exemplary anti-CSF1R antibodies also include antibodies comprising a heavy chain variable region of an antibody selected from humanized antibodies huAb1 to huAb16. Nonlimiting exemplary anti-CSF1R antibodies include antibodies comprising a heavy chain variable region comprising a sequence selected from SEQ ID NOs: 9, 11, 13, and 39 to 45.

In some embodiments, an anti-CSF1R antibody comprises a light chain variable region of an antibody selected from Fabs 0301, 0302, and 0311. Nonlimiting exemplary anti-CSF1R antibodies also include antibodies comprising a light chain variable region of an antibody selected from humanized antibodies huAb1 to huAb16. Nonlimiting exemplary anti-CSF1R antibodies include antibodies comprising a light chain variable region comprising a sequence selected from SEQ ID NOs: 10, 12, 14, and 46 to 52.

In some embodiments, an anti-CSF1R antibody comprises a heavy chain variable region and a light chain variable region of an antibody selected from Fabs 0301, 0302, and 0311. Nonlimiting exemplary anti-CSF1R antibodies also include antibodies comprising a heavy chain variable region and a light chain variable region of an antibody selected from humanized antibodies huAb1 to huAb16. Nonlimiting exemplary anti-CSF1R antibodies include antibodies comprising the following pairs of heavy and light chain variable regions: SEQ ID NOs: 9 and 10; SEQ ID NOs: 11 and 12; and SEQ ID NOs: 13 and 14; SEQ ID NOs: 39 and 40; SEQ ID NOs: 41 and 42; SEQ ID NOs: 43 and 44; SEQ ID NOs: 45 and 46; SEQ ID NOs: 47 and 48; SEQ ID NOs: 49 and 50; and SEQ ID NOs: 51 and 52. Nonlimiting exemplary anti-CSF1R antibodies also include antibodies comprising the following pairs of heavy and light chains: SEQ ID NOs: 33 and 34; SEQ ID NOs: 35 and 36; and SEQ ID NOs: 37 and 38.

In some embodiments, an anti-CSF1R antibody comprises heavy chain CDR1, CDR2, and CDR3 of an antibody selected from Fabs 0301, 0302, and 0311. Nonlimiting exemplary anti-CSF1R antibodies include antibodies comprising sets of heavy chain CDR1, CDR2, and CDR3 selected from: SEQ ID NOs: 15, 16, and 17; SEQ ID NOs: 21, 22, and 23; and SEQ ID NOs: 27, 28,and 29.

In some embodiments, an anti-CSF1R antibody comprises light chain CDR1, CDR2, and CDR3 of an antibody selected from Fabs 0301, 0302, and 0311. Nonlimiting exemplary anti-CSF1R antibodies include antibodies comprising sets of light chain CDR1, CDR2, and CDR3 selected from: SEQ ID NOs: 18, 19, and 20; SEQ ID NOs: 24, 25, and 26; and SEQ ID NOs: 30, 31, and 32.

In some embodiments, an anti-CSF1R antibody comprises heavy chain CDR1, CDR2, and CDR3, and light chain CDR1, CDR2, and CDR3 of an antibody selected from Fabs 0301, 0302, and 0311.

Nonlimiting exemplary anti-CSF1R antibodies include antibodies comprising the sets of heavy chain CDR1, CDR2, and CDR3, and light chain CDR1, CDR2, and CDR3 shown above in Table 1.

Further Exemplary Anti-CSF1R Antibodies

In some embodiments, an anti-CSF1R antibody comprises a heavy chain comprising a variable region sequence that is at least 90%, at least 91%, at least 92%, at least 93%, at least 94%, at least 95%, at least 96%, at least 97%, at least 98%, or at least 99% identical to a sequence selected from SEQ ID NOs: 9, 11, 13, and 39 to 45, wherein the antibody binds CSF1R. In some embodiments, an anti-CSF1R antibody comprises a light chain comprising a variable region sequence that is at least 90%, at least 91%, at least 92%, at least 93%, at least 94%, at least 95%, at least 96%, at least 97%, at least 98%, or at least 99% identical to a sequence selected from SEQ ID NOs: 10, 12, 14, and 46 to 52, wherein the antibody binds CSF1R. In some embodiments, an anti-CSF1R antibody comprises a heavy chain comprising a variable region sequence that is at least 90%, at least 91%, at least 92%, at least 93%, at least 94%, at least 95%, at least 96%, at least 97%, at least 98%, or at least 99% identical to a sequence selected from SEQ ID NOs: 9, 11, 13, and 39 to 45; and a light chain comprising a variable region sequence that is at least 90%, at least 91%, at least 92%, at least 93%, at least 94%, at least 95%, at least 96%, at least 97%, at least 98%, or at least 99% identical to a sequence selected from SEQ ID NOs: 10, 12, 14, and 46 to 52; wherein the antibody binds CSF1R.

In some embodiments, an anti-CSF1R antibody comprises at least one of the CDRs discussed herein. That is, in some embodiments, an anti-CSF1R antibody comprises at least one CDR selected from a heavy chain CDR1 discussed herein, a heavy chain CDR2 discussed herein, a heavy chain CDR3 discussed herein, a light chain CDR1 discussed herein, a light chain CDR2 discussed herein, and a light chain CDR3 discussed herein. Further, in some embodiments, an anti-CSF1R antibody comprises at least one mutated CDR based on a CDR discussed herein, wherein the mutated CDR comprises 1, 2, 3, or 4 amino acid substitutions relative to the CDR discussed herein. In some embodiments, one or more of the amino acid substitutions are conservative amino acid substitutions. One skilled in the art can select one or more suitable conservative amino acid substitutions for a particular CDR sequence, wherein the suitable conservative amino acid substitutions are not predicted to significantly alter the binding properties of the antibody comprising the mutated CDR.

Exemplary anti-CSF1R antibodies also include antibodies that compete for binding to CSF1R with an antibody described herein. Thus, in some embodiments, an anti-CSF1R antibody is provided that competes for binding to CSF1R with an antibody selected from Fabs 0301, 0302, and 0311, and bivalent (i.e., having two heavy chains and two light chains) antibody versions of those Fabs.

Exemplary Anti-CSF1R Antibody Constant Regions

In some embodiments, an antibody described herein comprises one or more human constant regions. In some embodiments, the human heavy chain constant region is of an isotype selected from IgA, IgG, and IgD. In some embodiments, the human light chain constant region is of an isotype selected from κ and λ. In some embodiments, an antibody described herein comprises a human IgG constant region, such as an IgG1, IgG2, IgG3, or IgG4 constant region. In some embodiments, an antibody described herein comprises a human IgG4 heavy chain constant region. In some such embodiments, an antibody described herein comprises a human IgG4 heavy chain constant region with an S241P mutation. In some embodiments, an antibody described herein comprises a human IgG4 constant region and a human κ light chain.

As noted above, whether or not effector function is desirable may depend on the particular method of treatment intended for an antibody. Thus, in some embodiments, when effector function is desirable, an anti-CSF1R antibody comprising a human IgG1 heavy chain constant region or a human IgG3 heavy chain constant region is selected. In some embodiments, when effector function is not desirable, an anti-CSF1R antibody comprising a human IgG4 or IgG2 heavy chain constant region is selected.

Exemplary Anti-CSF1R Heavy Chain Variable Regions

In some embodiments, anti-CSF1R antibody heavy chain variable regions are provided. In some embodiments, an anti-CSF1R antibody heavy chain variable region is a mouse variable region, a human variable region, or a humanized variable region.

An anti-CSF1R antibody heavy chain variable region comprises a heavy chain CDR1, FR2, CDR2, FR3, and CDR3. In some embodiments, an anti-CSF1R antibody heavy chain variable region further comprises a heavy chain FR1 and/or FR4. Nonlimiting exemplary heavy chain variable regions include, but are not limited to, heavy chain variable regions having an amino acid sequence selected from SEQ ID NOs: 9, 11, 13, and 39 to 45.

In some embodiments, an anti-CSF1R antibody heavy chain variable region comprises a CDR1 comprising a sequence selected from SEQ ID NOs: 15, 21, and 27.

In some embodiments, an anti-CSF1R antibody heavy chain variable region comprises a CDR2 comprising a sequence selected from SEQ ID NOs: 16, 22, and 28.

In some embodiments, an anti-CSF1R antibody heavy chain variable region comprises a CDR3 comprising a sequence selected from SEQ ID NOs: 17, 23, and 29.

Nonlimiting exemplary heavy chain variable regions include, but are not limited to, heavy chain variable regions comprising sets of CDR1, CDR2, and CDR3 selected from: SEQ ID NOs: 15, 16, and 17; SEQ ID NOs: 21, 22, and 23; and SEQ ID NOs: 27, 28, and 29.

In some embodiments, an anti-CSF1R antibody heavy chain comprises a variable region sequence that is at least 90%, at least 91%, at least 92%, at least 93%, at least 94%, at least 95%, at least 96%, at least 97%, at least 98%, or at least 99% identical to a sequence selected from SEQ ID NOs: 9, 11, 13, and 39 to 45, wherein the heavy chain, together with a light chain, is capable of forming an antibody that binds CSF1R.

In some embodiments, an anti-CSF1R antibody heavy chain comprises at least one of the CDRs discussed herein. That is, in some embodiments, an anti-CSF1R antibody heavy chain comprises at least one CDR selected from a heavy chain CDR1 discussed herein, a heavy chain CDR2 discussed herein, and a heavy chain CDR3 discussed herein. Further, in some embodiments, an anti-CSF1R antibody heavy chain comprises at least one mutated CDR based on a CDR discussed herein, wherein the mutated CDR comprises 1, 2, 3, or 4 amino acid substitutions relative to the CDR discussed herein. In some embodiments, one or more of the amino acid substitutions are conservative amino acid substitutions. One skilled in the art can select one or more suitable conservative amino acid substitutions for a particular CDR sequence, wherein the suitable conservative amino acid substitutions are not predicted to significantly alter the binding properties of the heavy chain comprising the mutated CDR.

In some embodiments, a heavy chain comprises a heavy chain constant region. In some embodiments, a heavy chain comprises a human heavy chain constant region. In some embodiments, the human heavy chain constant region is of an isotype selected from IgA, IgG, and IgD. In some embodiments, the human heavy chain constant region is an IgG constant region. In some embodiments, a heavy chain comprises a human igG4 heavy chain constant region. In some such embodiments, the human IgG4 heavy chain constant region comprises an S241P mutation.

In some embodiments, when effector function is desirable, a heavy chain comprises a human IgG1 or IgG3 heavy chain constant region. In some embodiments, when effector function is less desirable, a heavy chain comprises a human IgG4 or IgG2 heavy chain constant region.

Exemplary Anti-CSF1R Light Chain Variable Regions

In some embodiments, anti-CSF1R antibody light chain variable regions are provided. In some embodiments, an anti-CSF1R antibody light chain variable region is a mouse variable region, a human variable region, or a humanized variable region.

An anti-CSF1R antibody light chain variable region comprises a light chain CDR1, FR2, CDR2, FR3, and CDR3. In some embodiments, an anti-CSF1R antibody light chain variable region further comprises a light chain FR1 and/or FR4. Nonlimiting exemplary light chain variable regions include light chain variable regions having an amino acid sequence selected from SEQ ID NOs: 10, 12, 14, and 46 to 52.

In some embodiments, an anti-CSF1R antibody light chain variable region comprises a CDR1 comprising a sequence selected from SEQ ID NOs: 18, 24 and 30.

In some embodiments, an anti-CSF1R antibody light chain variable region comprises a CDR2 comprising a sequence selected from SEQ ID NOs: 19, 25, and 31.

In some embodiments, an anti-CSF1R antibody light chain variable region comprises a CDR3 comprising a sequence selected from SEQ ID NOs: 20, 26, and 32.

Nonlimiting exemplary light chain variable regions include, but are not limited to, light chain variable regions comprising sets of CDR1, CDR2, and CDR3 selected from: SEQ ID NOs: 18, 19, and 20; SEQ ID NOs: 24, 25, and 26; and SEQ ID NOs: 30, 31, and 32.

In some embodiments, an anti-CSF1R antibody light chain comprises a variable region sequence that is at least 90%, at least 91%, at least 92%, at least 93%, at least 94%, at least 95%, at least 96%, at least 97%, at least 98%, or at least 99% identical to a sequence selected from SEQ ID NOs: 10, 12, 14, and 46 to 52, wherein the light chain, together with a heavy chain, is capable of forming an antibody that binds CSF1R.

In some embodiments, an anti-CSF1R antibody light chain comprises at least one of the CDRs discussed herein. That is, in some embodiments, an anti-CSF1R antibody light chain comprises at least one CDR selected from a light chain CDR1 discussed herein, a light chain CDR2 discussed herein, and a light chain CDR3 discussed herein. Further, in some embodiments, an anti-CSF1R antibody light chain comprises at least one mutated CDR based on a CDR discussed herein, wherein the mutated CDR comprises 1, 2, 3, or 4 amino acid substitutions relative to the CDR discussed herein. In some embodiments, one or more of the amino acid substitutions are conservative amino acid substitutions. One skilled in the art can select one or more suitable conservative amino acid substitutions for a particular CDR sequence, wherein the suitable conservative amino acid substitutions are not predicted to significantly alter the binding properties of the light chain comprising the mutated CDR.

In some embodiments, a light chain comprises a human light chain constant region. In some embodiments, a human light chain constant region is selected from a human κ and a human X, light chain constant region.

Exemplary Additional CSF1R Antibodies and Binding Molecules

In some embodiments, other anti-CSF1R antibodies are used. In some embodiments, exemplary anti-CSF1R antibodies include, for example, antibody species disclosed in WO2013/132044, WO2009/026303, WO2011/140249, and WO2009/112245. Exemplary anti-CSF1R antibodies include, for example, RG7155 (see WO2013/132044) and AMG-820 (see WO2009/026303). Thus, for example, in some embodiments, the anti-CSF1R antibody comprises the heavy chain and light chain CDRs of RG7155. In some embodiments, the anti-CSF1R antibody comprises the heavy chain and light chain variable regions of RG7155. In some embodiments, the anti-CSF1R antibody comprises the heavy and light chains of RG7155. In some embodiments, the anti-CSF1R antibody is RG7155. For example, in some embodiments, the anti-CSF1R antibody comprises the heavy chain and light chain CDRs of AMG-820. In some embodiments, the anti-CSF1R antibody comprises the heavy chain and light chain variable regions of AMG-820. In some embodiments, the anti-CSF1R antibody comprises the heavy and light chains of AMG-820. In some embodiments, the anti-CSF1R antibody is AMG-820.

Other types of antibody molecules include, but are not limited to, molecules comprising non-canonical scaffolds, such as anti-calins, adnectins, ankyrin repeats, etc. See, e.g., Hosse et al., Prot. Sci. 15:14 (2006); Fiedler, M. and Skerra, A., “Non-Antibody Scaffolds,” pp. 467-499 in Handbook of Therapeutic Antibodies, Dubel, S., ed., Wiley-VCH, Weinheim, Germany, 2007.

Exemplary Properties of anti-CSF1R antibodies

In some embodiments, an antibody having a structure described above binds to the CSF1R with a binding affinity (K_{D}) of less than 1 nM, blocks binding of CSF1 and/or IL-34 to CSF1R, and inhibits CSF1R phosphorylation induced by CSF1 and/or IL-34.

In some embodiments, an anti-CSF1R antibody binds to the extracellular domain of CSF1R (CSF1R-ECD). In some embodiments, an anti-CSF1R antibody has a binding affinity (K_{D}) for CSF1R of less than 1 nM, less than 0.5 nM, less than 0.1 nM, or less than 0.05 nM. In some embodiments, an anti-CSF1R antibody has a K_{D} of between 0.01 and 1 nM, between 0.01 and 0.5 nM, between 0.01 and 0.1 nM, between 0.01 and 0.05 nM, or between 0.02 and 0.05 nM.

In some embodiments, an anti-CSF1R antibody blocks binding of both CSF1 and IL-34 to CSF1R. In some embodiments, an anti-CSF1R antibody blocks ligand binding to CSF1R when it reduces the amount of detectable binding of a ligand to CSF1R by at least 50%, using the assay described, e.g., U.S. Pat. No. 8,206,715 B2, Example 7, which is incorporated herein by reference for any purpose. In some embodiments, an anti-CSF1R antibody reduces the amount of detectable binding of a ligand to CSF1R by at least 60%, at least 70%, at least 80%, or at least 90%. In some such embodiments, the anti-CSF1R antibody is said to block ligand binding by at least 50%, at least 60%, at least 70%, etc.

In some embodiments, an anti-CSF1R antibody inhibits ligand-induced CSF1R phosphorylation. In some embodiments, an anti-CSF1R antibody inhibits CSF1-induced CSF1R phosphorylation. In some embodiments, an anti-CSF1R antibody inhibits IL-34-induced CSF1R phosphorylation. In some embodiments, an anti-CSF1R antibody inhibits both CSF1-induced and IL-34-induced CSF1R phosphorylation. In some embodiments, an antibody is considered to “inhibit ligand-induced CSF1R phosphorylation” when it reduces the amount of detectable ligand-induced CSF1R phosphorylation by at least 50%, using the assay described, e.g., U.S. Pat. No. 8,206,715 B2, Example 6, which is incorporated herein by reference for any purpose. In some embodiments, an antibody reduces the amount of detectable ligand-induced CSF1R phosphorylation by at least 60%, at least 70%, at least 80%, or at least 90%. In some such embodiments, the antibody is said to inhibit ligand-induced CSF1R phosphorylation by at least at least 50%, at least 60%, at least 70%, etc.

In some embodiments, an antibody inhibits monocyte proliferation and/or survival responses in the presence of CSF1 and/or IL-34. In some embodiments, an antibody is considered to “inhibit monocyte proliferation and/or survival responses” when it reduces the amount of monocyte proliferation and/or survival responses in the presence of CSF1 and/or IL-34 by at least 50%, using the assay described, e.g., U.S. Pat. No. 8,206,715 B2, Example 10, which is incorporated herein by reference for any purpose. In some embodiments, an antibody reduces the amount of monocyte proliferation and/or survival responses in the presence of CSF1 and/or IL-34 by at least 60%, at least 70%, at least 80%, or at least 90%. In some such embodiments, the antibody is said to inhibit monocyte proliferation and/or survival responses by at least at least 50%, at least 60%, at least 70%, etc.

### Exemplary Anti-PD-1 Antibodies

PD-1 is a key immune checkpoint receptor expressed by activated T and B cells and mediates immunosuppression. PD-1 is a member of the CD28 family of receptors, which includes CD28, CTLA-4, ICOS, PD-1, and BTLA. Two cell surface glycoprotein ligands for PD-1 have been identified, Programmed Death Ligand-1 (PD-L1) and Programmed Death Ligand-2 (PD-L2), that are expressed on antigen-presenting cells as well as many human cancers and have been shown to down regulate T cell activation and cytokine secretion upon binding to PD-1. Inhibition of the PD-1/PD-L1 interaction mediates potent antitumor activity in preclinical models.

Human monoclonal antibodies (HuMAbs) that bind specifically to PD-1 with high affinity have been disclosed in U.S. Pat. No. 8,008,449. Other anti-PD-1 monoclonal antibodies (mAbs) have been described in, for example, U.S. Pat. Nos. 6,808,710, 7,488,802, 8,168,757 and 8,354,509, and PCT Publication No. WO 2012/145493. Each of the anti-PD-1 HuMAbs disclosed in U.S. Pat. No. 8,008,449 has been demonstrated to exhibit one or more of the following characteristics: (a) binds to human PD-1 with a K_{D} of 1×10⁻⁷ M or less, as determined by surface plasmon resonance using a Biacore biosensor system; (b) does not substantially bind to human CD28, CTLA-4 or ICOS; (c) increases T-cell proliferation in a Mixed Lymphocyte Reaction (MLR) assay; (d) increases interferon-y production in an MLR assay; (e) increases IL-2 secretion in an MLR assay; (f) binds to both human PD-1 and cynomolgus monkey PD-1; (g) inhibits the binding of PD-L1 and/or PD-L2 to PD-1; (h) stimulates antigen-specific memory responses; (i) stimulates Ab responses; and/or (j) inhibits tumor cell growth in vivo. Anti-PD-1 antibodies usable in the present invention include, for example, monoclonal antibodies (mAbs) that bind specifically to PD-1 and exhibit at least one, at least two, at least three, at least four or at least five of the preceding characteristics.

Exemplary anti-PD-1 antibodies also include, but are not limited to, mouse, humanized, human, chimeric, and engineered antibodies that comprise, for example, one or more of the CDR sequences described herein. In some embodiments, an anti-PD-1 antibody comprises a heavy chain variable region described herein. In some embodiments, an anti-PD-1 antibody comprises a light chain variable region described herein. In some embodiments, an anti-PD-1 antibody comprises a heavy chain variable region described herein and a light chain variable region described herein. In some embodiments, an anti-PD-1 antibody comprises heavy chain CDR1, CDR2, and CDR3 described herein, e.g., comprising SEQ ID NOs: 105, 107, and 109. In some embodiments, an anti-PD-1 antibody comprises light chain CDR1, CDR2, and CDR3 described herein, e.g., comprising SEQ ID NOs: 112, 114, and 116. In some embodiments, an anti-PD-1 antibody comprises heavy chain CDR1, CDR2, and CDR3 described herein, e.g., comprising SEQ ID NOs: 105, 107, and 109, and light chain CDR1, CDR2, and CDR3 described herein, e.g., comprising SEQ ID NOs: 112, 114, and 116.

In some embodiments, an anti-PD-1 antibody comprises heavy chain CDR1, CDR2, and CDR3 comprising SEQ ID NOs: 105, 107, and 109 respectively. In some embodiments, an anti-PD-1 antibody comprises light chain CDR1, CDR2, and CDR3 comprising SEQ ID NOs: 112, 114, and 116, respectively. In some embodiments, the anti-PD-1 antibody comprises a heavy chain variable region comprising SEQ ID NO: 100. In some embodiments, the anti-PD-1 antibody comprises a light chain variable region comprising SEQ ID NO: 102. In some embodiments, the anti-PD-1 antibody comprises a heavy chain variable region comprising SEQ ID NO: 100 and a light chain variable region comprising SEQ ID NO: 102. In some embodiments, the anti-PD-1 antibody comprises a heavy chain constant region comprising SEQ ID NO: 101 and/or a light chain constant region comprising SEQ ID NO: 103.

Further Exemplary Anti-PD-1 Antibodies

In some embodiments, an anti-PD-1 antibody comprises a heavy chain comprising a variable region sequence that is at least 90%, at least 91%, at least 92%, at least 93%, at least 94%, at least 95%, at least 96%, at least 97%, at least 98%, or at least 99% identical to SEQ ID NOs:100, wherein the antibody binds PD-1. In some embodiments, an anti-PD-1 antibody comprises a light chain comprising a variable region sequence that is at least 90%, at least 91%, at least 92%, at least 93%, at least 94%, at least 95%, at least 96%, at least 97%, at least 98%, or at least 99% identical to SEQ ID NOs:102, wherein the antibody binds PD-1. In some embodiments, an anti-PD-1 antibody comprises a heavy chain comprising a variable region sequence that is at least 90%, at least 91%, at least 92%, at least 93%, at least 94%, at least 95%, at least 96%, at least 97%, at least 98%, or at least 99% identical to SEQ ID NOs:100; and a light chain comprising a variable region sequence that is at least 90%, at least 91%, at least 92%, at least 93%, at least 94%, at least 95%, at least 96%, at least 97%, at least 98%, or at least 99% identical to SEQ ID NOs:102; wherein the antibody binds PD-1.

In some embodiments, an anti-PD-1 antibody comprises at least one of the CDRs discussed herein. That is, in some embodiments, an anti-PD-1 antibody comprises at least one CDR selected from a heavy chain CDR1 discussed herein, a heavy chain CDR2 discussed herein, a heavy chain CDR3 discussed herein, a light chain CDR1 discussed herein, a light chain CDR2 discussed herein, and a light chain CDR3 discussed herein. Further, in some embodiments, an anti-PD-1 antibody comprises at least one mutated CDR based on a CDR discussed herein, wherein the mutated CDR comprises 1, 2, 3, or 4 amino acid substitutions relative to the CDR discussed herein. In some embodiments, one or more of the amino acid substitutions are conservative amino acid substitutions. One skilled in the art can select one or more suitable conservative amino acid substitutions for a particular CDR sequence, wherein the suitable conservative amino acid substitutions are not predicted to significantly alter the binding properties of the antibody comprising the mutated CDR.

In one embodiment, the anti-PD-1 Ab is nivolumab. Nivolumab (also known as “Opdivo®”; formerly designated 5C4, BMS-936558, MDX-1106, or ONO-4538, is a fully human IgG4 (S228P) (EU numbering; S228P is S241P under Kabat numbering) anti-PD-1 antibody that selectively prevents interaction with PD-1 ligands (PD-L1 and PD-L2), thereby blocking the down-regulation of antitumor T-cell functions (U.S. Pat. No. 8,008,449; Wang et al., 2014 Cancer Immunol Res. 2(9):846-56).

In another embodiment, the anti-PD-1 Ab is pembrolizumab. Pembrolizumab (also known as “Keytruda®”, lambrolizumab, and MK-3475) is a humanized monoclonal IgG4 anti-PD-1 antibody. Pembrolizumab is described, for example, in U.S. Pat. No. 8,900,587; see also www (dot) cancer (dot) gov (slash) drugdictionary?cdrid=695789 (last accessed: Mar. 27, 2017). Pembrolizumab has been approved by the FDA for the treatment of relapsed or refractory melanoma.

In other embodiments, the anti-PD-1 Ab is MEDI0608 (formerly AMP-514). MEDI0608 is described, for example, in US Pat. No. 8,609,089,B2 or in www (dot) cancer (dot) gov (slash) drugdictionary?cdrid=756047 (last accessed Mar. 27, 2017).

Anti-PD-1 Abs usable in the disclosed methods also include isolated Abs that bind specifically to human PD-1 and cross-compete for binding to human PD-1 with nivolumab (see, e.g., U.S. Pat. No. 8,008,449; WO 2013/173223). The ability of Abs to cross-compete for binding to an antigen indicates that these Abs bind to the same epitope region of the antigen and sterically hinder the binding of other cross-competing Abs to that particular epitope region. These cross-competing Abs are expected to have functional properties similar to those of nivolumab by virtue of their binding to the same epitope region of PD-1. Cross-competing Abs can be readily identified based on their ability to cross-compete with nivolumab in standard PD-1 binding assays such as Biacore® analysis, ELISA assays or flow cytometry (see, e.g., WO 2013/173223).

In certain embodiments, the Abs that cross-compete for binding to human PD-1 with, or bind to the same epitope region of PD-1 as, nivolumab are mAbs. For administration to human subjects, these cross-competing Abs can be chimeric Abs, or can be humanized or human Abs.

Anti-PD-1 Abs usable in the methods of the disclosed invention also include antigen-binding portions of the above Abs, such as: (i) a Fab fragment, a monovalent fragment consisting of the V_{L}, V_{H}, C_{L} and C_{HI} domains; (ii) a F(ab′)2 fragment, a bivalent fragment comprising two Fab fragments linked by a disulfide bridge at the hinge region; (iii) a Fd fragment consisting of the V_{H} and C_{HI} domains; and (iv) a Fv fragment consisting of the V_{L} and V_{H} domains of a single arm of an Ab.

### Exemplary Antibody or Polypeptide Conjugates

In some embodiments, an antibody herein is conjugated to a label and/or a cytotoxic agent. As used herein, a label is a moiety that facilitates detection of the antibody or polypeptide and/or facilitates detection of a molecule to which the antibody or polypeptide binds. Nonlimiting exemplary labels include, but are not limited to, radioisotopes, fluorescent groups, enzymatic groups, chemiluminescent groups, biotin, epitope tags, metal-binding tags, etc. One skilled in the art can select a suitable label according to the intended application.

As used herein, a cytotoxic agent is a moiety that reduces the proliferative capacity of one or more cells. A cell has reduced proliferative capacity when the cell becomes less able to proliferate, for example, because the cell undergoes apoptosis or otherwise dies, the cell fails to proceed through the cell cycle and/or fails to divide, the cell differentiates, etc. Nonlimiting exemplary cytotoxic agents include, but are not limited to, radioisotopes, toxins, and chemotherapeutic agents. One skilled in the art can select a suitable cytotoxic according to the intended application.

In some embodiments, a label and/or a cytotoxic agent is conjugated to an antibody using chemical methods in vitro. Nonlimiting exemplary chemical methods of conjugation are known in the art, and include services, methods and/or reagents commercially available from, e.g., Thermo Scientific Life Science Research Produces (formerly Pierce; Rockford, Ill.), Prozyme (Hayward, Calif.), SACRI Antibody Services (Calgary, Canada), AbD Serotec (Raleigh, N.C.), etc. In some embodiments, when a label and/or cytotoxic agent is a polypeptide, the label and/or cytotoxic agent can be expressed from the same expression vector with at least one antibody or polypeptide chain to produce a polypeptide comprising the label and/or cytotoxic agent fused to an antibody or polypeptide molecule.

### Exemplary Leader Sequences

In order for some secreted proteins to express and secrete in large quantities, a leader sequence from a heterologous protein may be desirable. In some embodiments, a leader sequence is selected from SEQ ID NOs: 3 and 4, which are light chain and heavy chain leader sequences, respectively. In some embodiments, employing heterologous leader sequences may be advantageous in that a resulting mature polypeptide may remain unaltered as the leader sequence is removed in the ER during the secretion process. The addition of a heterologous leader sequence may be required to express and secrete some proteins.

Certain exemplary leader sequence sequences are described, e.g., in the online Leader sequence Database maintained by the Department of Biochemistry, National University of Singapore. See Choo et al., BMC Bioinformatics, 6: 249 (2005); and PCT Publication No. WO 2006/081430.

### Therapeutic Compositions and Methods

Methods of Treating Cancer

In some embodiments, methods for treating cancer are provided, comprising administering an effective amount of an anti-GITR antibody and either: (i) an effective amount of anti-CSF1R antibody or (ii) an effective amount of an anti-PD-1 antibody. In some embodiments, methods for treating cancer are provided, comprising administering an effective amount of an anti-GITR antibody and an effective amount of each of an anti-CSF1R antibody and an anti-PD-1 antibody. In some embodiments, the anti-GITR antibody and the anti-CSF1R antibody and/or anti-PD-1 antibody are administered concurrently. In some embodiments, the anti-GITR antibody and the anti-CSF1R antibody and/or anti-PD-1 antibody are administered sequentially. In each method of treatment embodiment herein, any of the anti-GITR antibodies, anti-CSF1R antibodies, and/or anti-PD-1 antibodies described in the preceding sections of this disclosure may be administered.

In some embodiments, at least one, at least two, at least three doses, at least five doses, or at least ten doses of anti-GITR antibody is administered prior to administration of an anti-PD-1 antibody or anti-CSF1R antibody. In some embodiments, at least one, at least two, at least three doses, at least five doses, or at least ten doses of a anti-PD-1 antibody or anti-CSF1R antibody is administered prior to administration of anti-GITR antibody. In some embodiments, the last dose of anti-GITR antibody is administered at least one, two, three, five, days or ten, or one, two, three, five, twelve, or twenty four weeks prior to the first dose of anti-PD-1 antibody or anti-CSF1R antibody. In some other embodiment, the last dose of anti-PD-1 antibody or anti-CSF1R antibody is administered at least one, two, three, five, days or ten, or one, two, three, five, twelve, or twenty four weeks prior to the first dose of anti-GITR antibody. In some embodiments, a subject has received, or is receiving, anti-PD-1 antibody therapy or anti-CSF1R antibody therapy and anti-GITR antibody is added to the therapeutic regimen. In other embodiments, a subject has received, or is receiving, anti-GITR antibody therapy and anti-PD-1 antibody therapy or anti-CSF1R antibody therapy is added to the therapeutic regimen.

In some embodiments where each of an anti-GITR antibody, anti-CSF1R antibody, and an anti-PD-1 antibody is administered, at least one, at least two, at least three doses, at least five doses, or at least ten doses of anti-CSF1R antibody are administered prior to administration of anti-PD-1 antibody. In some embodiments, at least one, at least two, at least three doses, at least five doses, or at least ten doses of anti-PD-1 antibody are administered prior to administration of anti-CSF1R antibody. In some embodiments, the last dose of anti-CSF1R antibody is administered at least one, two, three, five, days or ten, or one, two, three, five, twelve, or twenty four weeks prior to the first dose of anti-PD-1 antibody. In some other embodiments, the last dose of anti-PD-1 antibody is administered at least one, two, three, five, days or ten, or one, two, three, five, twelve, or twenty four weeks prior to the first dose of anti-CSF1R antibody. In some embodiments, a subject has received, or is receiving, anti-PD-1 antibody therapy and an anti-GITR antibody therapy and anti-CSF1R antibody are added to the therapeutic regimen. In other embodiments, a subject has received, or is receiving, anti-CSF1R antibody therapy and an anti-PD-1 antibody therapy and anti-GITR antibody therapy are added to the therapeutic regimen.

In some embodiments, the combination of anti-GITR antibody and either or both of anti-PD-1 antibody and anti-CSF1R antibody is used for cancer treatment. In some embodiments, the cancer is selected from squamous cell cancer, small-cell lung cancer, pituitary cancer, esophageal cancer, astrocytoma, soft tissue sarcoma, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, renal cancer, liver cancer, prostate cancer, vulval cancer, thyroid cancer, hepatic carcinoma, brain cancer, endometrial cancer, testis cancer, cholangiocarcinoma, gallbladder carcinoma, gastric cancer, melanoma, and various types of head and neck cancer. In some embodiments, lung cancer is non-small cell lung cancer or lung squamous cell carcinoma. In some embodiments, leukemia is acute myeloid leukemia or chronic lymphocytic leukemia. In some embodiments, breast cancer is breast invasive carcinoma. In some embodiments, ovarian cancer is ovarian serous cystadenocarcinoma. In some embodiments, kidney cancer is kidney renal clear cell carcinoma. In some embodiments, colon cancer is colon adenocarcinoma. In some embodiments, bladder cancer is bladder urothelial carcinoma. In some embodiments, the cancer is selected from bladder cancer, cervical cancer (such as squamous cell cervical cancer), head and neck squamous cell carcinoma, rectal adenocarcinoma, non-small cell lung cancer, endometrial cancer, prostate adenocarcinoma, colon cancer, ovarian cancer (such as serous epithelial ovarian cancer), and melanoma.

In some embodiments, the subject has previously received treatment with a PD-1/PD-L1 inhibitor. In some such cases, the subject has been refractory to treatment with the PD-1/PD-L1 inhibitor. In some embodiments of the methods described herein, the subject is an anti-PD-1 antibody inadequate responder (i.e., has been refractory to treatment with an anti-PD-1 antibody). A subject who is an anti-PD-1 antibody inadequate responder, may have previously responded to a anti-PD-1 antibody, but may have become less responsive to the anti-PD-1 antibody, or the subject may have never responded to the anti-PD-1 antibody. Inadequate response to a anti-PD-1 antibody means that aspects of the condition that would be expected to improve following a standard dose of the anti-PD-1 antibody do not improve, and/or improvement only occurs if greater than a standard dose is administered. In some embodiments, an anti-PD-1 antibody inadequate responder has experienced, or is experiencing, an inadequate response to the anti-PD-1 antibody after receiving a standard dose for at least two weeks, at least three weeks, at least four weeks, at least six weeks, or at least twelve weeks. A “standard” dose is determined by a medical professional, and may depend on the subject's age, weight, healthy history, severity of disease, the frequency of dosing, etc. In some embodiments, an anti-PD-1 antibody inadequate responder has experienced, or is experiencing, an inadequate response to an anti-PD-1 antibody and/or an anti-PD-L1 antibody. In some embodiments, an anti-PD-1 antibody inadequate responder has experienced, or is experiencing, an inadequate response to a different type of PD-1/PD-L1 inhibitor such as an anti-PD-L1 antibody. In some embodiments, an anti-PD-1 antibody inadequate responder has experienced, or is experiencing, an inadequate response to an anti-PD-1 antibody selected from nivolumab and pembrolizumab.

In some embodiments, methods for treating pancreatic cancer are provided, comprising administering an effective amount of an anti-GITR antibody and an effective amount of anti-CSF1R antibody. In some embodiments, an effective amount of an anti-PD-1 antibody is also administered. In some embodiments, the anti-GITR antibody and the anti-CSF1R antibody are administered concurrently. In some embodiments, the anti-GITR antibody and the anti-CSF1R antibody are administered sequentially. In any of these treatment methods, any of the anti-GITR antibodies and anti-CSF1R antibodies, and optionally anti-PD-1 antibodies, described in the preceding sections of this disclosure may be administered.

In some embodiments, an anti-GITR antibody and an anti-CSF1R antibody (and optionally an anti-PD-1 antibody) or an anti-GITR antibody and an anti-PD-1 antibody may be administered with one or more chemotherapy agents. In some such embodiments, the chemotherapy agent is selected from gemcitabine, nab-paclitaxel, leukovorin (folinic acid), 5-fluorouracil (5-FU), irinotecan, and oxaliplatin. In some such embodiments, the anti-GITR antibody and anti-CSF1R antibody (and optionally the anti-PD-1 antibody) is administered with FOLFIRINOX, which is a chemotherapy regime that includes a combination of leukovorin, 5-FU, irinotecan (such as liposomal irinotecan injection), and oxaliplatin. In some embodiments, an anti-GITR antibody and an anti-CSF1R (and optionally an anti-PD-1 antibody) or an anti-GITR antibody and anti-PD-1 antibody may be administered with gemcitabine-based chemotherapy. In some embodiments, an anti-GITR antibody and an anti-CSF1R (and optionally an anti-PD-1 antibody) or an anti-GITR antibody and anti-PD-1 antibody may be administered with at least one agent selected from (a) gemcitabine; (b) gemcitabine and nab-paclitaxel; and (c) FOLFIRINOX. In some such embodiments, the at least one agent is gemcitabine.

In some embodiments for treating pancreatic cancer, for example, an anti-GITR antibody and an anti-CSF1R antibody (and optionally an anti-PD-1 antibody) may be administered with one or more chemotherapy agents. In some such embodiments for treating pancreatic cancer, the chemotherapy agent is selected from gemcitabine, nab-paclitaxel, leukovorin, 5-FU), irinotecan, and oxaliplatin. In some such embodiments, the anti-GITR antibody and anti-CSF1R antibody (and optionally the anti-PD-1 antibody) is administered with FOLFIRINOX, which is a chemotherapy regime that includes a combination of leukovorin, 5-FU, irinotecan (such as liposomal irinotecan injection), and oxaliplatin. In some embodiments for treating pancreatic cancer, an anti-GITR antibody and an anti-CSF1R antibody (and optionally an anti-PD-1 antibody) may be administered with gemcitabine-based chemotherapy. In some embodiments for treating pancreatic cancer, an anti-GITR antibody and anti-CSF1R antibody (and optionally an anti-PD1 antibody) may be administered with at least one agent selected from (a) gemcitabine; (b) gemcitabine and nab-paclitaxel; and (c) FOLFIRINOX. In some such embodiments for treating pancreatic cancer, the at least one agent is gemcitabine.

Routes of Administration, Carriers, and Dosages

In various embodiments, polypeptides or antibodies may be administered in vivo by various routes, including, but not limited to, oral, intra-arterial, parenteral, intranasal, intravenous, intramuscular, intracardiac, intraventricular, intratracheal, buccal, rectal, intraperitoneal, intradermal, topical, transdermal, and intrathecal, or otherwise by implantation or inhalation. The subject compositions may be formulated into preparations in solid, semi-solid, liquid, or gaseous forms; including, but not limited to, tablets, capsules, powders, granules, ointments, solutions, suppositories, enemas, injections, inhalants, and aerosols.

In various embodiments, compositions comprising antibodies and other polypeptides are provided in formulations with a wide variety of pharmaceutically acceptable carriers (see, e.g., Gennaro, Remington: The Science and Practice of Pharmacy with Facts and Comparisons: Drugfacts Plus, 20^{th} ed. (2003); Ansel et al., Pharmaceutical Dosage Forms and Drug Delivery Systems, 7^{th} ed., Lippencott Williams and Wilkins (2004); Kibbe et al., Handbook of Pharmaceutical Excipients, 3^{rd} ed., Pharmaceutical Press (2000)). Various pharmaceutically acceptable carriers, which include vehicles, adjuvants, and diluents, are available. Moreover, various pharmaceutically acceptable auxiliary substances, such as pH adjusting and buffering agents, tonicity adjusting agents, stabilizers, wetting agents and the like, are also available. Non-limiting exemplary carriers include saline, buffered saline, dextrose, water, glycerol, ethanol, and combinations thereof.

In various embodiments, compositions comprising antibodies and other polypeptides may be formulated for injection, including subcutaneous administration, by dissolving, suspending, or emulsifying them in an aqueous or nonaqueous solvent, such as vegetable or other oils, synthetic aliphatic acid glycerides, esters of higher aliphatic acids, or propylene glycol; and if desired, with conventional additives such as solubilizers, isotonic agents, suspending agents, emulsifying agents, stabilizers and preservatives. In various embodiments, the compositions may be formulated for inhalation, for example, using pressurized acceptable propellants such as dichlorodifluoromethane, propane, nitrogen, and the like. The compositions may also be formulated, in various embodiments, into sustained release microcapsules, such as with biodegradable or non-biodegradable polymers. A non-limiting exemplary biodegradable formulation includes poly lactic acid-glycolic acid polymer. A non-limiting exemplary non-biodegradable formulation includes a polyglycerin fatty acid ester. Certain methods of making such formulations are described, for example, in EP 1 125 584 A1.

Pharmaceutical packs and kits comprising one or more containers, each containing one or more doses of an antibody or combinations of antibodies are also provided. In some embodiments, a unit dosage is provided wherein the unit dosage contains a predetermined amount of a composition comprising an antibody or combination of antibodies, with or without one or more additional agents. In some embodiments, such a unit dosage is supplied in single-use prefilled syringe for injection, for example, or as a kit. In various embodiments, the composition contained in the unit dosage may comprise saline, sucrose, or the like; a buffer, such as phosphate, or the like; and/or be formulated within a stable and effective pH range. Alternatively, in some embodiments, the composition may be provided as a lyophilized powder that may be reconstituted upon addition of an appropriate liquid, for example, sterile water. In some embodiments, the composition comprises one or more substances that inhibit protein aggregation, including, but not limited to, sucrose and arginine. In some embodiments, a composition of the invention comprises heparin and/or a proteoglycan.

Pharmaceutical compositions are administered in an amount effective for treatment of the specific indication. The therapeutically effective amount is typically dependent on the weight of the subject being treated, his or her physical or health condition, the extensiveness of the condition to be treated, or the age of the subject being treated.

In some embodiments, an anti-PD-1 antibody is administered at a dose of 0.5 to 10 mg/kg, such as 0.5, 1, 2, 3, 4, 5, 6, 7, 8, 9, or 10 mg/kg. In some embodiments, an anti-PD-1 antibody is administered at a dose of 1 to 4 mg/kg, such as 1, 2, 3, or 4 mg/kg. In some embodiments, an anti-PD-1 antibody may be administered every week, every 2 weeks, every 3 weeks, or every 4 weeks. For example, in some embodiments, in which the anti-PD-1 antibody comprises nivolumab, the nivolumab may be administered at a dose of 3 mg/kg. In some such embodiments, the nivolumab may be administered at a dose of 3 mg/kg every week, every 2 weeks, every 3 weeks, or every 4 weeks. In some such embodiments, the nivolumab may be administered at a dose of 3 mg/kg every 2 weeks.

In some embodiments, an anti-CSF1R antibody is administered at a dose of 0.3 to 10 mg/kg, 0.5 to 10 mg/kg, 0.5 to 5 mg/kg, or 1 to 5 mg/kg body weight, such as at 0.3, 0.5, 1, 2, 3, 4, 5, or 10 mg/kg. In some embodiments, an anti-CSF1R antibody may be administered every week, every 2 weeks, every 3 weeks, or every 4 weeks. In some embodiments, an anti-CSF1R antibody may be administered at 1, 2, 3, or 4 mg/kg every 2 weeks. In some such embodiments, an anti-CSF1R antibody may be administered at 1, 2, 3, or 4 mg/kg every 2 weeks.

In certain embodiments, the dose of an anti-PD-1 antibody or anti-CSF1R antibody is a fixed dose in a pharmaceutical composition. In other embodiments, the method of the present invention can be used with a flat dose (a dose given to a patient irrespective of the body weight of the patient). For example, a flat dose of the anti-PD-1 antibody nivolumab can be 240mg. In some embodiments, nivolumab may be administered at 240 mg every 2 weeks. For example, a flat dose of the anti-PD-1 antibody pembrolizumab can be 200 mg. In some embodiments, pembrolizumab may be administered at 200 mg every 3 weeks.

A dosage of an anti-CSF1R antibody or anti-PD-1 antibody that is significantly lower than the approved therapeutic dose for monotherapy may be regarded as subtherapeutic. In certain embodiments, the anti-PD-1 antibody is administered at a dosage of 0.1, 0.3, 0.5, 1, 2, 3, 4, or 5 mg/kg, once every 2 weeks, once every 3 weeks, or once every 4 weeks. In certain embodiments, an anti-CSF1R antibody administered at a dosage of 0.1, 0.3, 0.5, 1, 2, 3, 4, 5, or 10 mg/kg, once every 2 weeks, once every 3 weeks, or once every 4 weeks. Some or all of the above doses may be considered subtherapeutic doses when compared to the approved therapeutic dose for monotherapy with the same antibody.

In certain embodiments, the anti-GITR antibody, anti-PD-1 antibody and/or anti-CSF1R antibody are formulated as a single composition. In other embodiments, they are formulated separately as different compositions. In some embodiments, the dose of the anti-CSF1R antibody or anti-GITR antibody or anti-PD-1 antibody is a fixed dose. In certain embodiments, the dose of the anti-GITR antibody, anti-CSF1R antibody or anti-PD-1 antibody is a flat dose, which is given to a patient irrespective of the body weight.

Combination with Other Therapies

Antibodies may be administered alone or with other modes of treatment. They may be provided before, substantially contemporaneously with, or after other modes of treatment, for example, surgery, chemotherapy, radiation therapy, or the administration of a biologic, such as another therapeutic antibody. In some embodiments, the cancer has recurred or progressed following a therapy selected from surgery, chemotherapy, and radiation therapy, or a combination thereof.

Combinations with Immune Stimulating Agents

In some embodiments, the combination treatments herein may be further combined with at least one immune stimulating agent. The term “immune stimulating agent” as used herein refers to a molecule that stimulates the immune system by either acting as an agonist of an immune-stimulatory molecule, including a co-stimulatory molecule, or acting as an antagonist of an immune inhibitory molecule, including a co-inhibitory molecule. An immune stimulating agent may be a biologic or a small molecule compound. Examples of biologic immune stimulating agents include, but are not limited to, antibodies, antibody fragments, fragments of receptor or ligand polypeptides, for example that block receptor-ligand binding, vaccines and cytokines.

In some embodiments, the at least one immune stimulating agent comprises an agonist of an immune stimulatory molecule, including a co-stimulatory molecule, while in some embodiments, the at least one immune stimulating agent comprises an antagonist of an immune inhibitory molecule, including a co-inhibitory molecule. In some embodiments, the at least one immune stimulating agent comprises an agonist of an immune-stimulatory molecule, including a co-stimulatory molecule, found on immune cells, such as T cells. In some embodiments, the at least one immune stimulating agent comprises an antagonist of an immune inhibitory molecule, including a co-inhibitory molecule, found on immune cells, such as T cells. In some embodiments, the at least one immune stimulating agent comprises an agonist of an immune stimulatory molecule, including a co-stimulatory molecule, found on cells involved in innate immunity, such as NK cells. In some embodiments, the at least one immune stimulating agent comprises an antagonist of an immune inhibitory molecule, including a co-inhibitory molecule, found on cells involved in innate immunity, such as NK cells. In some embodiments, the combination enhances the antigen-specific T cell response in the treated subject and/or enhances the innate immunity response in the subject.

In certain embodiments, an immune stimulating agent targets a stimulatory or inhibitory molecule that is a member of the immunoglobulin super family (IgSF). For example, an immune stimulating agent may be an agent that targets (or binds specifically to) another member of the B7 family of polypeptides. An immune stimulating agent may be an agent that targets or binds to a member of the TNF family of membrane bound ligands or a co-stimulatory or co-inhibitory receptor binding specifically to a member of the TNF family. Exemplary TNF and TNFR family members that may be targeted by the immune stimulating agents herein include CD40 and CD40L, OX-40, OX-40L, GITRL, CD70, CD27L, CD30, CD30L, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fn14, TWEAK, BAFFR, EDAR, XEDAR, TACI, APRIL, BCMA, LTβR, LIGHT, DcR3, HVEM, VEGI/TL1A, TRAMP/DR3, EDAR, EDA1, XEDAR, EDA2, TNFR1, Lymphotoxin α/TNFβ, TNFR2, TNFα, LTβR, Lymphotoxin α 1β2, FAS, FASL, RELT, DR6, TROY and NGFR.

In some embodiments, an immune stimulating agent may comprise (i) an antagonist of a protein that inhibits T cell activation (e.g., immune checkpoint inhibitor) such as CTLA4 (e.g. an anti-CTLA4 antibody, e.g. YERVOY (ipilimumab) or tremelimumab), LAG-3 (e.g. an anti-LAG-3 antibody, for example, BMS-986016 (WO10/19570, WO14/08218), or IMP-731 or IMP-321 (WO08/132601, WO09/44273), TIM3, Galectin 9, CEACAM-1, BTLA, CD69, Galectin-1, TIGIT, CD113, GPR56, VISTA, B7-H3 (e.g. MGA271 (WO11/109400)), B7-H4, 2B4, CD48, GARP, PD1H, LAIR1, TIM-1, TIM-4, and ILT4 and/or may comprise (ii)an agonist of a protein that stimulates T cell activation such as B7-2, CD28, 4-1BB (CD137) (e.g. a CD137 agonist antibody such as urelumab or PF-05082566 (WO12/32433)), 4-1BBL, ICOS, ICOS-L, OX40 (e.g. an OX40 agonist antibody, for example, MEDI-6383, MEDI-6469 or MOXR0916 (RG7888; WO06/029879)), OX40L, GITRL, CD70, CD27 (e.g. an agonistic CD27 antibody such as varlilumab (CDX-1127)), CD40, CD40L, DR3 and CD28H. In some embodiments, the agonist of a protein that stimulates T cell activation is an antibody.

In some embodiments, an immune stimulating agent may comprise an agent that inhibits or is an antagonist of a cytokine that inhibits T cell activation (e.g., IL-6, IL-10, TGF-β, VEGF, and other immunosuppressive cytokines), and in some embodiments an immune stimulating agent may comprise an agent that is an agonist of a cytokine, such as IL-2, IL-7, IL-12, IL-15, IL-21 and IFNα (e.g., the cytokine itself) that stimulates T cell activation. TGF-β inhibitors include, e.g., GC1008, LY2157299, TEW7197 and IMC-TR1. In some embodiments, immune stimulating agents may comprise an antagonist of a chemokine, such as CXCR2 (e.g., MK-7123), CXCR4 (e.g. AMD3100), CCR2, or CCR4 (mogamulizumab).

In some embodiments, the at least one immune stimulating agent comprises a Toll-like receptor agonist, e.g., a TLR2/4 agonist (e.g., Bacillus Calmette-Guerin); a TLR7 agonist (e.g., Hiltonol or Imiquimod); a TLR7/8 agonist (e.g., Resiquimod); or a TLR9 agonist (e.g., CpG7909).

In some embodiments, immune stimulating agents may include antagonists of inhibitory receptors on NK cells or agonists of activating receptors on NK cells. In some embodiments, the at least one immune stimulating agent is an antagonist of KIR, e.g. the antibody lirilumab.

Immune stimulating agents may also include agents that enhance tumor antigen presentation, e.g., dendritic cell vaccines, GM-CSF secreting cellular vaccines, CpG oligonucleotides,and imiquimod, or therapies that enhance the immunogenicity of tumor cells (e.g., anthracyclines).

Immune stimulating agents may also include certain vaccines such as mesothelin-targeting vaccines or attenuated listeria cancer vaccines, such as CRS-207.

Immune stimulating agents may also comprise agents that deplete or block Treg cells, such as agents that specifically bind to CD25.

Immune stimulating agents may also comprise agents that inhibit a metabolic enzyme such as indoleamine dioxigenase (IDO), dioxigenase, arginase, or nitric oxide synthetase. IDO antagonists include, for example, INCB-024360 (WO2006/122150, WO07/75598, WO08/36653, WO08/36642), indoximod, NLG-919 (WO09/73620, WO09/1156652, WO11/56652, WO12/142237) and F001287.

Immune stimulating agents may also comprise agents that inhibit the formation of adenosine or inhibit the adenosine A2A receptor.

Immune stimulating agents may also comprise agents that reverse/prevent T cell energy or exhaustion and agents that trigger an innate immune activation and/or inflammation at a tumor site.

The treatment combinations can also be further combined in a combinatorial approach that targets multiple elements of the immune pathway, such as one or more of the following: at least one agent that enhances tumor antigen presentation (e.g., dendritic cell vaccine, GM-C SF secreting cellular vaccines, CpG oligonucleotides, imiquimod); at least one agent that inhibits negative immune regulation e.g., by inhibiting CTLA4 pathway and/or depleting or blocking Treg or other immune suppressing cells; a therapy that stimulates positive immune regulation, e.g., with agonists that stimulate the CD-137 and/or OX-40 pathway and/or stimulate T cell effector function; at least one agent that increases systemically the frequency of anti-tumor T cells; a therapy that depletes or inhibits Tregs, such as Tregs in the tumor, e.g., using an antagonist of CD25 (e.g., daclizumab) or by ex vivo anti-CD25 bead depletion; at least one agent that impacts the function of suppressor myeloid cells in the tumor; a therapy that enhances immunogenicity of tumor cells (e.g., anthracyclines); adoptive T cell or NK cell transfer including genetically modified cells, e.g., cells modified by chimeric antigen receptors (CAR-T therapy); at least one agent that inhibits a metabolic enzyme such as indoleamine dioxigenase (IDO), dioxigenase, arginase or nitric oxide synthetase; at least one agent that reverses/prevents T cell anergy or exhaustion; a therapy that triggers an innate immune activation and/or inflammation at a tumor site; administration of immune stimulatory cytokines or blocking of immuno repressive cytokines.

For example, the at least one immune stimulating agent may comprise one or more agonistic agents that ligate positive costimulatory receptors; one or more antagonists (blocking agents) that attenuate signaling through inhibitory receptors, such as antagonists that overcome distinct immune suppressive pathways within the tumor microenvironment; one or more agents that increase systemically the frequency of anti-tumor immune cells, such as T cells, deplete or inhibit Tregs (e.g., by inhibiting CD25); one or more agents that inhibit metabolic enzymes such as IDO; one or more agents that reverse/prevent T cell anergy or exhaustion; and one or more agents that trigger innate immune activation and/or inflammation at tumor sites.

In some embodiments the at least one immune stimulating agent comprises a PD-1/PD-L1 inhibitor other than a PD-1 antibody. For example, in some embodiments the immune stimulating agent comprises a PD-L1 binding antibody. In some embodiments related to combination treatments with anti-GITR antibodies and anti-CSF1R antibodies, the immune stimulating agent does not comprise an anti-PD-1 antibody. In some embodiments related to combination treatments with anti-GITR antibodies and anti-CSF1R antibodies, the immune stimulating agent does not comprise a PD-1/PD-L1 inhibitor. In some embodiments related to combination treatments with anti-GITR antibodies and anti-CSF1R antibodies, the immune stimulating agent does not comprise a molecule binding to CSF1R. In some embodiments related to combination treatments with anti-GITR antibodies and anti-PD-1 antibodies, the immune stimulating agent does not comprise a molecule binding to CSF1R. In some embodiments related to combination treatments with anti-GITR antibodies and anti-PD-1 antibodies, the immune stimulating agent does not comprise a PD-1/PD-L1 inhibitor. In some embodiments herein, the immune stimulating agent does not comprise a molecule binding to GITR.

### Other Combination Therapies

For treatment of cancer, as discussed herein, the antibodies may be administered in conjunction with one or more additional anti-cancer agents, such as the chemotherapeutic agent, growth inhibitory agent, anti-angiogenesis agent and/or anti-neoplastic composition. Nonlimiting examples of chemotherapeutic agents, growth inhibitory agents, anti-angiogenesis agents, anti-cancer agents, and anti-neoplastic compositions that can be used in combination with the antibodies of the present invention are as follows.

A “chemotherapeutic agent” is a chemical compound useful in the treatment of cancer. Examples of chemotherapeutic agents include, but are not limited to, alkylating agents such as thiotepa and Cytoxan® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, Adriamycin® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., Taxol® paclitaxel (Bristol-Myers Squibb Oncology, Princeton, N.J.), Abraxane® Cremophor-free, albumin-engineered nanoparticle formulation of paclitaxel (American Pharmaceutical Partners, Schaumberg, Ill.), and Taxotere® doxetaxel (Rhône-Poulenc Rorer, Antony, France); chloranbucil; Gemzar® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; Navelbine® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above.

Further nonlimiting exemplary chemotherapeutic agents include anti-hormonal agents that act to regulate or inhibit hormone action on cancers such as anti-estrogens and selective estrogen receptor modulators (SERMs), including, for example, tamoxifen (including Nolvadex® tamoxifen), raloxifene, droloxifene, 4-hydroxytamoxifen, trioxifene, keoxifene, LY117018, onapristone, and Fareston® toremifene; aromatase inhibitors that inhibit the enzyme aromatase, which regulates estrogen production in the adrenal glands, such as, for example, 4(5)-imidazoles, aminoglutethimide, Megase® megestrol acetate, Aromasin® exemestane, formestanie, fadrozole, Rivisor® vorozole, Femara® letrozole, and Arimidex® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF expression inhibitor (e.g., Angiozyme® ribozyme) and a HER2 expression inhibitor; vaccines such as gene therapy vaccines, for example, Allovectin® vaccine, Leuvectin® vaccine, and Vaxid® vaccine; Proleukin® rIL-2; Lurtotecan® topoisomerase 1 inhibitor; Abarelix® rmRH; and pharmaceutically acceptable salts, acids or derivatives of any of the above.

In some embodiments, an anti-GITR antibody, anti-CSF1R and/or anti-PD-1 antibody may be further administered with gemcitabine-based chemotherapy in which one or more chemotherapy agents including gemcitabine or including gemcitabine and nab-paclitaxel are administered. In some such embodiments, an anti-GITR antibody, anti-CSF1R and/or anti-PD-1 antibody may be administered with at least one chemotherapy agent selected from gemcitabine, nab-paclitaxel, leukovorin (folinic acid), 5-fluorouracil (5-FU), irinotecan, and oxaliplatin. FOLFIRINOX is a chemotherapy regime comprising leukovorin, 5-FU, irinotecan (such as liposomal irinotecan injection), and oxaliplatin. In some embodiments, an an anti-GITR antibody, anti-CSF1R and/or anti-PD-1 antibody may be further administered with gemcitabine-based chemotherapy. In some embodiments, the anti-GITR antibody, anti-CSF1R and/or anti-PD-1 antibody may be further administered with at least one agent selected from (a) gemcitabine; (b) gemcitabine and nab-paclitaxel; and (c) FOLFIRINOX. In some embodiments, the at least one agent is gemcitabine. In some such embodiments, the cancer to be treated is pancreatic cancer.

An “anti-angiogenesis agent” or “angiogenesis inhibitor” refers to a small molecular weight substance, a polynucleotide (including, e.g., an inhibitory RNA (RNAi or siRNA)), a polypeptide, an isolated protein, a recombinant protein, an antibody, or conjugates or fusion proteins thereof, that inhibits angiogenesis, vasculogenesis, or undesirable vascular permeability, either directly or indirectly. It should be understood that the anti-angiogenesis agent includes those agents that bind and block the angiogenic activity of the angiogenic factor or its receptor. For example, an anti-angiogenesis agent is an antibody or other antagonist to an angiogenic agent, e.g., antibodies to VEGF-A (e.g., bevacizumab (Avastin®)) or to the VEGF-A receptor (e.g., KDR receptor or Flt-1 receptor), anti-PDGFR inhibitors such as Gleevec® (Imatinib Mesylate), small molecules that block VEGF receptor signaling (e.g., PTK787/ZK2284, SU6668, Sutent/SU11248 (sunitinib malate), AMG706, or those described in, e.g., international patent application WO 2004/113304). Anti-angiogenesis agents also include native angiogenesis inhibitors, e.g., angiostatin, endostatin, etc. See, e.g., Klagsbrun and D'Amore (1991) Annu. Rev. Physiol. 53:217-39; Streit and Detmar (2003) Oncogene 22:3172-3179 (e.g., Table 3 listing anti-angiogenic therapy in malignant melanoma); Ferrara & Alitalo (1999) Nature Medicine 5(12):1359-1364; Tonini et al. (2003) Oncogene 22:6549-6556 (e.g., Table 2 listing known anti-angiogenic factors); and, Sato (2003) Int. J. Clin. Oncol. 8:200-206 (e.g., Table 1 listing anti-angiogenic agents used in clinical trials).

A “growth inhibitory agent” as used herein refers to a compound or composition that inhibits growth of a cell (such as a cell expressing VEGF) either in vitro or in vivo. Thus, the growth inhibitory agent may be one that significantly reduces the percentage of cells (such as a cell expressing VEGF) in S phase. Examples of growth inhibitory agents include, but are not limited to, agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in Mendelsohn and Israel, eds., The Molecular Basis of Cancer, Chapter 1, entitled “Cell cycle regulation, oncogenes, and antineoplastic drugs” by Murakami et al. (W. B. Saunders, Philadelphia, 1995), e.g., p. 13. The taxanes (paclitaxel and docetaxel) are anticancer drugs both derived from the yew tree. Docetaxel (Taxotere®, Rhone-Poulenc Rorer), derived from the European yew, is a semisynthetic analogue of paclitaxel (Taxol®, Bristol-Myers Squibb). Paclitaxel and docetaxel promote the assembly of microtubules from tubulin dimers and stabilize microtubules by preventing depolymerization, which results in the inhibition of mitosis in cells.

The term “anti-neoplastic composition” refers to a composition useful in treating cancer comprising at least one active therapeutic agent. Examples of therapeutic agents include, but are not limited to, e.g., chemotherapeutic agents, growth inhibitory agents, cytotoxic agents, agents used in radiation therapy, anti-angiogenesis agents, cancer immunotherapeutic agents, apoptotic agents, anti-tubulin agents, and other-agents to treat cancer, such as anti-HER-2 antibodies, anti-CD20 antibodies, an epidermal growth factor receptor (EGFR) antagonist (e.g., a tyrosine kinase inhibitor), HER1/EGFR inhibitor (e.g., erlotinib (Tarceva®), platelet derived growth factor inhibitors (e.g., Gleevec® (Imatinib Mesylate)), a COX-2 inhibitor (e.g., celecoxib), interferons, cytokines, antagonists (e.g., neutralizing antibodies) that bind to one or more of the following targets ErbB2, ErbB3, ErbB4, PDGFR-beta, BlyS, APRIL, BCMA, or VEGF receptor(s), and other bioactive and organic chemical agents, etc. Combinations thereof are also included in the invention.

### EXAMPLES

The examples discussed below are intended to be purely exemplary of the invention and should not be considered to limit the invention in any way. The examples are not intended to represent that the experiments below are all or the only experiments performed. Efforts have been made to ensure accuracy with respect to numbers used (for example, amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Combination Therapy with an Anti-CSF1R Antibody and an Anti-GITR Antibody Suppresses Tumor Growth In Vivo Better than Either Therapeutic Alone

Seven week old female C57Bl/6 mice (Charles River Laboratories, Hollister, Calif.) were acclimated for one week. The murine colorectal carcinoma cell line MC38 was implanted subcutaneously over the right flank of the mice at 0.5×10⁶ cells/100 μl/mouse. Prior to inoculation, the cells were cultured for no more than three passages in RPMI-1640 medium supplemented with 10% heat-inactivated Fetal Bovine Serum (FBS), 2 mM L-Glutamine. Cells were grown at 37° C. in a humidified atmosphere with 5% CO₂. Upon reaching 80-85% confluence, cells were harvested and resuspended in a 1:1 mixture of serum-free RPMI-1640 and Matrigel at 5×10⁶ cells per milliliter.

Mice were monitored twice weekly following cell implantation for tumor growth. For tumor measurements, the length and width of each tumor was measured using calipers and volume was calculated according to the formula: Tumor volume (mm³)=(width (mm)×length (mm)²)/2. On Day 7 after inoculation, all tumors were measured, outliers were excluded, and mice were randomly assigned to treatment groups. For anti-CSF1R treatment, mice were administered a mouse surrogate antibody based on HuAb1 comprising a murine IgG1 at 30 mg/kg weekly, beginning on Day 7. For anti-GITR treatment, a mouse surrogate anti-GITR antibody was administered once at 2.5 mg/kg on Day 10. This antibody is a tetravalent molecule comprising two polypeptides, each comprising two llama sdAb-derived GITR binding domains linked to wild-type mouse IgG2a Fc regions. (See FIG. 3A for a representative architecture of such a tetravalent molecule.) As a control, mice were administered mouse IgG2a (Bioxcell, Clone C1.18.4) at 30 mg/kg weekly, beginning on Day 7. Therapeutics were administered via intraperitoneal (i.p.) injection. Mean tumor volume on Day 7 was approximately 110 mm³.

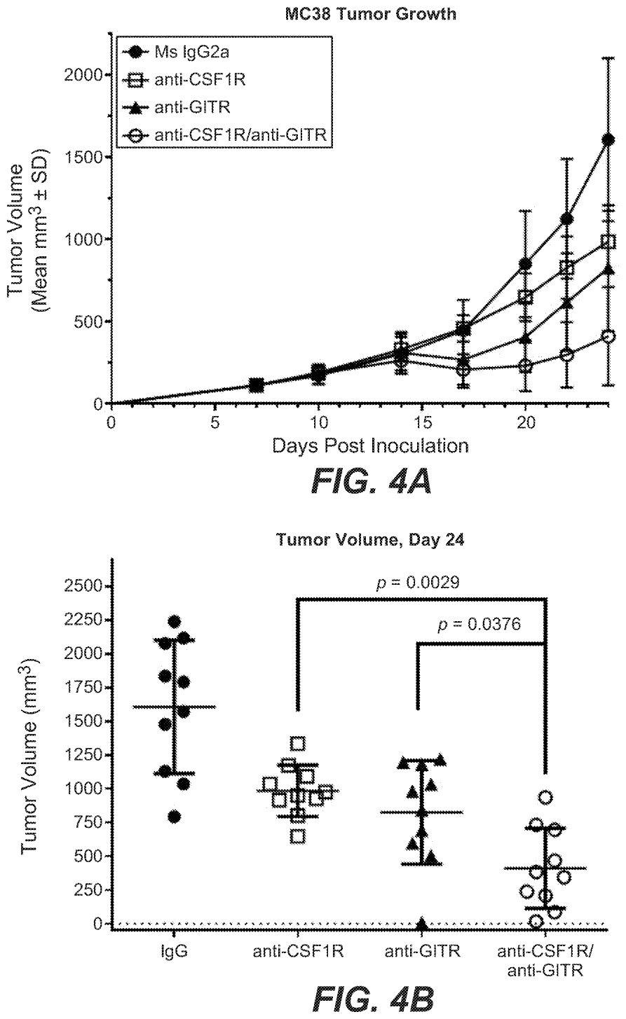

Tumors continued to be measured at least twice per week until tumor volume exceeded 10% of animal weight, or approximately 2000 mm³. The change in tumor size is shown by graphing individual tumors relative to the day upon which animals were inoculated with MC38 cells. As shown in FIG. 4B, the combination of anti-CSF1R with anti-GITR significantly reduced MC38 tumor volume compared to either anti-CSF1R or anti-GITR alone, as assessed by One-Way ANOVA comparing all groups to the combination group. In addition, treatment with anti-CSF1R, anti-GITR, or the combination significantly reduced tumor growth (p<0.05), as assessed by One-Way ANOVA compared to mouse IgG2a control (FIG. 4A).

### Example 2

### Combination Therapy with an Anti-GITR Antibody and an Anti-PD-1 Antibody Suppresses Tumor Growth In Vivo Better than Either Therapeutic Alone

Seven week old female C57Bl/6 mice were purchased from Charles River Laboratories (Hollister, Calif.) and were acclimated for one week before the start of the study. The murine colorectal carcinoma cell line MC38 was implanted subcutaneously over the right flank of the mice at 0.5×10⁶ cells/100 μl/mouse. Prior to inoculation, the cells were cultured for no more than three passages in RPMI-1640 medium supplemented with 10% heat-inactivated Fetal Bovine Serum (FBS), 2mM L-Glutamine. Cells were grown at 37° C. in a humidified atmosphere with 5% CO₂. Upon reaching 80-85% confluence, cells were harvested and resuspended in a 1:1 mixture of serum-free RPMI-1640 and Matrigel at 5 ×10⁶ cells per milliliter.

Mice were monitored twice weekly following cell implantation for tumor growth. For tumor measurements, the length and width of each tumor was measured using calipers and volume was calculated according to the formula: Tumor volume (mm³)=(width (mm)×length (mm)²)/2. On Day 9 after inoculation, all tumors were measured, outliers were excluded, and mice were randomly assigned to treatment groups.

For anti-GITR treatment, two different antibodies were tested: either a tetravalent anti-GITR antibody with a wild-type mouse IgG2a Fc as described in Example 1 above(WT Fc) or a tetravalent anti-GITR antibody with the same architecture as the WT Fc antibody, but with a mouse IgG2a Fc containing N297G and D265A substitutions designed to eliminate Fc effector function(Fc Silent). Either the WT Fc or the Fc Silent anti-GITR antibody was administered to the mice once on Day 9 (0.5 mg/kg for WT Fc and 2.5 mg/kg for Fc Silent). For anti-PD-1 treatment, mice were administered 5 mg/kg of antibody RMPI-14 containing a mouse IgG2a Fc with an N297A substitution, intended to eliminate Fc effector function, on Day 9 and Day 13. As a control, mice were administered mouse IgG2a (Bioxcell, Clone C1.18.4) at 5 mg/kg weekly on Day 9 and Day 13. Therapeutics were administered via intraperitoneal (i.p.) injection. Mean tumor volume on Day 9 was approximately 190 mm³. Tumors continued to be measured at least twice per week until tumor volume exceeded 10% of animal weight, or approximately 2000 mm³. The change in tumor size is shown by graphing individual tumor volume relative to the day upon which animals were inoculated with MC38 cells.

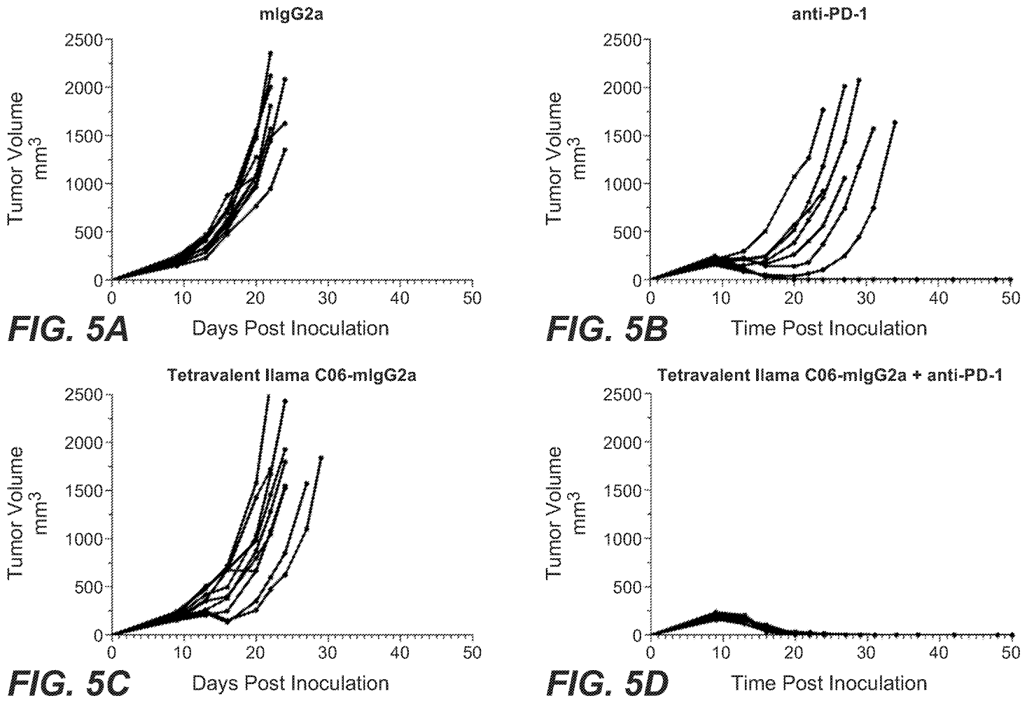

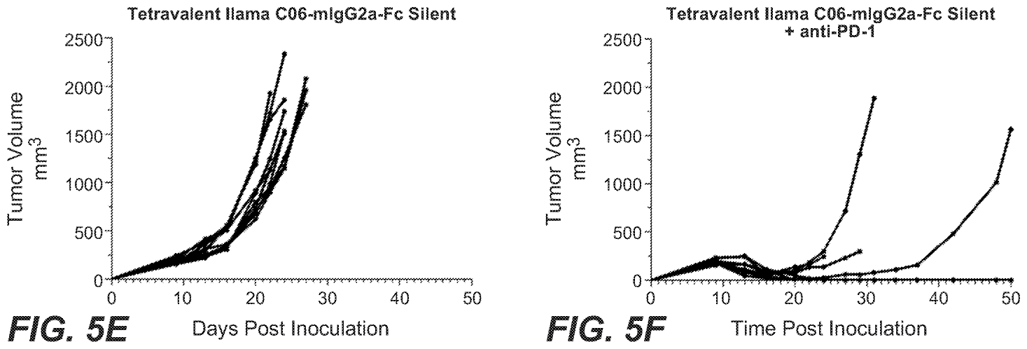

The combination of anti-GITR (WT Fc) with anti-PD-1 resulted in complete tumor regression in 100% of the treated animals (10 of 10 mice), as compared to anti-PD-1 treatment alone, which resulted in complete tumor regression in 30% of the treated animals (3 of 10). The combination of anti-GITR (Fc Silent) with anti-PD-1 resulted in complete tumor regression in 60% of treated animals (6 of 10). (See FIGS. 5A-5F.)

Although the anti-GITR (Fc Silent) antibody lacks Fc effector function, it is still highly potent in these experiments in combination with anti-PD-1, which may be due to the ability of the tetravalent molecule to trimerize and therefore agonize cell surface GITR.

### Example 3

### Combination Therapy with an Anti-GITR Antibody and Gemcitabine With and Without an Anti-CSF1R Antibody in a Murine Pancreatic Ductal Adenocarcinoma Cell (PDAC) Model

Eight week old female C57Bl/6 mice were purchased from Charles River Laboratories and were acclimated for up to two weeks before the start of the study. A murine pancreatic ductal adenocarcinoma cell (PDAC) line derived from Kras^{G12D}/p53^{-/-} transgenic mice was surgically implanted into the pancreas of the mice at 0.25×10⁶ cells/50 μl/mouse. Prior to inoculation, the cells were cultured for no more than three passages in DMEM medium supplemented with 10% heat-inactivated Fetal Bovine Serum (FBS). Cells were grown at 37° C. in a humidified atmosphere with 5% CO₂. Upon reaching 80-85% confluence, cells were harvested and resuspended in cold PBS with Matrigel at 5×10⁶ cells per milliliter.

Mice were monitored twice weekly following cell implantation for tumor growth. Mice were gently palpated at least twice per week to assess the relative size of the pancreatic tumors. On Day 13, all tumors were assessed, and mice were randomly assigned to treatment groups with 15 mice per group: a control group treated with an IgG control antibody, a group treated with an anti-GITR antibody (described in Example 1) plus gemcitabine (GEM), and a group treated with an anti-GITR antibody, gemcitabine (GEM), and an anti-CSF1R antibody (described in Example 1). The anti-GITR antibody was administered once at 2.5 mg/kg on Day 13; GEM was administered twice weekly at 50 mg/kg beginning on Day 13; and the anti-CSF1R antibody was administered weekly at 30 mg/kg beginning on Day 13. Tumors continued to be assessed at least twice per week for 20 days from the start of treatment.

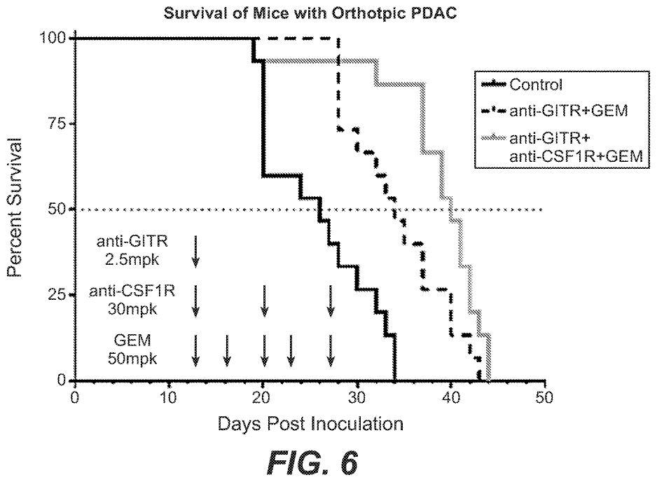

The impact of therapy is shown by graphing animal survival rates throughout the course of the experiment for select groups. (FIG. 6.) Treatment with anti-GITR antibody and GEM significantly increased the survival of PDAC tumor-bearing mice compared to the IgG control (34 days compared to 26 days with p=0.0004). The greatest enhancement of survival rate was observed for animals treated with the combination of anti-GITR, anti-CSF1R, and GEM, with p<0.05 compared to the anti-GITR plus GEM group and p<0.0001 compared to the IgG control group. This group showed a median animal survival of 40 days, with p=0.0275 compared to the anti-GITR/GEM group and p<0.0001 compared to the control group. P-values were calculated using the Log-rank (Mantel-Cox) test comparing individual treatment groups.

### Table of Sequences

The table below provides certain sequences discussed herein. All polypeptide and antibody sequences are shown without leader sequences, unless otherwise indicated.

Sequences and Descriptions

| SEQ ID NO | Description | Sequence |
|---|---|---|
| 1 | hCSF1R | IPVIEPSVPE LVVKPGATVT LRCVGNGSVE WDGPPSPHWT LYSDGSSSIL |
| | (full-length, | STNNATFQNT GTYRCTEPGD PLGGSAAIHL YVKDPARPWN VLAQEVVVFE |
| | no leader | DQDALLPCLL TDPVLEAGVS LVRVRGRPLM RHTNYSFSPW HGFTIHRAKF |
| | sequence) | IQSQDYQCSA LMGGRKVMSI SIRLKVQKVI PGPPALTLVP AELVRIRGEA |
| | | AQIVCSASSV DVNFDVFLQH NNTKLAIPQQ SDFHNNRYQK VLTLNLDQVD |
| | | FQHAGNYSCV ASNVQGKHST SMFFRVVESA YLNLSSEQNL IQEVTVGEGL |
| | | NLKVMVEAYP GLQGFNWTYL GPFSDHQPEP KLANATTKDT YRHTFTLSLP |
| | | RLKPSEAGRY SFLARNPGGW RALTFELTLR YPPEVSVIWT FINGSGTLLC |
| | | AASGYPQPNV TWLQCSGHTD RCDEAQVLQV WDDPYPEVLS QEPFHKVTVQ |
| | | SLLTVETLEH NQTYECRAHN SVGSGSWAFI PISAGAHTHP PDEFLFTPVV |
| | | VACMSIMALL LLLLLLLLYK YKQKPKYQVR WKIIESYEGN SYTFIDPTQL |
| | | PYNEKWEFPR NNLQFGKTLG AGAFGKVVEA TAFGLGKEDA VLKVAVKMLK |
| | | STAHADEKEA LMSELKIMSH LGQHENIVNL LGACTHGGPV LVITEYCCYG |
| | | DLLNFLRRKA EAMLGPSLSP GQDPEGGVDY KNIHLEKKYV RRDSGFSSQG |
| | | VDTYVEMRPV STSSNDSFSE QDLDKEDGRP LELRDLLHFS SQVAQGMAFL |
| | | ASKNCIHRDV AARNVLLTNG HVAKIGDFGL ARDIMNDSNY IVKGNARLPV |
| | | KWMAPESIFD CVYTVQSDVW SYGILLWEIF SLGLNPYPGI LVNSKFYKLV |
| | | KDGYQMAQPA FAPKNIYSIM QACWALEPTH RPTFQQICSF LQEQAQEDRR |
| | | ERDYTNLPSS SRSGGSGSSS SELEEESSSE HLTCCEQGDI AQPLLQPNNY |
| | | QFC |
| 2 | hCSF1R | MGPGVLLLLL VATAWHGQGI PVIEPSVPEL VVKPGATVTL RCVGNGSVEW |
| | | DGPPSPHWTL YSDGSSSILS TNNATFQNTG TYRCTEPGDP LGGSAAIHLY |
| | | VKDPARPWNV LAQEVVVFED QDALLPCLLT DPVLEAGVSL VRVRGRPLMR |
| | | HTNYSFSPWH GFTIHRAKFI QSQDYQCSAL MGGRKVMSIS IRLKVQKVIP |
| | | GPPALTLVPA ELVRIRGEAA QIVCSASSVD VNFDVFLQHN NTKLAIPQQS |
| | | DFHNNRYQKV LTLNLDQVDF QHAGNYSCVA SNVQGKHSTS MFFRVVESAY |
| | | LNLSSEQNLI QEVTVGEGLN LKVMVEAYPG LQGFNWTYLG PFSDHQPEPK |
| | | LANATTKDTY RHTFTLSLPR LKPSEAGRYS FLARNPGGWR ALTFELTLRY |
| | | PPEVSVIWTF INGSGTLLCA ASGYPQPNVT WLQCSGHTDR CDEAQVLQVW |
| | | DDPYPEVLSQ EPFHKVTVQS LLTVETLEHN QTYECRAHNS VGSGSWAFIP |
| | | ISAGAHTHPP DEFLFTPVVV ACMSIMALLL LLLLLLLYKY KQKPKYQVRW |
| | | KIIESYEGNS YTFIDPTQLP YNEKWEFPRN NLQFGKTLGA GAFGKVVEAT |
| | | AFGLGKEDAV LKVAVKMLKS TAHADEKEAL MSELKIMSHL GQHENIVNLL |
| | | GACTHGGPVL VITEYCCYGD LLNFLRRKAE AMLGPSLSPG QDPEGGVDYK |
| | | NIHLEKKYVR RDSGFSSQGV DTYVEMRPVS TSSNDSFSEQ DLDKEDGRPL |
| | | ELRDLLHFSS QVAQGMAFLA SKNCIHRDVA ARNVLLTNGH VAKIGDFGLA |
| | | RDIMNDSNYI VKGNARLPVK WMAPESIFDC VYTVQSDVWS YGILLWEIFS |
| | | LGLNPYPGIL VNSKFYKLVK DGYQMAQPAF APKNIYSIMQ ACWALEPTHR |
| | | PTFQQICSFL QEQAQEDRRE RDYTNLPSSS RSGGSGSSSS ELEEESSSEH |
| | | LTCCEQGDIA QPLLQPNNYQ FC |
| 3 | Light chain | METDTLLLWV LLLWVPGSTG |
| | leader | |
| | sequence | |
| 4 | Heavy chain | MAVLGLLLCL VTFPSCVLS |
| | leader | |
| | sequence | |
| 5 | hCSF1R | IPVIEPSVPE LVVKPGATVT LRCVGNGSVE WDGPPSPHWT LYSDGSSSIL |
| | ECD.506 | STNNATFQNT GTYRCTEPGD PLGGSAAIHL YVKDPARPWN VLAQEVVVFE |
| | | DQDALLPCLL TDPVLEAGVS LVRVRGRPLM RHTNYSFSPW HGFTIHRAKF |
| | | IQSQDYQCSA LMGGRKVMSI SIRLKVQKVI PGPPALTLVP AELVRIRGEA |
| | | AQIVCSASSV DVNFDVFLQH NNTKLAIPQQ SDFHNNRYQK VLTLNLDQVD |
| | | FQHAGNYSCV ASNVQGKHST SMFFRVVESA YLNLSSEQNL IQEVTVGEGL |
| | | NLKVMVEAYP GLQGFNWTYL GPFSDHQPEP KLANATTKDT YRHTFTLSLP |
| | | RLKPSEAGRY SFLARNPGGW RALTFELTLR YPPEVSVIWT FINGSGTLLC |
| | | AASGYPQPNV TWLQCSGHTD RCDEAQVLQV WDDPYPEVLS QEPFHKVTVQ |
| | | SLLTVETLEH NQTYECRAHN SVGSGSWAFI PISAGAH |
| 6 | hCSF1R | IPVIEPSVPE LVVKPGATVT LRCVGNGSVE WDGPPSPHWT LYSDGSSSIL |
| | ECD.506-Fc | STNNATFQNT GTYRCTEPGD PLGGSAAIHL YVKDPARPWN VLAQEVVVFE |
| | | DQDALLPCLL TDPVLEAGVS LVRVRGRPLM RHTNYSFSPW HGFTIHRAKF |
| | | IQSQDYQCSA LMGGRKVMSI SIRLKVQKVI PGPPALTLVP AELVRIRGEA |
| | | AQIVCSASSV DVNFDVFLQH NNTKLAIPQQ SDFHNNRYQK VLTLNLDQVD |
| | | FQHAGNYSCV ASNVQGKHST SMFFRVVESA YLNLSSEQNL IQEVTVGEGL |
| | | NLKVMVEAYP GLQGFNWTYL GPFSDHQPEP KLANATTKDT YRHTFTLSLP |
| | | RLKPSEAGRY SFLARNPGGW RALTFELTLR YPPEVSVIWT FINGSGTLLC |
| | | AASGYPQPNV TWLQCSGHTD RCDEAQVLQV WDDPYPEVLS QEPFHKVTVQ |
| | | SLLTVETLEH NQTYECRAHN SVGSGSWAFI PISAGAHEPK SSDKTHTCPP |
| | | CPAPELLGGP SVFLFPPKPK DTLMISRTPE VTCVVVDVSH EDPEVKFNWY |
| | | VDGVEVHNAK TKPREEQYNS TYRVVSVLTV LHQDWLNGKE YKCKVSNKAL |
| | | PAPIEKTISK AKGQPREPQV YTLPPSRDEL TKNQVSLTCL VKGFYPSDIA |
| | | VEWESNGQPE NNYKTTPPVL DSDGSFFLYS KLTVDKSRWQ QGNVFSCSVM |
| | | HEALHNHYTQ KSLSLSPGK |
| 7 | cynoCSF1R | MGPGVLLLLL VVTAWHGQGI PVIEPSGPEL VVKPGETVTL RCVGNGSVEW |
| | ECD (with | DGPISPHWTL YSDGPSSVLT TTNATFQNTR TYRCTEPGDP LGGSAAIHLY |
| | leader | VKDPARPWNV LAKEVVVFED QDALLPCLLT DPVLEAGVSL VRLRGRPLLR |
| | sequence) | HTNYSFSPWH GFTIHRAKFI QGQDYQCSAL MGSRKVMSIS IRLKVQKVIP |
| | | GPPALTLVPA ELVRIRGEAA QIVCSASNID VDFDVFLQHN TTKLAIPQRS |
| | | DFHDNRYQKV LTLSLGQVDF QHAGNYSCVA SNVQGKHSTS MFFRVVESAY |
| | | LDLSSEQNLI QEVTVGEGLN LKVMVEAYPG LQGFNWTYLG PFSDHQPEPK |
| | | LANATTKDTY RHTFTLSLPR LKPSEAGRYS FLARNPGGWR ALTFELTLRY |
| | | PPEVSVIWTS INGSGTLLCA ASGYPQPNVT WLQCAGHTDR CDEAQVLQVW |
| | | VDPHPEVLSQ EPFQKVTVQS LLTAETLEHN QTYECRAHNS VGSGSWAFIP |
| | | ISAGAR |
| 8 | cynoCSF1R | MGPGVLLLLL VVTAWHGQGI PVIEPSGPEL VVKPGETVTL RCVGNGSVEW |
| | ECD-Fc | DGPISPHWTL YSDGPSSVLT TTNATFQNTR TYRCTEPGDP LGGSAAIHLY |
| | (with leader | VKDPARPWNV LAKEVVVFED QDALLPCLLT DPVLEAGVSL VRLRGRPLLR |
| | sequence) | HTNYSFSPWH GFTIHRAKFI QGQDYQCSAL MGSRKVMSIS IRLKVQKVIP |
| | | GPPALTLVPA ELVRIRGEAA QIVCSASNID VDFDVFLQHN TTKLAIPQRS |
| | | DFHDNRYQKV LTLSLGQVDF QHAGNYSCVA SNVQGKHSTS MFFRVVESAY |
| | | LDLSSEQNLI QEVTVGEGLN LKVMVEAYPG LQGFNWTYLG PFSDHQPEPK |
| | | LANATTKDTY RHTFTLSLPR LKPSEAGRYS FLARNPGGWR ALTFELTLRY |
| | | PPEVSVIWTS INGSGTLLCA ASGYPQPNVT WLQCAGHTDR CDEAQVLQVW |
| | | VDPHPEVLSQ EPFQKVTVQS LLTAETLEHN QTYECRAHNS VGSGSWAFIP |
| | | ISAGARGSEP KSSDKTHTCP PCPAPELLGG PSVFLFPPKP KDTLMISRTP |
| | | EVTCVVVDVS HEDPEVKFNW YVDGVEVHNA KTKPREEQYN STYRVVSVLT |
| | | VLHQDWLNGK EYKCKVSNKA LPAPIEKTIS KAKGQPREPQ VYTLPPSRDE |
| | | LTKNQVSLTC LVKGFYPSDI AVEWESNGQP ENNYKTTPPV LDSDGSFFLY |
| | | SKLTVDKSRW QQGNVFSCSV MHEALHNHYT QKSLSLSPGK |
| 9 | Fab 0301 | EVQLQQSGPE LVRPGASVKM SCKASGYTFT DNYMIWVKQS HGKSLEWIGD |
| | heavy chain | INPYNGGTTF NQKFKGKATL TVEKSSSTAY MQLNSLTSED SAVYYCARES |
| | variable | PYFSNLYVMD YWGQGTSVTV SS |
| | region | |
| 10 | Fab 0301 | NIVLTQSPAS LAVSLGQRAT ISCKASQSVD YDGDNYMNWY QQKPGQPPKL |
| | light chain | LIYAASNLES GIPARFSGSG SGTDFTLNIH PVEEEDAATY YCHLSNEDLS |
| | variable | TFGGGTKLEI K |
| | region | |
| 11 | Fab 0302 | EIQLQQSGPE LVKPGASVKM SCKASGYTFS DFNIHWVKQK PGQGLEWIGY |
| | heavy chain | INPYTDVTVY NEKFKGKATL TSDRSSSTAY MDLSSLTSED SAVYYCASYF |
| | variable | DGTFDYALDY WGQGTSITVS S |
| | region | |
| 12 | Fab 0302 | DVVVTQTPAS LAVSLGQRAT ISCRASESVD NYGLSFMNWF QQKPGQPPKL |
| | light chain | LIYTASNLES GIPARFSGGG SRTDFTLTID PVEADDAATY FCQQSKELPW |
| | variable | TFGGGTRLEI K |
| | region | |
| 13 | Fab 0311 | EIQLQQSGPD LMKPGASVKM SCKASGYIFT DYNMHWVKQN QGKSLEWMGE |
| | heavy chain | INPNNGVVVY NQKFKGTTTL TVDKSSSTAY MDLHSLTSED SAVYYCTRAL |
| | variable | YHSNFGWYFD SWGKGTTLTV SS |
| | region | |
| 14 | Fab 0311 | DIVLTQSPAS LAVSLGQRAT ISCKASQSVD YDGDSHMNWY QQKPGQPPKL |
| | light chain | LIYTASNLES GIPARFSGSG SGADFTLTIH PVEEEDAATY YCQQGNEDPW |
| | variable | TFGGGTRLEI K |
| | region | |
| 15 | 0301 heavy | GYTFTDNYMI |
| | chain CDR1 | |
| 16 | 0301 heavy | DINPYNGGTT FNQKFKG |
| | chain CDR2 | |
| 17 | 0301 heavy | ESPYFSNLYV MDY |
| | chain CDR3 | |
| 18 | 0301 light | KASQSVDYDG DNYMN |
| | chain CDR1 | |
| 19 | 0301 light | AASNLES |
| | chain CDR2 | |
| 20 | 0301 light | HLSNEDLST |
| | chain CDR3 | |
| 21 | 0302 heavy | GYTFSDFNIH |
| | chain CDR1 | |
| 22 | 0302 heavy | YINPYTDVTV YNEKFKG |
| | chain CDR2 | |
| 23 | 0302 heavy | YFDGTFDYAL DY |
| | chain CDR3 | |
| 24 | 0302 light | RASESVDNYG LSFMN |
| | chain CDR1 | |
| 25 | 0302 light | TASNLES |
| | chain CDR2 | |
| 26 | 0302 light | QQSKELPWT |
| | chain CDR3 | |
| 27 | 0311 heavy | GYIFTDYNMH |
| | chain CDR1 | |
| 28 | 0311 heavy | EINPNNGVVV YNQKFKG |
| | chain CDP2 | |
| 29 | 0311 heavy | ALYHSNFGWY FDS |
| | chain CDR3 | |
| 30 | 0311 light | KASQSVDYDG DSHMN |
| | chain CDR1 | |
| 31 | 0311 light | TASNLES |
| | chain CDR2 | |
| 32 | 0311 light | QQGNEDPWT |
| | chain CDR3 | |
| 33 | cAb 0301 | EVQLQQSGPE LVRPGASVKM SCKASGYTFT DNYMIWVKQS HGKSLEWIGD |
| | heavy chain | INPYNGGTTF NQKFKGKATL TVEKSSSTAY MQLNSLTSED SAVYYCARES |
| | | PYFSNLYVMD YWGQGTSVTV SSASTKGPSV FPLAPCSRST SESTAALGCL |
| | | VKDYFPEPVT VSWNSGALTS GVHTFPAVLQ SSGLYSLSSV VTVPSSSLGT |
| | | KTYTCNVDHK PSNTKVDKRV ESKYGPPCPP CPAPEFLGGP SVFLFPPKPK |
| | | DTLMISRTPE VTCVVVDVSQ EDPEVQFNWY VDGVEVHNAK TKPREEQFNS |
| | | TYRVVSVLTV LHQDWLNGKE YKCKVSNKGL PSSIEKTISK AKGQPREPQV |
| | | YTLPPSQEEM TKNQVSLTCL VKGFYPSDIA VEWESNGQPE NNYKTTPPVL |
| | | DSDGSFFLYS RLTVDKSRWQ EGNVFSCSVM HEALHNHYTQ KSLSLSLGK |
| 34 | cAb 0301 | NIVLTQSPAS LAVSLGQRAT ISCKASQSVD YDGDNYMNWY QQKPGQPPKL |
| | light chain | LIYAASNLES GIPARFSGSG SGTDFTLNIH PVEEEDAATY YCHLSNEDLS |
| | | TFGGGTKLEI KRTVAAPSVF IFPPSDEQLK SGTASVVCLL NNFYPREAKV |
| | | QWKVDNALQS GNSQESVTEQ DSKDSTYSLS STLTLSKADY EKHKVYACEV |
| | | THQGLSSPVT KSFNRGEC |
| 35 | cAb 0302 | EIQLQQSGPE LVKPGASVKM SCKASGYTFS DFNIHWVKQK PGQGLEWIGY |
| | heavy chain | INPYTDVTVY NEKFKGKATL TSDRSSSTAY MDLSSLTSED SAVYYCASYF |
| | | DGTFDYALDY WGQGTSITVS SASTKGPSVF PLAPCSRSTS ESTAALGCLV |
| | | KDYFPEPVTV SWNSGALTSG VHTFPAVLQS SGLYSLSSVV TVPSSSLGTK |
| | | TYTCNVDHKP SNTKVDKRVE SKYGPPCPPC PAPEFLGGPS VFLFPPKPKD |
| | | TLMISRTPEV TCVVVDVSQE DPEVQFNWYV DGVEVHNAKT KPREEQFNST |
| | | YRVVSVLTVL HQDWLNGKEY KCKVSNKGLP SSIEKTISKA KGQPREPQVY |
| | | TLPPSQEEMT KNQVSLTCLV KGFYPSDIAV EWESNGQPEN NYKTTPPVLD |
| | | SDGSFFLYSR LTVDKSRWQE GNVFSCSVMH EALHNHYTQK SLSLSLGK |
| 36 | cAb 0302 | DVVVTQTPAS LAVSLGQRAT ISCRASESVD NYGLSFMNWF QQKPGQPPKL |
| | light chain | LIYTASNLES GIPARFSGGG SRTDFTLTID PVEADDAATY FCQQSKELPW |
| | | TFGGGTRLEI KRTVAAPSVF IFPPSDEQLK SGTASVVCLL NNFYPREAKV |
| | | QWKVDNALQS GNSQESVTEQ DSKDSTYSLS STLTLSKADY EKHKVYACEV |
| | | THQGLSSPVT KSFNRGEC |
| 37 | cAb 0311 | EIQLQQSGPD LMKPGASVKM SCKASGYIFT DYNMHWVKQN QGKSLEWMGE |
| | heavy chain | INPNNGVVVY NQKFKGTTTL TVDKSSSTAY MDLHSLTSED SAVYYCTRAL |
| | | YHSNFGWYFD SWGKGTTLTV SSASTKGPSV FPLAPCSRST SESTAALGCL |
| | | VKDYFPEPVT VSWNSGALTS GVHTFPAVLQ SSGLYSLSSV VTVPSSSLGT |
| | | KTYTCNVDHK PSNTKVDKRV ESKYGPPCPP CPAPEFLGGP SVFLFPPKPK |
| | | DTLMISRTPE VTCVVVDVSQ EDPEVQFNWY VDGVEVHNAK TKPREEQFNS |
| | | TYRVVSVLTV LHQDWLNGKE YKCKVSNKGL PSSIEKTISK AKGQPREPQV |
| | | YTLPPSQEEM TKNQVSLTCL VKGFYPSDIA VEWESNGQPE NNYKTTPPVL |
| | | DSDGSFFLYS RLTVDKSRWQ EGNVFSCSVM HEALHNHYTQ KSLSLSLGK |
| 38 | cAb 0311 | DIVLTQSPAS LAVSLGQRAT ISCKASQSVD YDGDSHMNWY QQKPGQPPKL |
| | light chain | LIYTASNLES GIPARFSGSG SGADFTLTIH PVEEEDAATY YCQQGNEDPW |
| | | TFGGGTRLEI KRTVAAPSVF IFPPSDEQLK SGTASVVCLL NNFYPREAKV |
| | | QWKVDNALQS GNSQESVTEQ DSKDSTYSLS STLTLSKADY EKHKVYACEV |
| | | THQGLSSPVT KSFNRGEC |
| 39 | h0301-H0 | QVQLVQSGAE VKKPGSSVKV SCKASGYTFT DNYMIWVRQA PGQGLEWMGD |
| | heavy chain | INPYNGGTTF NQKFKGRVTI TADKSTSTAY MELSSLRSED TAVYYCARES |
| | variable | PYFSNLYVMD YWGQGTLVTV SS |
| 40 | h0301-H1 | QVQLVQSGAE VKKPGSSVKV SCKASGYTFT DNYMIWVRQA PGQGLEWMGD |
| | heavy chain | NQKFKGRVTI TVDKSTSTAY MELSSLRSED TAVYYCARES |
| | variable region | PYFSNLYVMD YWGQGTLVTV SS |
| 41 | h0301-H2 | QVQLVQSGAE VKKPGSSVKV SCKASGYTFT DNYMIWVRQA PGQGLEWIGD |
| | heavy chain | INPYNGGTTF NQKFKGRATL TVDKSTSTAY MELSSLRSED TAVYYCARES |
| | variable region | PYFSNLYVMD YWGQGTLVTV SS |
| 42 | H0302-H1 | QVQLVQSGAE VKKPGSSVKV SCKASGYTFS DFNIHWVRQA PGQGLEWMGY |
| | heavy chain | INPYTDVTVY NEKFKGRVTI TSDKSTSTAY MELSSLRSED TAVYYCASYF |
| | variable region | DGTFDYALDY WGQGTLVTVS S |
| 43 | H0302-H2 | QVQLVQSGAE VKKPGSSVKV SCKASGYTFS DFNIHWVRQA PGQGLEWIGY |
| | heavy chain | INPYTDVTVY NEKFKGRATL TSDKSTSTAY MELSSLRSED TAVYYCASYF |
| | variable region | DGTFDYALDY WGQGTLVTVS S |
| 44 | H0311-H1 | QVQLVQSGAE VKKPGSSVKV SCKASGYIFT DYNMHWVRQA PGQGLEWMGE |
| | heavy chain | INPNNGVVVY NQKFKGRVTI TVDKSTSTAY MELSSLRSED TAVYYCTRAL |
| | variable region | YHSNFGWYFD SWGQGTLVTV SS |
| 45 | H0311-H2 | QVQLVQSGAE VKKPGSSVKV SCKASGYIFT DYNMHWVRQA PGQGLEWMGE |
| | heavy chain | INPNNGVVVY NQKFKGTTTL TVDKSTSTAY MELSSLRSED TAVYYCTRAL |
| | variable region | YHSNFGWYFD SWGQGTLVTV SS |
| 46 | h0301-L0 | EIVLTQSPAT LSLSPGERAT LSCKASQSVD YDGDNYMNWY QQKPGQAPRL |
| | light chain | LIYAASNLES GIPARFSGSG SGTDFTLTIS SLEPEDFAVY YCHLSNEDLS |
| | variable region | TFGGGTKVEI K |
| 47 | h0301-L1 | NIVLTQSPAT LSLSPGERAT LSCKASQSVD YDGDNYMNWY QQKPGQAPRL |
| | light chain | LIYAASNLES GIPARFSGSG SGTDFTLTIS SLEPEDFAVY YCHLSNEDLS |
| | variable region | TFGGGTKVEI K |
| 48 | H0302-L0 | EIVLTQSPAT LSLSPGERAT LSCRASESVD NYGLSFMNWY QQKPGQAPRL |
| | light chain | LIYTASNLES GIPARFSGSG SGTDFTLTIS SLEPEDFAVY YCQQSKELPW |
| | variable region | TFGQGTKVEI K |
| 49 | H0302-L1 | EIVLTQSPAT LSLSPGERAT LSCRASESVD NYGLSFMNWY QQKPGQAPRL |
| | light chain | LIYTASNLES GIPARFSGSG SRTDFTLTIS SLEPEDFAVY YCQQSKELPW |
| | variable region | TFGQGTKVEI K |
| 50 | H0302-L2 | EIVVTQSPAT LSLSPGERAT LSCRASESVD NYGLSFMNWF QQKPGQAPRL |
| | light chain | LIYTASNLES GIPARFSGSG SRTDFTLTIS SLEPEDFAVY YCQQSKELPW |
| | variable region | TFGQGTKVEI K |
| 51 | H0311-L0 | EIVLTQSPAT LSLSPGERAT LSCKASQSVD YDGDSHMNWY QQKPGQAPRL |
| | light chain | LIYTASNLES GIPARFSGSG SGTDFTLTIS SLEPEDFAVY YCQQGNEDPW |
| | variable region | TFGQGTKVEI K |
| 52 | H0311-L1 | DIVLTQSPAT LSLSPGERAT LSCKASQSVD YDGDSHMNWY QQKPGQAPRL |
| | light chain | LIYTASNLES GIPARFSGSG SGADFTLTIS SLEPEDFAVY YCQQGNEDPW |
| | variable region | TFGQGTKVEI K |
| 53 | h0301-H0 | QVQLVQSGAE VKKPGSSVKV SCKASGYTFT DNYMIWVRQA PGQGLEWMGD |
| | heavy chain | INPYNGGTTF NQKFKGRVTI TADKSTSTAY MELSSLRSED TAVYYCARES |
| | | PYFSNLYVMD YWGQGTLVTV SSASTKGPSV FPLAPCSRST SESTAALGCL |
| | | VKDYFPEPVT VSWNSGALTS GVHTFPAVLQ SSGLYSLSSV VTVPSSSLGT |
| | | KTYTCNVDHK PSNTKVDKRV ESKYGPPCPP CPAPEFLGGP SVFLFPPKPK |
| | | DTLMISRTPE VTCVVVDVSQ EDPEVQFNWY VDGVEVHNAK TKPREEQFNS |
| | | TYRVVSVLTV LHQDWLNGKE YKCKVSNKGL PSSIEKTISK AKGQPREPQV |
| | | YTLPPSQEEM TKNQVSLTCL VKGFYPSDIA VEWESNGQPE NNYKTTPPVL |
| | | DSDGSFFLYS RLTVDKSRWQ EGNVFSCSVM HEALHNHYTQ KSLSLSLGK |
| 54 | h0301-H1 | QVQLVQSGAE VKKPGSSVKV SCKASGYTFT DNYMIWVRQA PGQGLEWMGD |
| | heavy chain | INPYNGGTTF NQKFKGRVTI TVDKSTSTAY MELSSLRSED TAVYYCARES |
| | | PYFSNLYVMD YWGQGTLVTV SSASTKGPSV FPLAPCSRST SESTAALGCL |
| | | VKDYFPEPVT VSWNSGALTS GVHTFPAVLQ SSGLYSLSSV VTVPSSSLGT |
| | | KTYTCNVDHK PSNTKVDKRV ESKYGPPCPP CPAPEFLGGP SVFLFPPKPK |
| | | DTLMISRTPE VTCVVVDVSQ EDPEVQFNWY VDGVEVHNAK TKPREEQFNS |
| | | TYRVVSVLTV LHQDWLNGKE YKCKVSNKGL PSSIEKTISK AKGQPREPQV |
| | | YTLPPSQEEM TKNQVSLTCL VKGFYPSDIA VEWESNGQPE NNYKTTPPVL |
| | | DSDGSFFLYS RLTVDKSRWQ EGNVFSCSVM HEALHNHYTQ KSLSLSLGK |
| 55 | h0301-H2 | QVQLVQSGAE VKKPGSSVKV SCKASGYTFT DNYMIWVRQA PGQGLEWIGD |
| | heavy chain | INPYNGGTTF NQKFKGRATL TVDKSTSTAY MELSSLRSED TAVYYCARES |
| | | PYFSNLYVMD YWGQGTLVTV SSASTKGPSV FPLAPCSRST SESTAALGCL |
| | | VKDYFPEPVT VSWNSGALTS GVHTFPAVLQ SSGLYSLSSV VTVPSSSLGT |
| | | KTYTCNVDHK PSNTKVDKRV ESKYGPPCPP CPAPEFLGGP SVFLFPPKPK |
| | | DTLMISRTPE VTCVVVDVSQ EDPEVQFNWY VDGVEVHNAK TKPREEQFNS |
| | | TYRVVSVLTV LHQDWLNGKE YKCKVSNKGL PSSIEKTISK AKGQPREPQV |
| | | YTLPPSQEEM TKNQVSLTCL VKGFYPSDIA VEWESNGQPE NNYKTTPPVL |
| | | DSDGSFFLYS RLTVDKSRWQ EGNVFSCSVM HEALHNHYTQ KSLSLSLGK |
| 56 | H0302-H1 | QVQLVQSGAE VKKPGSSVKV SCKASGYTFS DFNIHWVRQA PGQGLEWMGY |
| | heavy chain | INPYTDVTVY NEKFKGRVTI TSDKSTSTAY MELSSLRSED TAVYYCASYF |
| | | DGTFDYALDY WGQGTLVTVS SASTKGPSVF PLAPCSRSTS ESTAALGCLV |
| | | KDYFPEPVTV SWNSGALTSG VHTFPAVLQS SGLYSLSSVV TVPSSSLGTK |
| | | TYTCNVDHKP SNTKVDKRVE SKYGPPCPPC PAPEFLGGPS VFLFPPKPKD |
| | | TLMISRTPEV TCVVVDVSQE DPEVQFNWYV DGVEVHNAKT KPREEQFNST |
| | | YRVVSVLTVL HQDWLNGKEY KCKVSNKGLP SSIEKTISKA KGQPREPQVY |
| | | TLPPSQEEMT KNQVSLTCLV KGFYPSDIAV EWESNGQPEN NYKTTPPVLD |
| | | SDGSFFLYSR LTVDKSRWQE GNVFSCSVMH EALHNHYTQK SLSLSLGK |
| 57 | H0302-H2 | QVQLVQSGAE VKKPGSSVKV SCKASGYTFS DFNIHWVRQA PGQGLEWIGY |
| | heavy chain | INPYTDVTVY NEKFKGRATL TSDKSTSTAY MELSSLRSED TAVYYCASYF |
| | | DGTFDYALDY WGQGTLVTVS SASTKGPSVF PLAPCSRSTS ESTAALGCLV |
| | | KDYFPEPVTV SWNSGALTSG VHTFPAVLQS SGLYSLSSVV TVPSSSLGTK |
| | | TYTCNVDHKP SNTKVDKRVE SKYGPPCPPC PAPEFLGGPS VFLFPPKPKD |
| | | TLMISRTPEV TCVVVDVSQE DPEVQFNWYV DGVEVHNAKT KPREEQFNST |
| | | YRVVSVLTVL HQDWLNGKEY KCKVSNKGLP SSIEKTISKA KGQPREPQVY |
| | | TLPPSQEEMT KNQVSLTCLV KGFYPSDIAV EWESNGQPEN NYKTTPPVLD |
| | | SDGSFFLYSR LTVDKSRWQE GNVFSCSVMH EALHNHYTQK SLSLSLGK |
| 58 | H0311-H1 | QVQLVQSGAE VKKPGSSVKV SCKASGYIFT DYNMHWVRQA PGQGLEWMGE |
| | heavy chain | INPNNGVVVY NQKFKGRVTI TVDKSTSTAY MELSSLRSED TAVYYCTRAL |
| | | YHSNFGWYFD SWGQGTLVTV SSASTKGPSV FPLAPCSRST SESTAALGCL |
| | | VKDYFPEPVT VSWNSGALTS GVHTFPAVLQ SSGLYSLSSV VTVPSSSLGT |
| | | KTYTCNVDHK PSNTKVDKRV ESKYGPPCPP CPAPEFLGGP SVFLFPPKPK |
| | | DTLMISRTPE VTCVVVDVSQ EDPEVQFNWY VDGVEVHNAK TKPREEQFNS |
| | | TYRVVSVLTV LHQDWLNGKE YKCKVSNKGL PSSIEKTISK AKGQPREPQV |
| | | YTLPPSQEEM TKNQVSLTCL VKGFYPSDIA VEWESNGQPE NNYKTTPPVL |
| | | DSDGSFFLYS RLTVDKSRWQ EGNVFSCSVM HEALHNHYTQ KSLSLSLGK |
| 59 | H0311-H2 | QVQLVQSGAE VKKPGSSVKV SCKASGYIFT DYNMHWVRQA PGQGLEWMGE |
| | heavy chain | INPNNGVVVY NQKFKGTTTL TVDKSTSTAY MELSSLRSED TAVYYCTRAL |
| | | YHSNFGWYFD SWGQGTLVTV SSASTKGPSV FPLAPCSRST SESTAALGCL |
| | | VKDYFPEPVT VSWNSGALTS GVHTFPAVLQ SSGLYSLSSV VTVPSSSLGT |
| | | KTYTCNVDHK PSNTKVDKRV ESKYGPPCPP CPAPEFLGGP SVFLFPPKPK |
| | | DTLMISRTPE VTCVVVDVSQ EDPEVQFNWY VDGVEVHNAK TKPREEQFNS |
| | | TYRVVSVLTV LHQDWLNGKE YKCKVSNKGL PSSIEKTISK AKGQPREPQV |
| | | YTLPPSQEEM TKNQVSLTCL VKGFYPSDIA VEWESNGQPE NNYKTTPPVL |
| | | DSDGSFFLYS RLTVDKSRWQ EGNVFSCSVM HEALHNHYTQ KSLSLSLGK |
| 60 | h0301-L0 | EIVLTQSPAT LSLSPGERAT LSCKASQSVD YDGDNYMNWY QQKPGQAPRL |
| | light chain | LIYAASNLES GIPARFSGSG SGTDFTLTIS SLEPEDFAVY YCHLSNEDLS |
| | | TFGGGTKVEI KRTVAAPSVF IFPPSDEQLK SGTASVVCLL NNFYPREAKV |
| | | QWKVDNALQS GNSQESVTEQ DSKDSTYSLS STLTLSKADY EKHKVYACEV |
| | | THQGLSSPVT KSFNRGEC |
| 61 | h0301-L1 | NIVLTQSPAT LSLSPGERAT LSCKASQSVD YDGDNYMNWY QQKPGQAPRL |
| | light chain | LIYAASNLES GIPARFSGSG SGTDFTLTIS SLEPEDFAVY YCHLSNEDLS |
| | | TFGGGTKVEI KRTVAAPSVF IFPPSDEQLK SGTASVVCLL NNFYPREAKV |
| | | QWKVDNALQS GNSQESVTEQ DSKDSTYSLS STLTLSKADY EKHKVYACEV |
| | | THQGLSSPVT KSFNRGEC |
| 62 | H0302-L0 | EIVLTQSPAT LSLSPGERAT LSCRASESVD NYGLSFMNWY QQKPGQAPRL |
| | light chain | LIYTASNLES GIPARFSGSG SGTDFTLTIS SLEPEDFAVY YCQQSKELPW |
| | | TFGQGTKVEI KRTVAAPSVF IFPPSDEQLK SGTASVVCLL NNFYPREAKV |
| | | QWKVDNALQS GNSQESVTEQ DSKDSTYSLS STLTLSKADY EKHKVYACEV |
| | | THQGLSSPVT KSFNRGEC |
| 63 | H0302-L1 | EIVLTQSPAT LSLSPGERAT LSCRASESVD NYGLSFMNWY QQKPGQAPRL |
| | light chain | LIYTASNLES GIPARFSGSG SRTDFTLTIS SLEPEDFAVY YCQQSKELPW |
| | | TFGQGTKVEI KRTVAAPSVF IFPPSDEQLK SGTASVVCLL NNFYPREAKV |
| | | QWKVDNALQS GNSQESVTEQ DSKDSTYSLS STLTLSKADY EKHKVYACEV |
| | | THQGLSSPVT KSFNRGEC |
| 64 | H0302-L2 | EIVVTQSPAT LSLSPGERAT LSCRASESVD NYGLSFMNWF QQKPGQAPRL |
| | light chain | LIYTASNLES GIPARFSGSG SRTDFTLTIS SLEPEDFAVY YCQQSKELPW |
| | | TFGQGTKVEI KRTVAAPSVF IFPPSDEQLK SGTASVVCLL NNFYPREAKV |
| | | QWKVDNALQS GNSQESVTEQ DSKDSTYSLS STLTLSKADY EKHKVYACEV |
| | | THQGLSSPVT KSFNRGEC |
| 65 | H0311-L0 | EIVLTQSPAT LSLSPGERAT LSCKASQSVD YDGDSHMNWY QQKPGQAPRL |
| | light chain | LIYTASNLES GIPARFSGSG SGTDFTLTIS SLEPEDFAVY YCQQGNEDPW |
| | | TFGQGTKVEI KRTVAAPSVF IFPPSDEQLK SGTASVVCLL NNFYPREAKV |
| | | QWKVDNALQS GNSQESVTEQ DSKDSTYSLS STLTLSKADY EKHKVYACEV |
| | | THQGLSSPVT KSFNRGEC |
| 66 | H0311-L1 | DIVLTQSPAT LSLSPGERAT LSCKASQSVD YDGDSHMNWY QQKPGQAPRL |
| | light chain | LIYTASNLES GIPARFSGSG SGADFTLTIS SLEPEDFAVY YCQQGNEDPW |
| | | TFGQGTKVEI KRTVAAPSVF IFPPSDEQLK SGTASVVCLL NNFYPREAKV |
| | | QWKVDNALQS GNSQESVTEQ DSKDSTYSLS STLTLSKADY EKHKVYACEV |
| | | THQGLSSPVT KSFNRGEC |
| 67 | Human CSF1 | EEVSEYCSHM IGSGHLQSLQ RLIDSQMETS CQITFEFVDQ EQLKDPVCYL |
| | | KKAFLLVQDI MEDTMRFRDN TPNAIAIVQL QELSLRLKSC FTKDYEEHDK |
| | | ACVRTFYETP LQLLEKVKNV FNETKNLLDK DWNIFSKNCN NSFAECSSQG |
| | | HERQSEGS |
| 68 | Human IL-34 | NEPLEMWPLT QNEECTVTGF LRDKLQYRSR LQYMKHYFPI NYKISVPYEG |
| | | VFRIANVTRL QRAQVSEREL RYLWVLVSLSATESVQDVLL EGHPSWKYLQ |
| | | EVQTLLLNVQ QGLTDVEVSP KVESVLSLLN APGPNLKLVR PKALLDNCFR |
| | | VMELLYCSCC KQSSVLNWQD CEVPSPQSCS PEPSLQYAAT QLYPPPPWSP |
| | | SSPPHSTGSV RPVRAQGEGL LP |
| 69 | Human | QVQLVQSGAE VKKPGSSVKV SCKAS |
| | acceptor A | |
| | FR1 | |
| 70 | Human | WVRQAPGQGL EWMG |
| | acceptor A | |
| | FR2 | |
| 71 | Human | EVTITADKST STAYMELSSL RSEDTAVYYC AR |
| | acceptor A | |
| | FR3 | |
| 72 | Human | WGQGTLVTVS S |
| | acceptor A | |
| | FR4 | |
| 73 | Human | QVQLVQSGAE VKKPGSSVKV SCKAS |
| | acceptor B | |
| | FR1 | |
| 74 | Human | WVRQAPGQGL EWMG |
| | acceptor B | |
| | FR2 | |
| 75 | Human | RVTITADKST STAYMELSSL RSEDTAVYYC AR |
| | acceptor B | |
| | FR3 | |
| 76 | Human | WGQGTLVTVSS |
| | acceptor B | |
| | FR4 | |
| 77 | Human | QVQLVQSGAE VKKPGSSVKV SCKAS |
| | acceptor C | |
| | FR1 | |
| 78 | Human | WVRQAPGQGL EWMG |
| | acceptor C | |
| | FR2 | |
| 79 | Human | RVTITADKST STAYMELSSL RSEDTAVYYC AR |
| | acceptor C | |
| | FR3 | |
| 80 | Human | WGQGTLVTVS S |
| | acceptor C | |
| | FR2 | |
| 81 | Human | EIVLTQSPAT LSLSPGERAT LSC |
| | acceptor C | |
| | FR2 | |
| 82 | Human | WYQQKPGQAP RLLIY |
| | acceptor D | |
| | FR2 | |
| 83 | Human | GIPARFSGSG SGTDFTLTIS SLEPEDFAVY YC |
| | acceptor D | |
| | FR3 | |
| 84 | Human | FGGGTKVEIK |
| | acceptor 3 | |
| | FR4 | |
| 85 | Human | EIVLTQSPAT LSLSPGERAT LSC |
| | acceptor E | |
| | FR1 | |
| 86 | Human | WYQQKPGQAP RLLIY |
| | acceptor E | |
| | FR2 | |
| 87 | Human | GIPARFSGSG SGTDFTLTIS SLEPEDFAVY YC |
| | acceptor E | |
| | FR3 | |
| 88 | Human | FGQGTKVEIK |
| | acceptor E | |
| | FR4 | |
| 89 | Human | EIVLTQSPAT LSLSPGERAT LSC |
| | acceptor F | |
| | FR1 | |
| 90 | Human | WYQQKPGQAP RLLIY |
| | acceptor F | |
| | FR2 | |
| 91 | Human | GIPARFSGSG SGTDFTLTIS SLEPEDFACY YC |
| | acceptor F | |
| | FR3 | |
| 92 | Human | FGQGTKVEIK |
| | acceptor F | |
| | FR4 | |
| 93 | mCSF1R | APVIEPSGPE LVVEPGETVT LRCVSNGSVE WDGPISPYWT LDPESPGSTL |
| | ECD-Fc | TTRNATFKNT GTYRCTELED PMAGSTTIHL YVKDPAHSWN LLAQEVTVVE |
| | | GQEAVLPCLI TDPALKDSVS LMREGGRQVL RKTVYFFSPW RGFIIRKAKV |
| | | LDSNTYVCKT MVNGRESTST GIWLKVNRVH PEPPQIKLEP SKLVRIRGEA |
| | | AQIVCSATNA EVGFNVILKR GDTKLEIPLN SDFQDNYYKK VRALSLNAVD |
| | | FQDAGIYSCV ASNDVGTRTA TMNFQVVESA YLNLTSEQSL LQEVSVGDSL |
| | | ILTVHADAYP SIQHYNWTYL GPFFEDQRKL EFITQRAIYR YTFKLFLNRV |
| | | KASEAGQYFL MAQNKAGWNN LTFELTLRYP PEVSVTWMPV NGSDVLFCDV |
| | | SGYPQPSVTW MECRGHTDRC DEAQALQVWN DTHPEVLSQK PFDKVIIQSQ |
| | | LPIGTLKHNM TYFCKTHNSV GNSSQYFRAV SLGQSKQEPK SSDKTHTCPP |
| | | CPAPELLGGP SVFLFPPKPK DTLMISRTPE VTCVVVDVSH EDPEVKFNWY |
| | | VDGVEVHNAK TKPREEQYNS TYRVVSVLTV LHQDWLNGKE YKCKVSNKAL |
| | | PAPIEKTISK AKGQPREPQV YTLPPSRDEL TKNQVSLTCL VKGFYPSDIA |
| | | VEWESNGQPE NNYKTTPPVL DSDGSFFLYS KLTVDKSRWQ QGNVFSCSVM |
| | | HEALHNHYTQ KSLSLSPGK |
| 94 | Human | ASTKGPSVFP LAPCSRSTSE STAALGCLVK DYFPEPVTVS WNSGALTSGV |
| | IgG4 S241P | HTFPAVLQSS GLYSLSSVVT VPSSSLGTKT YTCNVDHKPS NTKVDKRVES |
| | | KYGPPCPPCP APEFLGGPSV FLFPPKPKDT LMISRTPEVT CVVVDVSQED |
| | | PEVQFNWYVD GVEVHNAKTK PREEQFNSTY RVVSVLTVLH QDWLNGKEYK |
| | | CKVSNKGLPS SIEKTISKAK GQPREPQVYT LPPSQEEMTK NQVSLTCLVK |
| | | GFYPSDIAVE WESNGQPENN YKTTPPVLDS DGSFFLYSRL TVDKSRWQEG |
| | | NVFSCSVMHE ALHNHYTQKS LSLSLGK |
| 95 | Human Igκ | RTVAAPSVFI FPPSDEQLKS GTASVVCLLN NFYPREAKVQ WKVDNALQSG |
| | | NSQESVTEQD SKDSTYSLSS TLTLSKADYE KHKVYACEVT HQGLSSPVTK |
| | | SFNRGEC |
| 96 | human PD-1 | MQIPQAPWPV VWAVLQLGWR PGWFLDSPDR PWNPPTFSPA LLVVTEGDNA |
| | precursor | TFTCSFSNTS ESFVLNWYRM SPSNQTDKLA AFPEDRSQPG QDCRFRVTQL |
| | (with signal | PNGRDFHMSV VRARRNDSGT YLCGAISLAP KAQIKESLRA ELRVTERRAE |
| | sequence) | VPTAHPSPSP RPAGQFQTLV VGVVGGLLGS LVLLVWVLAV ICSRAARGTI |
| | UniProtKB/ | GARRTGQPLK EDPSAVPVFS VDYGELDFQW REKTPEPPVP CVPQETEYAT |
| | Swiss-Prot: | IVFPSGMGTS SPARRGSADG PRSAQPLRPE DGHCSWPL |
| | Q15116.3, | |
| | Oct. 01, 2014 | |
| 97 | human PD-1 | PGWFLDSPDR PWNPPTFSPA LLVVTEGDNA TFTCSFSNTS ESFVLNWYRM |
| | (mature, | SPSNQTDKLA AFPEDRSQPG QDCRFRVTQL PNGRDFHMSV VRARRNDSGT |
| | without signal | YLCGAISLAP KAQIKESLRA ELRVTERRAE VPTAHPSPSP RPAGQFQTLV |
| | sequence) | VGVVGGLLGS LVLLVWVLAV ICSRAARGTI GARRTGQPLK EDPSAVPVFS |
| | | VDYGELDFQW REKTPEPPVP CVPEQTEYAT IVFPSGMGTS SPARRGSADG |
| | | PRSAQPLRPE DGHCSWPL |
| 98 | humna PD-L1 | MRIFAVFIFM TYWHLLNAFT VTVPKDLYVV EYGSNMTIEC KFPVEKQLDL |
| | precursor | AALIVYWEME DKNIIQFVHG EEDLKVQHSS YRQRARLLKD QLSLGNAALQ |
| | (with signal | ITDVKLQDAG VYRCMISYGG ADYKRITVKV NAPYNKINQR ILVVDPVTSE |
| | sequence) | HELTCQAEGY PKAEVIWTSS DHQVLSGKTT TTNSKREEKL FNVTSTLRIN |
| | UniProtKB/ | TTTNEIFYCT FRRLDPEENH TAELVIPELP LAHPPNERTH LVILGAILLC |
| | Swiss-Prot: | LGVALTFIFR LRKGRMMDVK KCGIQDTNSK KQSDTHLEET |
| | Q9NZQ7.1, | |
| | Oct. 01, 2014 | |
| 99 | human PD-L1 | FT VTVPKDLYVV EYGSNMTIEC KFPVEKQLDL AALIVYWEME |
| | (mature, | DKNIIQFVHG EEDLKVQHSS YRQRARLLKD QLSLGNAALQ ITDVKLQDAG |
| | without signal | VYRCMISYGG ADYKRITVKV NAPYNKINQR ILVVDPVTSE HELTCQAEGY |
| | sequence) | PKAEVIWTSS DHQVLSGKTT TTNSKREEKL FNVTSTLRIN TTTNEIFYCT |
| | | FRRLDPEENH TAELVIPELP LHAPPNERTH LVILGAILLC LGVALTFIFR |
| | | LRKGRMMDVK KCGIQDTNSK KQSDTHLEET |
| 100 | Nivolumab | QVQLVESGGGVVQPGRSLRLDCKASGITFSNSGMHWVRQAPGKGLEWVAVIWYD |
| | heavy chain | GSKRYYADSVKGRFTISRDNSKNTLFLQMNSLRAEDTAVYYCATNDDYWGQGTL |
| | variable region | VTVSS |
| 101 | Nivolumab | ASTKGPSVFPLAPCSRSTSESTAALGCLVKDYFPEPVTVSWNSGALTSGVHTFP |
| | heavy chain | AVLQSSGLYSLSSVVTVPSSSLGTKTYTCNVDHKPSNTKVDKRVESKYGPPCPP |
| | constant region | CPAPEFLGGPSVFLFPPKPKDTLMISRTPEVTCVVVDVSQEDPEVQFNWYVDGV |
| | | EVHNAKTKPREEQFNSTYRVVSVLTVLHQDWLNGKEYKCKVSNKGLPSSIEKTI |
| | | SKAKGQPREPQVYTLPPSQEEMTKNQVSLTCLVKGFYPSIDAVEWESNGQPENN |
| | | YKTTPPVLDSDGSFFLYSRLTVDKSRWQEGNVFSCSVMHEALHNHYTQKSLSLS |
| | | LGK |
| 102 | Nivolumab | EIVLTQSPATLSLSPGERATLSCRASQSVSSYLAWYQQKPGQAPRLLIYDASNR |
| | light chain | ATGIPARFSGSGSGTDFTLTISSLEPEDFAVYYCQQSSNWPRTFGQGTKVEIK |
| | variable region | |
| 103 | Nivolumab | RTVAAPSVFIFPPSDEQLKSGTASVVCLLNNFYPREAKVQWKVDNALQSGNSQE |
| | light chain | SVTEQDSKDSTYSLSSTLTLSKADYEKHKVYACEVTHQGLSSPVTKSFNRGEC |
| | constant region | |
| 104 | Nivolumab | QVQLVESGGGVVQPGRSLRLDCKASGITFS |
| | heavy chain | |
| | variable | |
| | region FR1 | |
| 105 | Nivolumab | NSGMH |
| | heavy chain | |
| | variable region | |
| | CDR1 | |
| 106 | Nivolumab | WVRQAPGKGLEWVA |
| | heavy chain | |
| | variable region | |
| | FR2 | |
| 107 | Nivolumab | VIWYDGSKRYYADSVKG |
| | heavy chain | |
| | variable region | |
| | CDR2 | |
| 108 | Nivolumab | RFTISRDNSKNTLFLQMNSLRAEDTAVYYCAT |
| | heavy chain | |
| | variable region | |
| | FR3 | |
| 109 | Nivolumab | NDDY |
| | heavy chain | |
| | variable region | |
| | CDR3 | |
| 110 | Nivolumab | WGQGTLVTVSS |
| | heavy chain | |
| | variable region | |
| | FR4 | |
| 111 | Nivolumab | EIVLTQSPATLSLSPGERATLSC |
| | light chain | |
| | variable region | |
| | FR1 | |
| 112 | Nivolumab | RASQSVSSYLA |
| | light chain | |
| | variable region | |
| | CDR1 | |
| 113 | Nivolumab | WYQQKPGQAPRLLIY |
| | light chain | |
| | variable region | |
| | FR2 | |
| 114 | Nivolumab | DASNRAT |
| | light chain | |
| | variable region | |
| | CDR2 | |
| 115 | Nivolumab | GIPARFSGSGSGTDFTLTISSLEPEDFAVYYC |
| | light chain | |
| | variable region | |
| | FR3 | |
| 116 | Nivolumab | QQSSNWPRT |
| | light chain | |
| | variable region | |
| | CDR3 | |
| 117 | Nivolumab | FGQGTKVEIK |
| | light chain | |
| | variable region | |
| | FR4 | |
| 118 | GITR binding | EVQLLESGGGEVQPGGSLRLSCAASGSVFSIDAMGWYRQAPGKGRELVAVLSGI |
| | polypeptide | SSAKYAASAPGRFTISRDNAKNTVYLQMSSLRAEDTAVYYCYADVSTGWGRDAH |
| | 2x hzC06v3.9 | GYWGQGTLVTVKPGGSGGSEVQLLESGGGEVQPGGSLRLSCAASGSVFSIDAMG |
| | IgG1-Fc | WYRQAPGKQRELVAVLSGISSAKYAASAPGRFTISRDNAKNTVYLQMSSLRAED |
| | | TAVYYCYADVSTGWGRDAHGYWGQGTLVTVKPGGGGDKTHTCPPCPAPELLGGP |
| | | SVFLFPPKPKDTLMISRTPEVTCVVVDVSHEDPEVKFNWYVDGVEVHNAKTKPR |
| | | EEQYNSTYRVVSVLTVLHQDWLNGKEYKCKVSNKALPAPIEKTISKAKGQPREP |
| | | QVYTLPPSRDELTKNQVSLTCLVKGFYPSDIAVEWESNGQPENNYKTTPPVLDS |
| | | DGSFFLYSKLTVDKSRWQQGNVFSCSVMHEALHNHYTQKSLSLSPGK |
| 119 | hzC06v3.9 | EVQLLESGGGEVQPGGSLRLSCAASGSVFSIDAMGWYRQAPGKQRELVAVLSGI |
| | | SSAKYAASAPGRFTISRDNAKNTVYLQMSSLRAEDTAVYYCYADVSTGWGRDAH |
| | | GYWGQGTLVTV |
| 120 | hzC06v3.9 | SGSVFSIDAM |
| | CDR1 | |
| 121 | hzC06v3.9 | LSGISSAK |
| | CDR2 | |
| 122 | hzC06v3.9 | YADVSTGWGRDAHGYW |
| | CDR3 | |
| 123 | Human | PAPE [symbol] GPS VFLFPPKPKD TLMISRPTEV TCVVVDVSHE |
| | IgGI Fc | DPEVKFNWYV DGVEVHNAKT KPREEQY [symbol] ST YRVVSVLTVL HQDWLNGKEY |
| | | KCKVSNKALP APIEKTISKA KGQPREPQVY TLPPSRDELT KNQVSLTCLV |
| | | KGFYPSDIAV EWESNGQPEN NYKTTPPVLD SDGSFFLYSK LTVDKSRWQQ |
| | | GNVFSCSVMH EALHNHYTQK SLSLSPGK |
| 124 | Human | PAPGGPSVFL FPPKPKDTLM ISRTPEVTCV VVDVSHEDPE VKFNWYVGDV |
| | IgG1 Fc | EVHNAKTKPR EEQYNSTYRV VSVLTVLHQD WLNGKEYKCK VSNKALPAPI |
| | deletion | EKTISKAKGQ PREPQVYTLP PSRDELTKNQ VSLTCLVKGF YPSDIAVEWE |
| | mutant at | SNGQPENNYK TTPPVLDSDG SFFLYSKLTV DKSRWQQGNV FSCSVMHEAL |
| | E233, L234, | HNHYTQKSLS LSPGK |
| | L235 | |
| 125 | Human | PAPPVAGPSV FLFPPKPKDT LMISRTPEVT CVVVDVSHED PEVQFNWYVD |
| | IgG2 Fc | GVEVHNAKTK PREEQF [symbol] STF RVVSVLTVVH QDWLNGKEYK CKVSNKGLPA |
| | | PIEKTISKTK GQPREPQVYT LPPSREEMTK NQVSLTCLVK GFYPSDISVE |
| | | WESNGQPENN YKTTPPMLDS DGSFFLYSKL TVDKSRWQQG NVFSCSVMHE |
| | | ALHNHYTQKS LSLSPGK |
| 126 | Human | PAPELLGGPS VFLFPPKPKD TLMISRTPEV TCVVVDVSHE DPEVQFKWYV |
| | IgG3 Fc | DGVEVHNAKT KPREEQY [symbol] ST FRVVSVLTVL HQDWLNGKEY KCKVSNKALP |
| | | APIEKTISKT KGQPREPQVY TLPPSREEMT KNQVSLTCLV KGFYPSDIAV |
| | | EWESSGQPEN NYNTTPPMLD SDGSFFLYSK LTVDKSRWQQ GNIFSCSVMH |
| | | EALHNF [symbol] TQK SLSLSPGK |
| 128 | Human | PAPEF [symbol] GGPS VFLFPPKPKD TLMISRTPEV TCVVVDVSQE DPEVQFNWYV |
| | IgG4 Fc | DGVEVHNAKT KPREEQF [symbol] ST YRVVSVLTVL HQDWLNGKEY KCKVSNKGLP |
| | | SSIEKTISKA GQPREPQVY TLPPSQEEMT KNQVSLTCLV KGFYPSIDAV |
| | | EWESNGQPEN NYKTTPPVLD SDGSFFLYSR LTVDKSRWQE GNVFSCSVMH |
| | | EALHNHYTQK SLSLSLGK |
| 128 | Human | PAPELLGGPS VFLFPPKPKD TLMISRTPEV TCVVVDVSQE DPEVQFNWYV |
| | IgG4 Fc | DGVEVHNAKT KPREEQF [symbol] ST YRVVSVLTVL HQDWLNGKEY KCKVSNKGLP |
| | | SSIEKTISKA KGQPREPQVY TLPPSQEEMT KNQVSLTCLV KGFYPSDIAV |
| | | EWESNGQPEN NYKTTPPVLD SDGSFFLYSR LTVDKSRWQE GNVFSCSVMH |
| | | EALHNHYTQK SLSLSLGK |
| 129 | IgG hinge | EPKSSDKTHTCPPC |
| | region | |
| 130 | IgG hinge | DKTHTCPPC |
| | region | |
| 131 | IgG hinge | ESKYGPPCPPC |
| | region | |
| 132 | Carboxy- | GQGTLVTVKPGG |
| | terminal | |
| | sequence | |
| 133 | Carboxy- | GQGTLVTVEPGG |
| | terminal | |
| | sequence | |
| 134 | Linker | GGSGGS |
| | sequence | |
| 135 | Linker | GGSGGSGGS |
| | sequence | |
| 136 | Linker | GGSGGSGGSGGS |
| | sequence | |
| 137 | Linker | GGSGGSGGSGGSGGS |
| | sequence | |
| 138 | Linker | GGGG |
| | sequence | |
| 139 | Linker | GGGGG |
| | sequence | |
| 140 | Linker | GGGGGG |
| | sequence | |

1. A method of treating cancer in a subject comprising administering to the subject an anti-Colony Stimulating Factor 1 Receptor (CSF1R) antibody and an anti-Glucocorticoid-Induced TNFR-Related protein (GITR) antibody,wherein the anti-GITR antibody is selected from:
a) an antibody comprising a GITR binding domain (GITR-BD) comprising a CDR1 comprising the sequence of SEQ ID NO: 120, a CDR2 comprising the sequence of SEQ ID NO: 121, and a CDR3 comprising the sequence of SEQ ID NO: 122;
b) an antibody comprising a GITR-BD comprising the sequence of SEQ ID NO: 119;
c) a tetravalent molecule comprising two copies of a polypeptide having the structure (GITR-BD)-Linker-(GITR-BD)-Linker-Hinge-Fc, wherein (i) the GITR-BD comprises a CDR1 comprising the sequence of SEQ ID NO: 120, a CDR2 comprising the sequence of SEQ ID NO: 121, and a CDR3 comprising the sequence of SEQ ID NO: 122, (ii) the Linker is a polypeptide, (iii) the Hinge is a polypeptide derived from an immunoglobulin hinge region, and (iv) the Fc is an immunoglobulin Fc polypeptide;
d) a tetravalent molecule comprising two copies of a polypeptide having the structure (GITR-BD)-Linker-(GITR-BD)-Linker-Hinge-Fc, wherein (i) the GITR-BD comprises the amino acid sequence of SEQ ID NO:119, (ii) the Linker is a polypeptide, (iii) the Hinge is a polypeptide derived from an immunoglobulin hinge region, and (iv) the Fc is an immunoglobulin Fc polypeptide; and
e) a tetravalent molecule comprising two copies of a polypeptide comprising the sequence of SEQ ID NO: 118.

2. The method of claim 1, wherein the anti-CSF1R antibody is selected from:
a) an antibody comprising a heavy chain comprising the sequence of SEQ ID NO: 39 and a light chain comprising the sequence of SEQ ID NO: 46;
b) an antibody comprising a heavy chain comprising a heavy chain (HC) complementarity determining region 1 (CDR1) comprising the sequence of SEQ ID NO: 15, an HC CDR2 comprising the sequence of SEQ ID NO: 16, and an HC CDR3 comprising the sequence of SEQ ID NO: 17, and a light chain comprising a light chain (LC) CDR1 comprising the sequence of SEQ ID NO: 18, a LC CDR2 comprising the sequence of SEQ ID NO: 19, and a LC CDR3 comprising the sequence of SEQ ID NO: 20; and
c) an antibody comprising a heavy chain comprising the sequence of SEQ ID NO: 53 and a light chain comprising the sequence of SEQ ID NO: 60.

3. The method of claim 1 or 2, wherein the anti-GITR antibody is a tetravalent molecule having the structure (GITR-BD)-Linker-(GITR-BD)-Linker-Hinge-Fc, wherein (i) the GITR-BD comprises (a) a CDR1 comprising the sequence of SEQ ID NO: 120, a CDR2 comprising the sequence of SEQ ID NO: 121, and a CDR3 comprising the sequence of SEQ ID NO: 122 or (b) the sequence of SEQ ID NO:119, (ii) the Linker is a polypeptide comprising a sequence selected from SEQ ID NOs: 134-140, (iii) the Hinge is a polypeptide comprising a sequence selected from SEQ ID NOs: 129-133, and (iv) the Fc is an immunoglobulin Fc polypeptide comprising a sequence selected from SEQ ID NOs: 123-128.

4. The method of any claims 1-3, wherein the anti-CSF1R antibody is a humanized antibody or is selected from a Fab, an Fv, an scFv, a Fab′, and a (Fab′)₂.

5. The method of any one of claims 1-4, wherein the anti-CSF1R antibody and the anti-GITR antibody are administered concurrently or sequentially.

6. The method of any one of claims 1-5, wherein the anti-CSF1R antibody and the anti-GITR antibody are administered once every week, once every 2 weeks, once every 3 weeks, once every 4 weeks, or once every 5 weeks.

7. The method any one of claims 1-6, wherein the anti-CSF1R antibody is administered at a dose of 0.1, 0.3, 0.5, 1, 2, 3, 4, 5, or 10 mg/kg.

8. The method of claim 7, wherein the anti-CSF1R antibody is administered at a dose of 1, 2, 3, or 4 mg/kg every 2 weeks or every 3 weeks.

9. The method of any one of the preceding claims, wherein the cancer is selected from non-small cell lung cancer, melanoma, squamous cell carcinoma of the head and neck, ovarian cancer, pancreatic cancer, renal cell carcinoma, hepatocellular carcinoma, bladder cancer, malignant glioma, colorectal cancer, and endometrial cancer.

10. The method of any one of the preceding claims, wherein the cancer is recurrent or progressive after a therapy selected from one or more of surgery, chemotherapy, and radiation therapy.

11. The method of any one of the preceding claims, wherein the anti-CSF1R antibody blocks binding of both CSF1 and IL-34 to CSF1R.

12. The method of any one of the preceding claims, wherein the anti-CSF1R antibody inhibits ligand-induced CSF1R phosphorylation in vitro.

13. The method of any one of the preceding claims, wherein administration of the anti-CSF1R antibody and the anti-GITR antibody results in a synergistic effect.

14. The method of claim 13, wherein administration of the anti-CSF1R antibody and the anti-GITR antibody results in a synergistic inhibition of tumor growth in a mouse xenograft or syngeneic cancer model.

15. The method of any one of claims 1-14, wherein the method further comprises administering at least one chemotherapeutic agent.

16. A method of treating cancer in a subject comprising administering to the subject an anti-Programmed cell Death 1 (PD-1) antibody and an anti-Glucocorticoid-Induced TNFR-Related protein (GITR) antibody, wherein the anti-GITR antibody is selected from:
a) an antibody comprising a GITR binding domain (GITR-BD) comprising a CDR1 comprising the sequence of SEQ ID NO: 120, a CDR2 comprising the sequence of SEQ ID NO: 121, and a CDR3 comprising the sequence of SEQ ID NO: 122;
b) an antibody comprising a GITR-BD comprising the sequence of SEQ ID NO: 119;
c) a tetravalent molecule comprising two copies of a polypeptide having the structure (GITR-BD)-Linker-(GITR-BD)-Linker-Hinge-Fc, wherein (i) the GITR-BD comprises a CDR1 comprising the sequence of SEQ ID NO: 120, a CDR2 comprising the sequence of SEQ ID NO: 121, and a CDR3 comprising the sequence of SEQ ID NO: 122, (ii) the Linker is a polypeptide, (iii) the Hinge is a polypeptide derived from an immunoglobulin hinge region, and (iv) the Fc is an immunoglobulin Fc polypeptide;
d) a tetravalent molecule comprising two copies of a polypeptide having the structure (GITR-BD)-Linker-(GITR-BD)-Linker-Hinge-Fc, wherein (i) the GITR-BD comprises the amino acid sequence of SEQ ID NO:119, (ii) the Linker is a polypeptide, (iii) the Hinge is a polypeptide derived from an immunoglobulin hinge region, and (iv) the Fc is an immunoglobulin Fc polypeptide; and
e) a tetravalent molecule comprising two copies of a polypeptide comprising the sequence of SEQ ID NO: 118.

17. The method of claim 16, wherein the anti-PD-1 antibody is selected from:
a) an antibody comprising a heavy chain comprising the sequence of SEQ ID NO: 100 and a light chain comprising the sequence of SEQ ID NO: 102;
b) an antibody comprising a heavy chain comprising a heavy chain (HC) complementarity determining region 1 (CDR1) having the sequence of SEQ ID NO: 105, an HC CDR2 having the sequence of SEQ ID NO: 107, and an HC CDR3 having the sequence of SEQ ID NO: 109, and a light chain comprising a light chain (LC) CDR1 having the sequence of SEQ ID NO: 112, a LC CDR2 having the sequence of SEQ ID NO: 114, and a LC CDR3 having the sequence of SEQ ID NO: 116; and
c) an antibody comprising a heavy chain comprising the sequences of SEQ ID NOs: 100 and 101 and a light chain comprising the sequences of SEQ ID NOs: 102 and 103

18. The method of claim 16 or 17, wherein the anti-GITR antibody is a tetravalent molecule having the structure (GITR-BD)-Linker-(GITR-BD)-Linker-Hinge-Fc, wherein (i) the GITR-BD comprises (a) a CDR1 comprising the sequence of SEQ ID NO: 120, a CDR2 comprising the sequence of SEQ ID NO: 121, and a CDR3 comprising the sequence of SEQ ID NO: 122 or (b) the sequence of SEQ ID NO: 119, (ii) the Linker is a polypeptide comprising a sequence selected from SEQ ID NOs: 134-140, (iii) the Hinge is a polypeptide comprising a sequence selected from SEQ ID NOs: 129-133, and (iv) the Fc is an immunoglobulin Fc polypeptide comprising a sequence selected from SEQ ID NOs: 123-128.

19. The method of any of claims 16-18, wherein the anti-PD-1 antibody is a humanized antibody or is selected from a Fab, an Fv, an scFv, a Fab′, and a (Fab′)₂.

20. The method of claim 19, wherein the anti-PD-1 antibody is nivolumab.

21. The method of any one of claims 16-20, wherein the anti-PD-1 antibody and the anti-GITR antibody are administered concurrently or sequentially.

22. The method of any one of claims 16-21, wherein the anti-PD-1 antibody and the anti-GITR antibody are administered once every week, once every 2 weeks, once every 3 weeks, once every 4 weeks, or once every 5 weeks.

23. The method of any one of claims 16-22, wherein the anti-PD-1 antibody is administered at a dose of 0.5, 1, 2, 3, 4, 5, or 10 mg/kg.

24. The method of claim 23, wherein the anti-PD-1 antibody is nivolumab and wherein the nivolumab is administered at a dose of 3 mg/kg every 2 weeks or at a flat dose of 240 mg every 2 weeks.

25. The method of any one of claims 16-24, wherein the cancer is selected from non-small cell lung cancer, melanoma, squamous cell carcinoma of the head and neck, ovarian cancer, pancreatic cancer, renal cell carcinoma, hepatocellular carcinoma, bladder cancer, malignant glioma, colorectal cancer, and endometrial cancer.

26. The method of any one of claims 16-25, wherein the cancer is recurrent or progressive after a therapy selected from one or more of surgery, chemotherapy, and radiation therapy.

27. The method of any one of claims 16-26, wherein administration of the anti-PD-1 antibody and the anti-GITR antibody results in a synergistic effect.

28. The method of claim 27, wherein administration of the anti-PD-1 antibody and the anti-GITR antibody results in a synergistic inhibition of tumor growth in a mouse xenograft or syngeneic cancer model.

29. The method of any one of claims 16-28, wherein the method further comprises administering at least one chemotherapeutic agent.

30. The method of any one of the preceding claims, wherein the subject has previously received PD-1/PD-L1 inhibitor therapy.

31. The method of claim 30, wherein the subject is a PD-1/PD-L1 inhibitor inadequate responder or is refractory to a PD-1/PD-L1 inhibitor after at least 2 doses.

32. A composition comprising an anti-GITR antibody for use in a method of treating cancer according to any one of claims 1-31; wherein the anti-GITR antibody is selected from:
a) an antibody comprising a GITR binding domain (GITR-BD) comprising a CDR1 comprising the sequence of SEQ ID NO: 120, a CDR2 comprising the sequence of SEQ ID NO: 121, and a CDR3 comprising the sequence of SEQ ID NO: 122;
b) an antibody comprising a GITR-BD comprising the sequence of SEQ ID NO: 119;
c) a tetravalent molecule comprising two copies of a polypeptide having the structure (GITR-BD)-Linker-(GITR-BD)-Linker-Hinge-Fc, wherein (i) the GITR-BD comprises a CDR1 comprising the sequence of SEQ ID NO: 120, a CDR2 comprising the sequence of SEQ ID NO: 121, and a CDR3 comprising the sequence of SEQ ID NO: 122, (ii) the Linker is a polypeptide, (iii) the Hinge is a polypeptide derived from an immunoglobulin hinge region, and (iv) the Fc is an immunoglobulin Fc polypeptide;
d) a tetravalent molecule comprising two copies of a polypeptide having the structure (GITR-BD)-Linker-(GITR-BD)-Linker-Hinge-Fc, wherein (i) the GITR-BD comprises the amino acid sequence of SEQ ID NO:119, (ii) the Linker is a polypeptide, (iii) the Hinge is a polypeptide derived from an immunoglobulin hinge region, and (iv) the Fc is an immunoglobulin Fc polypeptide; and
e) a tetravalent molecule comprising two copies of a polypeptide comprising the sequence of SEQ ID NO: 118.

33. Use of an anti-GITR antibody for preparation of a medicament for treating cancer in a subject according to the steps and/or conditions of any one of claims 1-31; wherein the anti-GITR antibody is selected from:
a) an antibody comprising a GITR binding domain (GITR-BD) comprising a CDR1 comprising the sequence of SEQ ID NO: 120, a CDR2 comprising the sequence of SEQ ID NO: 121, and a CDR3 comprising the sequence of SEQ ID NO: 122;
b) an antibody comprising a GITR-BD comprising the sequence of SEQ ID NO: 119;
c) a tetravalent molecule comprising two copies of a polypeptide having the structure (GITR-BD)-Linker-(GITR-BD)-Linker-Hinge-Fc, wherein (i) the GITR-BD comprises a CDR1 comprising the sequence of SEQ ID NO: 120, a CDR2 comprising the sequence of SEQ ID NO: 121, and a CDR3 comprising the sequence of SEQ ID NO: 122, (ii) the Linker is a polypeptide, (iii) the Hinge is a polypeptide derived from an immunoglobulin hinge region, and (iv) the Fc is an immunoglobulin Fc polypeptide;
d) a tetravalent molecule comprising two copies of a polypeptide having the structure (GITR-BD)-Linker-(GITR-BD)-Linker-Hinge-Fc, wherein (i) the GITR-BD comprises the amino acid sequence of SEQ ID NO:119, (ii) the Linker is a polypeptide, (iii) the Hinge is a polypeptide derived from an immunoglobulin hinge region, and (iv) the Fc is an immunoglobulin Fc polypeptide; and
e) a tetravalent molecule comprising two copies of a polypeptide comprising the sequence of SEQ ID NO: 118.

34. A composition comprising an anti-GITR antibody and an anti-CSF1R antibody for use in a method of treating cancer according to any one of claim 1-15, 30, or 31;
wherein the anti-CSF1R antibody is selected from:
a) an antibody comprising a heavy chain comprising the sequence of SEQ ID NO: 39 and a light chain comprising the sequence of SEQ ID NO: 46;
b) an antibody comprising a heavy chain comprising a heavy chain (HC) complementarity determining region 1 (CDR1) comprising the sequence of SEQ ID NO: 15, an HC CDR2 comprising the sequence of SEQ ID NO: 16, and an HC CDR3 comprising the sequence of SEQ ID NO: 17, and a light chain comprising a light chain (LC) CDR1 comprising the sequence of SEQ ID NO: 18, a LC CDR2 comprising the sequence of SEQ ID NO: 19, and a LC CDR3 comprising the sequence of SEQ ID NO: 20; and
c) an antibody comprising a heavy chain comprising the sequence of SEQ ID NO: 53 and a light chain comprising the sequence of SEQ ID NO: 60;
and wherein the anti-GITR antibody is selected from:
a) an antibody comprising a GITR binding domain (GITR-BD) comprising a CDR1 comprising the sequence of SEQ ID NO: 120, a CDR2 comprising the sequence of SEQ ID NO: 121, and a CDR3 comprising the sequence of SEQ ID NO: 122;
b) an antibody comprising a GITR-BD comprising the sequence of SEQ ID NO: 119;
c) a tetravalent molecule comprising two copies of a polypeptide having the structure (GITR-BD)-Linker-(GITR-BD)-Linker-Hinge-Fc, wherein (i) the GITR-BD comprises a CDR1 comprising the sequence of SEQ ID NO: 120, a CDR2 comprising the sequence of SEQ ID NO: 121, and a CDR3 comprising the sequence of SEQ ID NO: 122, (ii) the Linker is a polypeptide, (iii) the Hinge is a polypeptide derived from an immunoglobulin hinge region, and (iv) the Fc is an immunoglobulin Fc polypeptide;
d) a tetravalent molecule comprising two copies of a polypeptide having the structure (GITR-BD)-Linker-(GITR-BD)-Linker-Hinge-Fc, wherein (i) the GITR-BD comprises the amino acid sequence of SEQ ID NO:119, (ii) the Linker is a polypeptide, (iii) the Hinge is a polypeptide derived from an immunoglobulin hinge region, and (iv) the Fc is an immunoglobulin Fc polypeptide; and
e) a tetravalent molecule comprising two copies of a polypeptide comprising the sequence of SEQ ID NO: 118.

35. Use of a composition comprising an anti-GITR antibody and an anti-CSF1R antibody for preparation of a medicament for treating cancer in a subject according to the steps and/or conditions of any one of claim 1-15, 30, or 31;
wherein the anti-CSF1R antibody is selected from:
a) an antibody comprising a heavy chain comprising the sequence of SEQ ID NO: 39 and a light chain comprising the sequence of SEQ ID NO: 46;
b) an antibody comprising a heavy chain comprising a heavy chain (HC) complementarity determining region 1 (CDR1) comprising the sequence of SEQ ID NO: 15, an HC CDR2 comprising the sequence of SEQ ID NO: 16, and an HC CDR3 comprising the sequence of SEQ ID NO: 17, and a light chain comprising a light chain (LC) CDR1 comprising the sequence of SEQ ID NO: 18, a LC CDR2 comprising the sequence of SEQ ID NO: 19, and a LC CDR3 comprising the sequence of SEQ ID NO: 20; and
c) an antibody comprising a heavy chain comprising the sequence of SEQ ID NO: 53 and a light chain comprising the sequence of SEQ ID NO: 60;
and wherein the anti-GITR antibody is selected from:
a) an antibody comprising a GITR binding domain (GITR-BD) comprising a CDR1 comprising the sequence of SEQ ID NO: 120, a CDR2 comprising the sequence of SEQ ID NO: 121, and a CDR3 comprising the sequence of SEQ ID NO: 122;
b) an antibody comprising a GITR-BD comprising the sequence of SEQ ID NO: 119;
c) a tetravalent molecule comprising two copies of a polypeptide having the structure (GITR-BD)-Linker-(GITR-BD)-Linker-Hinge-Fc, wherein (i) the GITR-BD comprises a CDR1 comprising the sequence of SEQ ID NO: 120, a CDR2 comprising the sequence of SEQ ID NO: 121, and a CDR3 comprising the sequence of SEQ ID NO: 122, (ii) the Linker is a polypeptide, (iii) the Hinge is a polypeptide derived from an immunoglobulin hinge region, and (iv) the Fc is an immunoglobulin Fc polypeptide;
d) a tetravalent molecule comprising two copies of a polypeptide having the structure (GITR-BD)-Linker-(GITR-BD)-Linker-Hinge-Fc, wherein (i) the GITR-BD comprises the amino acid sequence of SEQ ID NO:119, (ii) the Linker is a polypeptide, (iii) the Hinge is a polypeptide derived from an immunoglobulin hinge region, and (iv) the Fc is an immunoglobulin Fc polypeptide; and
e) a tetravalent molecule comprising two copies of a polypeptide comprising the sequence of SEQ ID NO: 118.

36. A composition comprising an anti-GITR antibody and an anti-PD-1 antibody for use in a method of treating cancer according to any one of claims 16-31;
wherein the anti-PD-1 antibody is selected from:
a) an antibody comprising a heavy chain comprising the sequence of SEQ ID NO: 100 and a light chain comprising the sequence of SEQ ID NO: 102;
b) an antibody comprising a heavy chain comprising a heavy chain (HC) complementarity determining region 1 (CDR1) having the sequence of SEQ ID NO: 105, an HC CDR2 having the sequence of SEQ ID NO: 107, and an HC CDR3 having the sequence of SEQ ID NO: 109, and a light chain comprising a light chain (LC) CDR1 having the sequence of SEQ ID NO: 112, a LC CDR2 having the sequence of SEQ ID NO: 114, and a LC CDR3 having the sequence of SEQ ID NO: 116; and
c) an antibody comprising a heavy chain comprising the sequences of SEQ ID NOs: 100 and 101 and a light chain comprising the sequences of SEQ ID NOs: 102 and 103;
and wherein the anti-GITR antibody is selected from:
a) an antibody comprising a GITR binding domain (GITR-BD) comprising a CDR1 comprising the sequence of SEQ ID NO: 120, a CDR2 comprising the sequence of SEQ ID NO: 121, and a CDR3 comprising the sequence of SEQ ID NO: 122;
b) an antibody comprising a GITR-BD comprising the sequence of SEQ ID NO: 119;
c) a tetravalent molecule comprising two copies of a polypeptide having the structure (GITR-BD)-Linker-(GITR-BD)-Linker-Hinge-Fc, wherein (i) the GITR-BD comprises a CDR1 comprising the sequence of SEQ ID NO: 120, a CDR2 comprising the sequence of SEQ ID NO: 121, and a CDR3 comprising the sequence of SEQ ID NO: 122, (ii) the Linker is a polypeptide, (iii) the Hinge is a polypeptide derived from an immunoglobulin hinge region, and (iv) the Fc is an immunoglobulin Fc polypeptide;
d) a tetravalent molecule comprising two copies of a polypeptide having the structure (GITR-BD)-Linker-(GITR-BD)-Linker-Hinge-Fc, wherein (i) the GITR-BD comprises the amino acid sequence of SEQ ID NO:119, (ii) the Linker is a polypeptide, (iii) the Hinge is a polypeptide derived from an immunoglobulin hinge region, and (iv) the Fc is an immunoglobulin Fc polypeptide; and
e) a tetravalent molecule comprising two copies of a polypeptide comprising the sequence of SEQ ID NO: 118.

37. Use of the composition comprising an anti-GITR antibody and an anti-PD-1 antibody for preparation of a medicament for treating cancer in a subject according to the steps and/or conditions of any one of claims 16-31;
wherein the anti-PD-1 antibody is selected from:
a) an antibody comprising a heavy chain comprising the sequence of SEQ ID NO: 100 and a light chain comprising the sequence of SEQ ID NO: 102;
b) an antibody comprising a heavy chain comprising a heavy chain (HC) complementarity determining region 1 (CDR1) having the sequence of SEQ ID NO: 105, an HC CDR2 having the sequence of SEQ ID NO: 107, and an HC CDR3 having the sequence of SEQ ID NO: 109, and a light chain comprising a light chain (LC) CDR1 having the sequence of SEQ ID NO: 112, a LC CDR2 having the sequence of SEQ ID NO: 114, and a LC CDR3 having the sequence of SEQ ID NO: 116; and
c) an antibody comprising a heavy chain comprising the sequences of SEQ ID NOs: 100 and 101 and a light chain comprising the sequences of SEQ ID NOs: 102 and 103;
and wherein the anti-GITR antibody is selected from:
a) an antibody comprising a GITR binding domain (GITR-BD) comprising a CDR1 comprising the sequence of SEQ ID NO: 120, a CDR2 comprising the sequence of SEQ ID NO: 121, and a CDR3 comprising the sequence of SEQ ID NO: 122;
b) an antibody comprising a GITR-BD comprising the sequence of SEQ ID NO: 119;
c) a tetravalent molecule comprising two copies of a polypeptide having the structure (GITR-BD)-Linker-(GITR-BD)-Linker-Hinge-Fc, wherein (i) the GITR-BD comprises a CDR1 comprising the sequence of SEQ ID NO: 120, a CDR2 comprising the sequence of SEQ ID NO: 121, and a CDR3 comprising the sequence of SEQ ID NO: 122, (ii) the Linker is a polypeptide, (iii) the Hinge is a polypeptide derived from an immunoglobulin hinge region, and (iv) the Fc is an immunoglobulin Fc polypeptide;
d) a tetravalent molecule comprising two copies of a polypeptide having the structure (GITR-BD)-Linker-(GITR-BD)-Linker-Hinge-Fc, wherein (i) the GITR-BD comprises the amino acid sequence of SEQ ID NO:119, (ii) the Linker is a polypeptide, (iii) the Hinge is a polypeptide derived from an immunoglobulin hinge region, and (iv) the Fc is an immunoglobulin Fc polypeptide; and
e) a tetravalent molecule comprising two copies of a polypeptide comprising the sequence of SEQ ID NO: 118.

38. The composition of any one of claim 32, 34, or 36, wherein the composition further comprises at least one chemotherapeutic agent.

39. The use of any one of claim 33, 35, or 37, wherein the treatment further comprises administering at least one chemotherapeutic agent.

40. A method of treating pancreatic cancer in a subject comprising administering to the subject an anti-Colony Stimulating Factor 1 Receptor (CSF1R) antibody and an anti-Glucocorticoid-Induced TNFR-Related protein (GITR) antibody, wherein the anti-CSF1R antibody is selected from:
a) an antibody comprising a heavy chain comprising the sequence of SEQ ID NO: 39 and a light chain comprising the sequence of SEQ ID NO: 46;
b) an antibody comprising a heavy chain comprising a heavy chain (HC) complementarity determining region 1 (CDR1) comprising the sequence of SEQ ID NO: 15, an HC CDR2 comprising the sequence of SEQ ID NO: 16, and an HC CDR3 comprising the sequence of SEQ ID NO: 17, and a light chain comprising a light chain (LC) CDR1 comprising the sequence of SEQ ID NO: 18, a LC CDR2 comprising the sequence of SEQ ID NO: 19, and a LC CDR3 comprising the sequence of SEQ ID NO: 20; and
c) an antibody comprising a heavy chain comprising the sequence of SEQ ID NO: 53 and a light chain comprising the sequence of SEQ ID NO: 60.

41. A method of treating pancreatic cancer in a subject comprising administering to the subject an anti-Colony Stimulating Factor 1 Receptor (CSF1R) antibody and an anti-Glucocorticoid-Induced TNFR-Related protein (GITR) antibody, wherein the anti-GITR antibody is a tetravalent molecule having the structure (GITR-BD)-Linker-(GITR-BD)-Linker-Hinge-Fc, wherein (i) the GITR-BD comprises (a) a CDR1 comprising the sequence of SEQ ID NO: 120, a CDR2 comprising the sequence of SEQ ID NO: 121, and a CDR3 comprising the sequence of SEQ ID NO: 122 or (b) the sequence of SEQ ID NO:119, (ii) the Linker is a polypeptide comprising a sequence selected from SEQ ID NOs: 134-140, (iii) the Hinge is a polypeptide comprising a sequence selected from SEQ ID NOs: 129-133, and (iv) the Fc is an immunoglobulin Fc polypeptide comprising a sequence selected from SEQ ID NOs: 123-128.

42. The method of claim 41, wherein the anti-CSF1R antibody is selected from:
a) an antibody comprising a heavy chain comprising the sequence of SEQ ID NO: 39 and a light chain comprising the sequence of SEQ ID NO: 46;
b) an antibody comprising a heavy chain comprising a heavy chain (HC) complementarity determining region 1 (CDR1) comprising the sequence of SEQ ID NO: 15, an HC CDR2 comprising the sequence of SEQ ID NO: 16, and an HC CDR3 comprising the sequence of SEQ ID NO: 17, and a light chain comprising a light chain (LC) CDR1 comprising the sequence of SEQ ID NO: 18, a LC CDR2 comprising the sequence of SEQ ID NO: 19, and a LC CDR3 comprising the sequence of SEQ ID NO: 20; and
c) an antibody comprising a heavy chain comprising the sequence of SEQ ID NO: 53 and a light chain comprising the sequence of SEQ ID NO: 60.

43. The method of any of claims 40-42, wherein the anti-CSF1R antibody is a humanized antibody or is selected from a Fab, an Fv, an scFv, a Fab′, and a (Fab′)₂.

44. The method of any one of claims 40-43, wherein the anti-CSF1R antibody and the anti-GITR antibody are administered concurrently or sequentially.

45. The method of any one of claims 40-44, wherein the anti-CSF1R antibody and the anti-GITR antibody are administered once every week, once every 2 weeks, once every 3 weeks, once every 4 weeks, or once every 5 weeks.

46. The method any one of claims 40-45, wherein the anti-CSF1R antibody is administered at a dose of 0.1, 0.3, 0.5, 1, 2, 3, 4, 5, or 10 mg/kg.

47. The method of claim 46, wherein the anti-CSF1R antibody is administered at a dose of 1, 2, 3, or 4 mg/kg every 2 weeks or every 3 weeks.

48. The method of any one of claims 40-47, wherein the anti-CSF1R antibody blocks binding of both CSF1 and IL-34 to CSF1R.

49. The method of any one of claims 40-48, wherein the anti-CSF1R antibody inhibits ligand-induced CSF1R phosphorylation in vitro.

50. The method of any one of claims 40-49, wherein administration of the anti-CSF1R antibody and the anti-GITR antibody results in a synergistic effect.

51. The method of claim 50, wherein administration of the anti-CSF1R antibody and the anti-GITR antibody results in a synergistic inhibition of tumor growth in a mouse xenograft or syngeneic pancreatic cancer model.

52. The method of any one of claims 40-51, wherein the method further comprises administering at least one chemotherapeutic agent.

53. The method of claim 52, wherein the at least one chemotherapeutic agent is selected from gemcitabine, nab-pactlitaxel, leukovorin (folinic acid), 5-fluorouracil, irinotecan, and oxaliplatin.

54. The method of claim 53, wherein the at least one chemotherapeutic agent is selected from (a) gemcitabine (b) gemcitabine and nab-paclitaxel, and (c) FOLFIRINOX.

55. The method of claim 54, wherein the at least one chemotherapeutic agent is gemcitabine.

56. The method of any one of claims 40-55, wherein the method further comprises administering an anti-PD-1 antibody.

57. The method of claim 56, wherein the anti-PD-1 antibody is selected from:
a) an antibody comprising a heavy chain comprising the sequence of SEQ ID NO: 100 and a light chain comprising the sequence of SEQ ID NO: 102;
b) an antibody comprising a heavy chain comprising a heavy chain (HC) complementarity determining region 1 (CDR1) having the sequence of SEQ ID NO: 105, an HC CDR2 having the sequence of SEQ ID NO: 107, and an HC CDR3 having the sequence of SEQ ID NO: 109, and a light chain comprising a light chain (LC) CDR1 having the sequence of SEQ ID NO: 112, a LC CDR2 having the sequence of SEQ ID NO: 114, and a LC CDR3 having the sequence of SEQ ID NO: 116; and
c) an antibody comprising a heavy chain comprising the sequences of SEQ ID NOs: 100 and 101 and a light chain comprising the sequences of SEQ ID NOs: 102 and 103.

58. A method of treating pancreatic cancer in a subject comprising administering to the subject an anti-Colony Stimulating Factor 1 Receptor (CSF1R) antibody, an anti-Glucocorticoid-Induced TNFR-Related protein (GITR) antibody, and at least one chemotherapeutic agent selected from gemcitabine, nab-pactlitaxel, leukovorin (folinic acid), 5-fluorouracil, irinotecan, and oxaliplatin.

59. The method of claim 58, wherein the at least one chemotherapeutic agent is selected from (a) gemcitabine, (b) gemcitabine and nab-paclitaxel, and (c) FOLFIRINOX.

60. The method of claim 59, wherein the at least one chemotherapeutic agent is gemcitabine.

61. The method of any one of claims 58-60, wherein the anti-GITR antibody is selected from:
a) an antibody comprising a GITR binding domain (GITR-BD) comprising a CDR1 comprising the sequence of SEQ ID NO: 120, a CDR2 comprising the sequence of SEQ ID NO: 121, and a CDR3 comprising the sequence of SEQ ID NO: 122;
b) an antibody comprising a GITR-BD comprising the sequence of SEQ ID NO: 119;
c) a tetravalent molecule comprising two copies of a polypeptide having the structure (GITR-BD)-Linker-(GITR-BD)-Linker-Hinge-Fc, wherein (i) the GITR-BD comprises a CDR1 comprising the sequence of SEQ ID NO: 120, a CDR2 comprising the sequence of SEQ ID NO: 121, and a CDR3 comprising the sequence of SEQ ID NO: 122, (ii) the Linker is a polypeptide, (iii) the Hinge is a polypeptide derived from an immunoglobulin hinge region, and (iv) the Fc is an immunoglobulin Fc polypeptide;
d) a tetravalent molecule comprising two copies of a polypeptide having the structure (GITR-BD)-Linker-(GITR-BD)-Linker-Hinge-Fc, wherein (i) the GITR-BD comprises the amino acid sequence of SEQ ID NO:119, (ii) the Linker is a polypeptide, (iii) the Hinge is a polypeptide derived from an immunoglobulin hinge region, and (iv) the Fc is an immunoglobulin Fc polypeptide; and
e) a tetravalent molecule comprising two copies of a polypeptide comprising the sequence of SEQ ID NO: 118.

62. The method of claim 61, wherein the anti-GITR antibody is a tetravalent molecule having the structure (GITR-BD)-Linker-(GITR-BD)-Linker-Hinge-Fc, wherein (i) the GITR-BD comprises (a) a CDR1 comprising the sequence of SEQ ID NO: 120, a CDR2 comprising the sequence of SEQ ID NO: 121, and a CDR3 comprising the sequence of SEQ ID NO: 122 or (b) the sequence of SEQ ID NO:119, (ii) the Linker is a polypeptide comprising a sequence selected from SEQ ID NOs: 134-140, (iii) the Hinge is a polypeptide comprising a sequence selected from SEQ ID NOs: 129-133, and (iv) the Fc is an immunoglobulin Fc polypeptide comprising a sequence selected from SEQ ID NOs: 123-128.

63. The method of any one of claims 58-62, wherein the anti-CSF1R antibody is selected from:
a) an antibody comprising a heavy chain comprising the sequence of SEQ ID NO: 39 and a light chain comprising the sequence of SEQ ID NO: 46;
b) an antibody comprising a heavy chain comprising a heavy chain (HC) complementarity determining region 1 (CDR1) comprising the sequence of SEQ ID NO: 15, an HC CDR2 comprising the sequence of SEQ ID NO: 16, and an HC CDR3 comprising the sequence of SEQ ID NO: 17, and a light chain comprising a light chain (LC) CDR1 comprising the sequence of SEQ ID NO: 18, a LC CDR2 comprising the sequence of SEQ ID NO: 19, and a LC CDR3 comprising the sequence of SEQ ID NO: 20; and
c) an antibody comprising a heavy chain comprising the sequence of SEQ ID NO: 53 and a light chain comprising the sequence of SEQ ID NO: 60.

64. The method of any one of claims 58-63, wherein the method further comprises administering an anti-PD-1 antibody.

65. The method of claim 64, wherein the anti-PD-1 antibody is selected from:
a) an antibody comprising a heavy chain comprising the sequence of SEQ ID NO: 100 and a light chain comprising the sequence of SEQ ID NO: 102;
b) an antibody comprising a heavy chain comprising a heavy chain (HC) complementarity determining region 1 (CDR1) having the sequence of SEQ ID NO: 105, an HC CDR2 having the sequence of SEQ ID NO: 107, and an HC CDR3 having the sequence of SEQ ID NO: 109, and a light chain comprising a light chain (LC) CDR1 having the sequence of SEQ ID NO: 112, a LC CDR2 having the sequence of SEQ ID NO: 114, and a LC CDR3 having the sequence of SEQ ID NO: 116; and
c) an antibody comprising a heavy chain comprising the sequences of SEQ ID NOs: 100 and 101 and a light chain comprising the sequences of SEQ ID NOs: 102 and 103.

66. A composition comprising an anti-GITR antibody for use in a method of treating pancreatic cancer according to any one of claims 40-65.

67. The composition of claim 66, wherein the anti-GITR antibody is selected from:
a) an antibody comprising a GITR binding domain (GITR-BD) comprising a CDR1 comprising the sequence of SEQ ID NO: 120, a CDR2 comprising the sequence of SEQ ID NO: 121, and a CDR3 comprising the sequence of SEQ ID NO: 122;
b) an antibody comprising a GITR-BD comprising the sequence of SEQ ID NO: 119;
c) a tetravalent molecule comprising two copies of a polypeptide having the structure (GITR-BD)-Linker-(GITR-BD)-Linker-Hinge-Fc, wherein (i) the GITR-BD comprises a CDR1 comprising the sequence of SEQ ID NO: 120, a CDR2 comprising the sequence of SEQ ID NO: 121, and a CDR3 comprising the sequence of SEQ ID NO: 122, (ii) the Linker is a polypeptide, (iii) the Hinge is a polypeptide derived from an immunoglobulin hinge region, and (iv) the Fc is an immunoglobulin Fc polypeptide;
d) a tetravalent molecule comprising two copies of a polypeptide having the structure (GITR-BD)-Linker-(GITR-BD)-Linker-Hinge-Fc, wherein (i) the GITR-BD comprises the amino acid sequence of SEQ ID NO:119, (ii) the Linker is a polypeptide, (iii) the Hinge is a polypeptide derived from an immunoglobulin hinge region, and (iv) the Fc is an immunoglobulin Fc polypeptide; and
e) a tetravalent molecule comprising two copies of a polypeptide comprising the sequence of SEQ ID NO: 118.

68. The composition of claim 67, wherein the anti-GITR antibody is a tetravalent molecule having the structure (GITR-BD)-Linker-(GITR-BD)-Linker-Hinge-Fc, wherein (i) the GITR-BD comprises (a) a CDR1 comprising the sequence of SEQ ID NO: 120, a CDR2 comprising the sequence of SEQ ID NO: 121, and a CDR3 comprising the sequence of SEQ ID NO: 122 or (b) the sequence of SEQ ID NO:119, (ii) the Linker is a polypeptide comprising a sequence selected from SEQ ID NOs: 134-140, (iii) the Hinge is a polypeptide comprising a sequence selected from SEQ ID NOs: 129-133, and (iv) the Fc is an immunoglobulin Fc polypeptide comprising a sequence selected from SEQ ID NOs: 123-128.

69. Use of a composition comprising an anti-GITR antibody and an anti-CSF1R antibody for preparation of a medicament for treating pancreatic cancer in a subject according to the steps and/or conditions of any one of claims 40-68.

70. The use of claim 69, wherein the anti-GITR antibody is selected from:
a) an antibody comprising a GITR binding domain (GITR-BD) comprising a CDR1 comprising the sequence of SEQ ID NO: 120, a CDR2 comprising the sequence of SEQ ID NO: 121, and a CDR3 comprising the sequence of SEQ ID NO: 122;
b) an antibody comprising a GITR-BD comprising the sequence of SEQ ID NO: 119;
c) a tetravalent molecule comprising two copies of a polypeptide having the structure (GITR-BD)-Linker-(GITR-BD)-Linker-Hinge-Fc, wherein (i) the GITR-BD comprises a CDR1 comprising the sequence of SEQ ID NO: 120, a CDR2 comprising the sequence of SEQ ID NO: 121, and a CDR3 comprising the sequence of SEQ ID NO: 122, (ii) the Linker is a polypeptide, (iii) the Hinge is a polypeptide derived from an immunoglobulin hinge region, and (iv) the Fc is an immunoglobulin Fc polypeptide;
d) a tetravalent molecule comprising two copies of a polypeptide having the structure (GITR-BD)-Linker-(GITR-BD)-Linker-Hinge-Fc, wherein (i) the GITR-BD comprises the amino acid sequence of SEQ ID NO:119, (ii) the Linker is a polypeptide, (iii) the Hinge is a polypeptide derived from an immunoglobulin hinge region, and (iv) the Fc is an immunoglobulin Fc polypeptide; and
e) a tetravalent molecule comprising two copies of a polypeptide comprising the sequence of SEQ ID NO: 118.

71. The use of claim 70, wherein the anti-GITR antibody is a tetravalent molecule having the structure (GITR-BD)-Linker-(GITR-BD)-Linker-Hinge-Fc, wherein (i) the GITR-BD comprises (a) a CDR1 comprising the sequence of SEQ ID NO: 120, a CDR2 comprising the sequence of SEQ ID NO: 121, and a CDR3 comprising the sequence of SEQ ID NO: 122 or (b) the sequence of SEQ ID NO:119, (ii) the Linker is a polypeptide comprising a sequence selected from SEQ ID NOs: 134-140, (iii) the Hinge is a polypeptide comprising a sequence selected from SEQ ID NOs: 129-133, and (iv) the Fc is an immunoglobulin Fc polypeptide comprising a sequence selected from SEQ ID NOs: 123-128.

72. The use of any one of claims 69-71, wherein the anti-CSF1R antibody is selected from:
a) an antibody comprising a heavy chain comprising the sequence of SEQ ID NO: 39 and a light chain comprising the sequence of SEQ ID NO: 46;
b) an antibody comprising a heavy chain comprising a heavy chain (HC) complementarity determining region 1 (CDR1) comprising the sequence of SEQ ID NO: 15, an HC CDR2 comprising the sequence of SEQ ID NO: 16, and an HC CDR3 comprising the sequence of SEQ ID NO: 17, and a light chain comprising a light chain (LC) CDR1 comprising the sequence of SEQ ID NO: 18, a LC CDR2 comprising the sequence of SEQ ID NO: 19, and a LC CDR3 comprising the sequence of SEQ ID NO: 20; and
c) an antibody comprising a heavy chain comprising the sequence of SEQ ID NO: 53 and a light chain comprising the sequence of SEQ ID NO: 60.
